SEQUENCE LISTING (21 sequences)

SEQ ID NO: 1 - protein, 418 aa, Homo sapiens
MRLSAGPDAGPSGNSSPWWPLATGAGNTSREAEALGEGNGPPRDVRNEELAKLEIAVLAVTFAVAVLGNSSVLLALHRTPRKTSRMHLFIRHLSLADLAVAFFQVLPQMCWDITYRFRGPDWLCRVVKHLQVFGMFASAYMLVVMTADRYIAVCHPLKTLQQPARRSRLMIAAAWVLSFVLSTPQYFVFSMIEVNNVTKARDCWATFIQPWGSRAYVTWMTGGIFVAPVVILGTCYGFICYNIWCNVRGKTASRQSKGAEQAGVAFQKGFLLAPCVSSVKSISRAKIRTVKMTFVIVTAYIVCWAPFFIIQMWSVWDPMSVWTESENPTITITALLGSLNSCCNPWIYMFFSGHLLQDCVQSFPCCQNMKEKFNKEDTDSMSRRQTFYSNNRSPTNSTGMWKDSPKSSKSIKFIPVST

SEQ ID NO: 2 - DNA, 6402 bp, Homo sapiens
gaattctctgagtcagtgactattacctaattgcttgaaggattttttccagacaggtggtctggaaaccttttacctattaccttccatccctgaaccatttcaatcttctgcctcctggatatcttggagaaaatgaaccaacacaacacagctttcagtttttagagcatttcccccatacagaacattgtcttacttgatcttcccgatgacctcaacaacaggaaaggcaggtcctttcatttccatttataagacgcacagacccaggattatctagccacaggaagcaggactccagatttcaagtccagcatctcaacgtgacaaccttggtaactctgcatgaacggactggatagtaaagtggaattattactgagaactgcaatgaataaaatcttttgcattttttgcctacgtttcacagagggtgatatttttctgaggcaattaaatttataccacggccacaatactgaaacgttctgaccaacaaagtcatgctcctgcatctacacagcagataactgcagaaacggcttcctttcttccttgtaaaattgcctgaaaacagctcccccttgctgtccgtcgaggcatatcttcaccaacgttaaaacagagctgagggagatcgcatttctgcctccctcccgccctgcagaggggctccagctgttcagagtaacggattactaggtaggtggttgtttcccctccttcccagggcctctttcctctctttgagattgcctctttcttactcctgagcacaggagccgggcgggttttctgtcccttgccctggacagcactgcctggatggccgctgtccggcagctgctctttgtccacccaaaaagatgtccccacgactcagtagtaaccagacggtccccacggaccactgcggccaaatttccgccatccccgctgtgggaatcaggcttttcccgcagaaaaccccaggaatctagagaaaactccttaagtccctagtctccatagagaaaaccaggagacactccccccaaaccccgctgtgaatacaggcacagcagccactggggcctgaaagtgatgagtgcgttcttcccgtcgcaaacatagggtaataaatagcatgcatcaaagacgttactaggaagagatagctctttaagtcacgaggggggagaaatgtttgccccgggaaaatttgcctggggaataaaatttgccagactgctgcacgggtgagctcggtgagaaggaagaaacccggactggaggaggtgaggtcgagagccaggttcaggtgcaggagctagatgcgtggacgccggtgcgtggactggaggtttccaggtaccgcgcttagcgtgcctgttgaagtcaaatgcatggttaaggaggctagcgaggaaggctagtgagggaagcttgtggaaacggctacgagcccagaaaaggcatgactcgtcagttgtccaagtttttggaagggaaaagcgggaaagcgccacgatcccacctactgtgaggaggaatctgcgagtctcccagctccaccccctccacagtgatgcagaggacaaacaccgacgtagggagaggaaaaaataaaactccagggagcggggagtaggcaaccagcagtcttccggcaatagggcgggagggagcgcgtcccaaggaaacaagcaccgcataaatacttgagttgggaacccagtgcttccggaagctcggagctcaccttcccgacctcgccgaagttgaaaaaaggcagagcagggagaggggccagctcaccctgctgagagctgctcagtgggcaggcgggacgctgctccgggagacgcccactggagggatcgcagagcccggcaagctgcgagcgcgccaaagaccctgcgcttcggacgaggagcccaagtcctccgagacggggagggagcgcgccgcgagggctggagctccgaagagggccgagtaggagctgcatggacagcatgcgtctctccgccggtcccgacgcggggccctcgggcaactccagcccatggtggcctctggccaccggcgctggcaacacaagccgggaggccgaagccctcggggagggcaacggcccaccgagggacgtgcgcaacgaggagctggccaaactggagatcgccgtgctggcggtgactttcgcggtggccgtgctgggcaacagcagcgtactgctggctctgcaccggacgccgcgcaagacgtcccgcatgcacctcttcatccgacacctcagcctggccgacctggccgtggcattcttccaggtgctgccgcaaatgtgctgggacatcacctaccgcttccgcggccccgactggctgtgccgcgtggtgaagcacctgcaggtgttcggcatgtttgcgtcggcctacatgctggtagtcatgacagccgaccgctacatcgcggtgtgccacccgctcaagactctgcaacagcccgcgcgccgctcgcgcctcatgatcgcggccgcctgggtgctgagcttcgtgctgagcacgccgcagtacttcgtcttctccatgatcgaggtgaacaatgtcaccaaggcccgcgactgctgggccaccttcatccagccctggggttctcgtgcctacgtgacctggatgacgggcggcatctttgtggcgcccgtggtcatcttgggtacctgctacggcttcatctgctacaacatctggtgcaacgtccgcgggaagacggcgtcgcgccagagcaagggtgcagagcaagcgggtgtggccttccaaaaggggttcctgctcgcaccctgtgtcagcagcgtgaagtccatttcccgggccaagatccgcacggtgaagatgacttttgtgatcgtgacggcttacatcgtctgctgggcgcctttcttcatcatccagatgtggtctgtctgggatcccatgtccgtctggaccggtacgtgccgggaaaatagaggaaagtgcagggataggagtgtgtgtgtgtgtgtgtgtgtgtgtgtgagagagagagagagagagagagagaaaaaaaaatgagaatctagcaattttcttcatagtatcttctagggcagtagtttttaaacttctatgtgtacataagagtcgcaccttggaggaacactgtttaaaaaaaaaaaaagcatattcctggattccccatgcagacattctaattaagaatgtctggaatggggctcaggactctgcatttgtagtgtgcaatgatcctaagaccacaattccagaaactgtgtcctagagtcagccaagcaactacaaagccgaagaatgacttttgctaaatcactagctactgtattaacaccatttcaggtctattggattcagggaaatatttttactgtcacaactgctttttgttggattgtgaaaagtattacaattaattttaaaaatgtgtagaaatgcctcaggggcaaggatagaaacagatatatatttctaaagaaagctggagaaaaattctttagaaggtgagtaattccaatttggcatttagctaataatgttcctttctcgttataatctatttttttctaacaatgattaatagttttttcttggtatttcaaagcagaattattgataaactaaattcattagactaattcattagtctttcaccactcatttgaccacacggctctacatctctccacccaactcttacagtagtttactggaaatgttctctgccacctactattgtgtttgattttaattccttcccaaactagaaaaactaacttcacagtgacttgcaaaaaaaattatttaattttgcatcttgaaaatattttcttctagtaaagacaaaaactcaaactaaataaattccagtgcttgtcggaactcaaaccaaataaattcaatggtacagttgttaccagaatgtttctggtaacaggaaataggtatctgggagtagctgttctccttttgctttttggaattggagtaggggaggtatgtaatatgctttggaagttatatttgcaaataaaacatttcagtatgaatttaacttaaatattcttactgactataatactagcgataatgaaaaatacaatataaacactttatttttggtttgctatttcttatcttgcttgatcttagaagcctcttcatattgtccatcaaataaagaaattcagtctaattattgctttagcagaatttacactcaagtaataaaaacttcaattgtgcatagatatgttggtaattttcattctttgtgaataccatcttacccatggctcctgatcacctttgatagcagcatcttagcactaagtatgattaaataataacctgtaattgttttctggcataacaagagtgagaagatccaagtttatatttaataatcaaggaaaagtcagtgtttattgattattcttatttttagaaaaggtatattatcagcactgtagctccactgtgaaaggttataatatttatgcagtttaccagtgctaattatcataaaatattttagaatcctgttggaatttcctaattctactgttcttcttaatataatttgtttgaaccacaaccacagatggttttccaaatttctaaccaaagaaaaacaactaaagcttatatcatccagggacttcttctgtatggttttcatattataagaatatttaaaactactaaacttgatccctaatgcaatattttttcctgagttattaggataaatacaatttggtatacatggttatttaaaattatcttaaaatttcattacaattgtagccattctgtaactgctgtgtcattagcacatgctagttcgagtatagaagatagagattttttaaatcaattacttaatagtcttaacctcgtaaaattcccactcaattctatttaaatattttgatagtgttttaaaaatacttgaattaattttaaggcatcttgcttacaaaaatattttatagtcaagcaattttcaaacactccccatttccctgattgataaacaaaatagttcattttctatgataatccagaagtttatgccttcttaattagttaatagaaaaatgagtttatccatggttcacttacattacatgatttccctttatatttttcatgcagaatcggaaaaccctaccatcaccatcactgcattactgggttccttgaatagctgctgtaatccctggatatacatgttttttagtggccatctccttcaagactgtgttcaaagcttcccatgctgccaaaacatgaaggaaaaattcaacaaagaagatactgacagtatgagcagaagacagactttttattctaacaatcgaagcccaacaaacagtacgggtatgtggaaggactcgcctaaatcttccaagtccatcaaattcattcctgtttcaacttgagccttgcattcatgcaacttgattcttgtgattgactttttggctcattagctgaattgagctagaaatcacaagaacaaatacactttattaatataaccataaatcaattcattgtgtatgagactgtgtttctagttgcattttcatattgctaccaaaaactagacattattttgtatggaatattaatggaaacatgctgtactaaaatatgcaggtctgattcccagaaatacaacagaagttatatttttaaaggaaaaatcataaccaccctagctttatattttgttgttagtttcttttattttcatttctaacataagtaagacttgattggtttaaaagtcacataaaatgcggcactatttctgaacaaagagagctcatcatcagtcttaatattcagagaaaacttcagagaaattatgttttcatccattaaaattaatttgtgcatcagaaaatgcagccttaaacagtgtccaggagatgggatggtacctcctaggagtacaagtgcctggggtgtaatgagctcctgctcattgtggccagtttagagttctattagaagctatcaatcaccttgcatttcaaaatggtaactttacaactggcagtggcctccttttggttcctcacatattattggtcaagaaaagcatgaaaactgagatgctgaaggtgagaggaaatgttgactggccaaaaatatcttttttcccccactgcaaggttgttttaaagtcagatttgtataaggaaagccaaattttattaaaagagtagaaaaggattgcttaaggtactctggactttctcttggacattgtaaacgtattttgatcagtattacaagggtatcctgtgctatgctggacattaacaagatcattatcttcatgtttggggaattc

SEQ ID NO: 3 - protein, 164 aa, Homo sapiens
MPDTMLPACFLGLLAFSSACYFQNCPRGGKRAMSDLELRQCLPCGPGGKGRCFGPSICCADELGCFVGTAEALRCQEENYLPSPCQSGQKACGSGGRCAAFGVCCNDESCVTEPECREGFHRRARASDRSNATQLDGPAGALLLRLVQLAGAPEPFEPAQPDAY

SEQ ID NO: 4 - DNA, 621 bp, Homo sapiens
acagagccaccaagcagtgctgcatacggggtccacctgtgtgcaccaggatgcctgacaccatgctgcccgcctgcttcctcggcctactggccttctcctccgcgtgctacttccagaactgcccgaggggcggcaagagggccatgtccgacctggagctgagacagtgcctcccctgcggccccgggggcaaaggccgctgcttcgggcccagcatctgctgcgcggacgagctgggctgcttcgtgggcacggctgaggcgctgcgctgccaggaggagaactacctgccgtcgccctgccagtccggccagaaggcgtgcgggagcgggggccgctgcgccgccttcggcgtttgctgcaacgacgagagctgcgtgaccgagcccgagtgccgcgagggctttcaccgccgcgcccgcgccagcgaccggagcaacgccacgcagctggacgggccggccggggccttgctgctgcggctggtgcagctggccggggcgcccgagcccttcgagcccgcccagcccgacgcctactgagccccgcgctcgccccaccggcgcgctcttcgcgcccgcccctgcagcacggacaataaacctccgccaatgcaaa

SEQ ID NO: 5 - DNA, 21 bp, Artificial Sequence
  Description of Artificial Sequence Synthetic primer
tccaccaggtgagaagagtga

SEQ ID NO: 6 - DNA, 20 bp, Artificial Sequence
  Description of Artificial Sequence Synthetic primer
gaggctccaaaggcacttga

SEQ ID NO: 7 - DNA, 24 bp, Artificial Sequence
  Description of Artificial Sequence Synthetic primer
gggcatctgctgcagcgacgagag

SEQ ID NO: 8 - DNA, 21 bp, Artificial Sequence
  Description of Artificial Sequence Synthetic primer
agcgcgggtgaggcggaaaaa

SEQ ID NO: 9 - DNA, 21 bp, Artificial Sequence
  Description of Artificial Sequence Synthetic primer
tcccgtgccaagatccgcaca

SEQ ID NO: 10 - DNA, 24 bp, Artificial Sequence
  Description of Artificial Sequence Synthetic primer
ggtggaagggttttcggaatcggt

SEQ ID NO: 11 - DNA, 20 bp, Artificial Sequence
  Description of Artificial Sequence Synthetic primer
tctgtgtgggacgagaatgc

SEQ ID NO: 12 - DNA, 19 bp, Artificial Sequence
  Description of Artificial Sequence Synthetic primer
gcggtgactcagggaacgt

SEQ ID NO: 13 - DNA, 22 bp, Artificial Sequence
  Description of Artificial Sequence Synthetic primer
atctcaccttccaccttctgcg

SEQ ID NO: 14 - DNA, 23 bp, Artificial Sequence
  Description of Artificial Sequence Synthetic primer
cccgataatctccatcagtttcc

SEQ ID NO: 15 - DNA, 18 bp, Artificial Sequence
  Description of Artificial Sequence Synthetic primer
agtgccaggacctcacca

SEQ ID NO: 16 - DNA, 19 bp, Artificial Sequence
  Description of Artificial Sequence Synthetic primer
cagcgagaggtcgagtttg

SEQ ID NO: 17 - DNA, 20 bp, Artificial Sequence
  Description of Artificial Sequence Synthetic primer
aaggaaggctggaaaagagc

SEQ ID NO: 18 - DNA, 19 bp, Artificial Sequence
  Description of Artificial Sequence Synthetic primer
aaatcgtgcgtgacatcaa

SEQ ID NO: 19 - DNA, 50 bp, Homo sapiens
gtgtgtgtgtgtgtgtgtgtgtgtgtgtgtgtgtgtgtgtgtgtgtgtgt

SEQ ID NO: 20 - DNA, 74 bp, Homo sapiens
ctctctctttctctctctctctctctgtgtgtgtgtgtgtgtgtgtgtgtgtgtgtgtgtgtgtgtgtgtgtgt

SEQ ID NO: 21 - protein, 8 aa, Artificial Sequence
  Description of Artificial Sequence Synthetic peptide
  N-term [1-Mercaptocyclohexyl]acetyl
  MOD_RES (1)..(1): Tyr[O-Methyl]
  C-term NH2
YFQNCPRG

(19) United States
(12) Patent Application Publication
(10) Pub. No.: US 2018/0085423 A1
(43) Pub. Date: Mar. 29, 2018

(54) METHODS FOR TREATING AND DIAGNOSING EATING DISORDERS

(71) Applicant: THE TRUSTEES OF COLUMBIA UNIVERSITY IN THE CITY OF NEW YORK, New York, NY (US)

(72) Inventors: Lori Michelle ZELTSER, New York, NY (US); Rim HASSOUNA, New York, NY (US); Moneek MADRA, New York, NY (US)

(73) Assignee: THE TRUSTEES OF COLUMBIA UNIVERSITY IN THE CITY OF NEW YORK, New York, NY (US)

(21) Appl. No.: 15/784,927

(22) Filed: Oct. 16, 2017

Related U.S. Application Data
(63) Continuation-in-part of application No. PCT/US16/25209, filed on Mar. 31, 2016.
(60) Provisional application No. 62/148,698, filed on Apr. 16, 2015.

(51) Int. Cl. A61K 38/08; A61K 9/00

(52) U.S. Cl. CPC A61K 38/08; A61K 9/0019

(57) ABSTRACT

The present invention provides methods of treating or preventing an eating disorder, including anorexia nervosa, by administering a vasopressin V₁ₐ receptor (AVPR1A) antagonist. The present invention also provides methods of diagnosing the presence of or predisposition to an eating disorder, including anorexia nervosa, in a subject, comprising detecting the presence and/or level of arginine vasopressin (AVP) or AVPR1A in a the subject.

### RELATED APPLICATIONS

This application is a continuation-in-part of International Application No. PCT/US2016/025209, filed Mar. 31, 2016, which claims the benefit of priority to U.S. Provisional Patent Application No. 62/148,698, filed Apr. 16, 2015. The contents of these applications are incorporated herein by reference in their entirety.

### SEQUENCE LISTING

The instant application contains a Sequence Listing which has been submitted electronically in ASCII format and is hereby incorporated by reference in its entirety. Said ASCII copy, created on Oct. 16, 2017, is named 123756-00202_SL.txt and is 18,622 bytes in size.

### BACKGROUND

Anorexia nervosa is an eating disorder characterized by food restriction, odd eating habits or rituals, obsession with having a thin figure, and an irrational fear of weight gain. Anorexia nervosa is often coupled with a distorted self-image, which may be maintained by various cognitive biases that alter how individuals evaluate and think about their bodies, food, and eating. Individuals with anorexia nervosa often view themselves as overweight or not thin enough even when they are severely underweight. While the majority of individuals with anorexia nervosa continue to feel hunger, they deny themselves all but very small quantities of food.

Anorexia nervosa has the highest mortality rate of any psychiatric illness. The current standard of care, based on anorexia nervosa's high comorbidity with anxiety and depression, is to treat patients with psychotropic medications. Unfortunately, these pharmacological treatments have no impact on the restrictive eating behavior that is responsible for the high mortality rate of anorexia nervosa. Accordingly, there remains a need in the art for methods of treating anorexia nervosa that modulate the restrictive eating behaviors that drive anorexia nervosa's severe weight loss and high mortality rate.

### SUMMARY

The present invention is based, at least in part, on the discovery that vasopressin V₁ₐ receptor (AVPR1A) antagonists can modulate eating behavior in a subject. Thus, vasopressin V₁ₐ receptor antagonists may be used to treat or prevent eating disorders, such as anorexia nervosa. Accordingly, in one aspect, the present invention provides a method of modulating eating behavior in a subject to treat or prevent an eating disorder, e.g., anorexia nervosa, comprising the step of administering a vasopressin V₁ₐ receptor antagonist to the subject, to thereby treat or prevent the eating disorder in the subject. In one embodiment of the foregoing aspect, treating or preventing the eating disorder comprises increasing the feeding behavior of the subject.

In another aspect, the present invention provides a method of modulating eating behavior in a subject, e.g., increasing eating by the subject, increasing caloric intake by the subject, or decreasing caloric restriction by the subject, comprising the step of administering a vasopressin V₁ₐ receptor antagonist to the subject, to thereby modulate eating behavior in the subject.

In one embodiment of the foregoing aspects, the vasopressin V₁ₐ receptor antagonist can be, for example, a small molecule antagonist, such as, but not limited to, 1-(beta-mercapto-beta beta-cyclopentamethylenepropionic acid)-2-(O-methyl-Tyr)-argipressin, SR49049 (Relcovaptan), atosiban (Tractocile®), conivaptan (YM-087), VPA-985 (Lixivaptan), CL-385004, Vasotocin, SRX251 and SRX246 (Azevan), YM-218 (Astellas), OPC-2158 (Otsuka), and OPC21268, or a pharmaceutically acceptable salt thereof.

In another embodiment of the foregoing aspects, the vasopressin V₁ₐ receptor antagonist is selected from the group consisting of a small interfering RNA (siRNA), a microRNA, an antisense nucleic acid, a ribozyme, an antibody, and a peptide or peptide analogue.

In another embodiment of the foregoing aspects, wherein the vasopressin V₁ₐ receptor antagonist does not cross the blood brain barrier. In another embodiment of the foregoing aspects, the vasopressin V₁ₐ receptor antagonist does cross the blood brain barrier. In another embodiment of the foregoing aspects, the V₁ₐ receptor antagonist is administered via injection. In another embodiment of the foregoing aspects, the vasopressin V₁ₐ receptor antagonist is administered at a dose of about 1 μg/kg to about 50 μg/kg. In another embodiment of the foregoing aspects, the dose of the vasopressin V₁ₐ receptor antagonist is about 7 μg/kg. In another embodiment of the foregoing aspects, the dose of the vasopressin V₁ₐ receptor antagonist is about 40 μg/kg. In another embodiment of the foregoing aspects, the vasopressin V₁ₐ receptor antagonist is formulated as a pharmaceutical composition and comprises a pharmaceutically acceptable carrier.

In another embodiment of the foregoing aspects, the subject is a BDNF Val66Met variant carrier. In another embodiment of the foregoing aspects, the subject has a RS3 microsatellite polymorphism in the AVPR1A promoter region. In yet another embodiment of the foregoing aspects, the subject has experienced intentional or unintentional weight loss, e.g., due a calorie-restricting diet. In still another embodiment of the foregoing aspects, the subject is female. In yet another embodiment of the foregoing aspects, the subject is an adolescent. In another embodiment of the foregoing aspects, the subject is an adult that experienced social stressors during adolescence. In one embodiment of the foregoing aspects, the subject has, or is at risk of having, anxiety or an anxiety disorder. In a further embodiment of the foregoing aspects, the subject has a family history of an eating disorder.

In another aspect, the invention provides a method of determining whether a subject has or is at risk for developing an eating disorder, e.g., anorexia nervosa, the method comprising the steps of determining the level of arginine-vasopressin (AVP) in a biological sample obtained from the subject relative to the level of expression in a control sample, wherein increased expression of AVP indicates that the subject has or is at risk for an eating disorder. In one embodiment of the foregoing aspects, the method further comprising treating the subject with a vasopressin V₁ₐ receptor antagonist.

In another embodiment of the foregoing aspects, the subject is a BDNF Val66Met variant carrier. In another embodiment of the foregoing aspects, the subject has a RS3 microsatellite polymorphism in the AVPR1A promoter region. In yet another embodiment of the foregoing aspects, the subject has experienced intentional or unintentional weight loss, e.g., due to a calorie-restricting diet. In still another embodiment of the foregoing aspects, the subject is female. In yet another embodiment of the foregoing aspects, the subject is an adolescent. In another embodiment of the foregoing aspects, the subject is an adult that experienced social stressors during adolescence. In one embodiment of the foregoing aspects, the subject has, or is at risk of having, anxiety or an anxiety disorder. In a further embodiment of the foregoing aspects, the subject has a family history of an eating disorder.

In another aspect, the invention provides a method of determining whether a subject is a good candidate for treatment with a vasopressin V₁ₐ receptor antagonist, the method comprising the steps of determining the levels of arginine-vasopressin (AVP) in a biological sample obtained from the subject relative to the level of expression in a control sample, wherein increased expression of AVP indicates that the subject is a good candidate for treatment with a vasopressin V₁ₐ receptor antagonist.

In another aspect, the present invention also provides a method of determining whether a subject is a good candidate for treatment with a vasopressin V₁ₐ receptor antagonist, the method comprising the steps of determining the levels of arginine-vasopressin (AVP) in a biological sample obtained from the subject after administration of a hypertonic solution of saline relative to the level of expression in a control sample, wherein increased expression of AVP indicates that the subject is a good candidate for treatment with a vasopressin V₁ₐ receptor antagonist.

In yet another aspect, the invention provides a method for assessing the efficacy of a therapeutic agent for the treatment of an eating disorder, e.g., anorexia nervosa, in a subject, comprising the steps of: (a) determining the levels of arginine-vasopressin (AVP) in a biological sample obtained from the subject, prior to therapy with the therapeutic agent; and (b) determining the levels of AVP in a biological sample obtained from the subject, at one or more time points during therapy with the therapeutic agent, wherein the therapy with the therapeutic agent is efficacious for treating the eating disorder in the subject when there is a lower level of AVP in the second or subsequent samples, relative to the first sample. In one embodiment, the levels of AVP are determined after administration of a hypertonic solution of saline.

In one embodiment of the foregoing aspects, the biological sample is a blood sample, e.g., a serum sample.

In still another aspect, the present invention also provides a method of determining whether a subject has or is at risk for developing an eating disorder, or is a good candidate for treatment with a vasopressin V₁ₐ receptor antagonist, the method comprising the steps of determining the levels of AVPR1A in the amygdala of a subject relative to the level of expression in a control sample, wherein increased expression of AVPR1A in the amygdala of the subject indicates that the subject has or is at risk for developing an eating disorder, and/or is a good candidate for treatment with a vasopressin V₁ₐ receptor antagonist.

In one embodiment of the foregoing aspects, expression of AVPR1A is detected using a PET ligand to detect. In another embodiment of the foregoing aspects, the PET ligand is a radiolabeled vasopressin V₁ₐ receptor antagonist. In one embodiment of the foregoing aspects, the method further comprising treating the subject with a vasopressin V₁ₐ receptor antagonist.

In another embodiment of the foregoing aspects, the subject is a BDNF Val66Met variant carrier. In another embodiment of the foregoing aspects, the subject has a RS3 microsatellite polymorphism in the AVPR1A promoter region. In yet another embodiment of the foregoing aspects, the subject has experienced intentional or unintentional weight loss, e.g., due to a calorie-restricting diet. In still another embodiment of the foregoing aspects, the subject is female. In yet another embodiment of the foregoing aspects, the subject is an adolescent. In another embodiment of the foregoing aspects, the subject is an adult that experienced social stressors during adolescence. In one embodiment of the foregoing aspects, the subject has, or is at risk of having, anxiety or an anxiety disorder. In a further embodiment of the foregoing aspects, the subject has a family history of an eating disorder.

### BRIEF DESCRIPTION OF THE DRAWINGS

FIGS. 1A-C. hBDNF^{Met/?} genotype interacts with adolescent social stress to promote abnormal feeding behavior. FIG. 1A is a graph showing different patterns of daily food intake in hBDNF^{Met/?} females exposed to social isolation stress (GE). 79.4% of animals maintained normal food intake throughout (“NONE”). Of those that exhibited an aphagic episode (AE), 85.7% exhibited only one AE (“SINGLE”), while 14.3% exhibited repeated AEs “MULTIPLE”. 28.6% of the AEs resulted in death (“FATAL”). Time 0 d starts at 7 weeks of age. FIG. 1B is a bar graph showing the number of aphagic episodes (AE) per animal per group from 7 to 9.5 weeks of age in group-housed hBDNF^{Val/Val} (C) and hBDNF^{Met/?} (G) females; singly-housed hBDNF^{Val/Val} (E) and hBDNF^{Met/?} (GE) females (C, 0 AE/animal, n=10, 2 cohorts; G, 0 AE/animal, n=13, 3 cohorts; E, 0.07+/−0.07 AE/animal, n=14, 3 cohorts; GE, 0.23+/−0.08 AE/animal n=34, 9 cohorts). *P<0.05, ANOVA. FIG. 1C is a plot showing body weight changes within respective groups, each line represents one animal. Time 0 d starts at 7 weeks of age. (control (C), n=10; G, n=13; E, n=14; GE, n=34). Error bars denote s.e.m.

FIGS. 2A-E. Peri-pubertal caloric restriction synergizes with genetic and environmental factors to promote abnormal feeding behavior. FIG. 2A is a bar graph showing the number of aphagic episodes (AE) per animal per group from 7 to 9.5 weeks of age in singly-housed hBDNF^{Val/Val} (E) and hBDNF^{Met/?} (GE) females; or calorically-restricted singly-housed hBDNF^{Val/Val} (ED) and hBDNF^{Met/?} (GED) females (E, 0.07±0.07 AE/animal, n=14, 4 cohorts; GE, 0.23±0.08 AE/animal, n=34, 9 cohorts; ED, 0.18±0.95 AE/animal, n=17, 6 cohorts; GED, 0.61±0.14 AE/animal, n=36, 6 cohorts). *P<0.05, ANOVA. FIG. 2B is a bar graph showing the duration of AEs in ED and GED groups (ED, 1 day, n=2; GED, 1.3±0.1 days, n=15). *P<0.05, Student's t. FIG. 2C is a bar graph showing the number of AE per animal per group triggered by caloric restriction in singly-housed hBDNF^{Met/?} females (GED) and males (GED-M) (GED, 0.61±0.14 AE/animal, n=36, 6 cohorts; GEDM, 0.43±0.13 AE/animal, n=21, 3 cohorts). P<0.05, Student's t. FIG. 2D is a plot showing body weight changes within respective groups, each line represents one animal. Time 0 d starts at 7 weeks. (ED, n=17; GED, n=36). FIG. 2E is a bar graph showing body weight loss in ED and GED groups during AEs (ED, 17.2±1.5 g, n=2; GED 22.5±6.3 g, n=7). P<0.05, Student's t. Error bars denote s.e.m.

FIGS. 3A-D. Impacts of caloric restriction and social isolation are conveyed in the peripubertal period. FIG. 3A is a bar graph showing the number of aphagic episodes (AE) per animal per group in singly-housed hBDNF^{Met/?} females (GE) from 7-9.5 weeks of age compared to those observed after 9.5 weeks (GE: 7-9.5, 0.23±0.08 AE/animal; >9.5, 0.09±0.09 AE/animal, n=34, 9 cohort). FIG. 3B is a bar graph showing the number of AEs per animal per group in singly-housed hBDNF^{Met/?} females under caloric restriction (CR) (GED) from 7-9.5 weeks of age compared to those observed after 9.5 weeks (GED: 7-9.5, 0.61±0.14 AE/animal; >9.5, 0.14±0.06 AE/animal, n=36, 6 cohorts). *P<0.05, Student's t. FIG. 3C is a bar graph showing the number of AEs per animal per group in GE, GED, singly-housed hBDNF^{Met/?} females exposed to CR during adulthood (GED^{A}), and singly-housed hBDNF^{Met/?} females fed 100% of their daily caloric intake twice daily at 7 weeks (GED^{100%}) (GE, 0.23±0.08 AE/animal, n=34, 9 cohorts; GED, 0.61±0.14 AE/animal, n=36, 6 cohorts; GED^{A}, 0.20±0.14 AE/animal, n=15, 5 cohorts; GED^{100%}, 0.13±0.09 AE/animal, n=15, 2 cohorts). FIG. 3D is a bar graph showing the number of AEs per animal per group in GED, ED, hBDNF^{Met/?} and hBDNF^{Val/Val} females that were first exposed to single housing and CR during adulthood (GE^{A}D^{A}, E^{A}D^{A}), and singly-housed hBDNF^{Met/?} females exposed to daily handling for a week prior to CR (GEHD) (GED, 0.61±0.14 AE/animal, n=36, 6 cohorts; GE^{A}D^{A}, 0 AE/animal, n=10, 2 cohorts; GEHD, 0 AE/animal, n=10, 2 cohorts; ED, 0.18±0.95 AE/animal, n=17, 6 cohorts; E^{A}D^{A}, 0 AE/animal, n=10, 2 cohorts). *P<0.05, ANOVA. Error bars denote s.e.m.

FIGS. 4A-C. FIG. 4A is a bar graph showing the serum concentration of vasopressin at 9.5 weeks in singly-housed hBDNF^{Met/?} females under CR that experienced an AE from 7-9.5 weeks (GED-AE), singly-housed hBDNF^{Met/?} females under CR that did not have an AE (GED-Ø) and singly-housed hBDNF^{Met/?} females under CR and exposed to daily handling from 6 weeks (GEDH) (GED-Ø, 2.31±0.25 ng/mL, n=12, 5 cohorts; GED-AE, 5.21±0.19 ng/mL, n=6, 5 cohorts; GEDH, 3.45±0.21 ng/mL, n=5, 1 cohort). FIG. 4B is a bar graph showing the vasopressin receptor 1a (Avpr1a) expression levels in the pituitary (PIT) in GED-Ø, GED-AE, and GEDH females. (GED-Ø, 2.17±1.14 AU, n=12, 5 cohorts; GED-AE, 4.38±1.67 AU, n=4, 4 cohorts; GEDH, 0.78±0.16 AU, n=5, 1 cohort). FIG. 4C is a bar graph showing the Avpr1b expression levels in the pituitary in GED-Ø, GED-AE, and GEDH females (GED-Ø, 2.30±0.45 AU, n=12, 5 cohorts; GED-AE, 2.40±0.81 AU, n=4, 4 cohorts; GEDH, 1.34&0.35 AU, n=5, 1 cohort). *P<0.05, ANOVA. Error bars denote s.e.m.

FIGS. 5A-C. FIG. 5A is a bar graph showing serum concentration of vasopressin at 7 weeks in group-housed hBDNF^{Val/Val} (C) and ABDNF^{Met/?} (G) females; and singly-housed hBDNF^{Val/Val} (E) and hBDNF^{Met/?} (GE) females (C, 2.81±0.52 ng/mL, n=6, 2 cohorts; G, 4.04±0.51 ng/mL, n=5, 2 cohorts; E, 4.00±0.57 ng/mL, n=6, 1 cohort; GE, 6.90±0.75 ng/mL, n=8, 1 cohort). FIG. 5B is a bar graph showing vasopressin receptor 1a (Avpr1a) expression in the pituitary (PIT) in C, G, E, and GE females (C, 1.03±0.13 AU, n=4, 2 cohorts; G, 0.38±0.72 AU, n=7, 1 cohort; E, 0.46±0.31 AU, n=6, 1 cohort; GE, 0.40±0.09 AU, n=7, 1 cohort). FIG. 5C is a bar graph showing Avpr1b expression in the pituitary in C, G, E, and GE females (C, 1.08±0.23 AU, n=4, 2 cohorts; G, 0.89±0.25 AU, n=7, 1 cohort; E, 0.75±0.30 AU, n=6, 1 cohort; GE, 1.50±0.44 AU, n=8, 1 cohort). *P<0.05, ANOVA. Error bars denote s.e.m.

FIGS. 6A-E. FIG. 6A is a bar graph showing vasopressin (Avp) expression levels in the amygdala (AMG) at 7 weeks in group-housed hBDNF^{Val/Val} (C) and hBDNF^{Met/?} (G) females; or singly-housed hBDNF^{Val/Val} (E) and hBDNF^{Met/?} females (GE) (C, 1.03±0.13 AU, n=4, 2 cohorts; G, 0.38±0.72 AU, n=7, 1 cohort; E, 0.46±0.31 AU, n=6, 1 cohort; GE, 0.40±0.09 AU, n=7, 1 cohort). FIG. 6B is a bar graph showing vasopressin receptor 1a (Avpr1a) expression in the amygdala in C, G, E and GE females (C, 3.73±2.05 AU, n=4, 2 cohorts; G, 6.09±2.84 AU, n=7, 1 cohort; E, 2.18±1.41 AU, n=6, 1 cohort; GE, 16.66±4.82 AU, n=8, 1 cohort). FIG. 6C is a bar graph showing Avpr1a expression in the prefrontal cortex (PFC) in C, G, E and GE females (C, 1.79±0.85 AU, n=4, 2 cohorts; G, 1.86±0.34 AU, n=7, 1 cohort; E, 1.61±1.38 AU, n=6, 1 cohort; GE, 1.48±31 AU, n=8, 1 cohort). FIG. 6D is a bar graph showing Avpr1a expression in the hippocampus (HPC) in C, G, E and GE females (C, 1.99±1.09 AU, n=4, 2 cohorts; G, 0.55±0.09 AU, n=7, 1 cohort; E, 0.81±0.45 AU, n=6, 1 cohort; GE, 0.29±0.08 AU, n=7, 1 cohort). FIG. 6E is a bar graph showing Avpr1b expression in the HPC in C, G, E and GE females (C, 1.82±1.23 AU, n=4, 2 cohorts; G, 1.88±0.69 AU, n=6, 1 cohort; E, 1.55±0.12 AU, n=6, 1 cohort; GE, 2.10±0.50 AU, n=8, 1 cohort). *P<0.05, ANOVA. Error bars denote s.e.m.

FIGS. 7A-G. FIG. 7A is a bar graph showing total activity at 6-7 weeks in group housed, hBDNF^{Val/Val} (C) and hBDNF^{Met/?} (G) females, or singly-housed hBDNF^{Val/Val} (E) and hBDNF^{Met/?} (GE) females (C, 123198.3±19943.4 counts, n=3; G, 126131.7±18237.1 counts n=7; E, 155612.7±12822.76 counts, n=3; GE, 114137.8±8068.6 counts, n=14, 3 distinct cohorts). FIG. 7B is a bar graph showing normalized central activity counts in C, G, E and GE females at 6-7 weeks after exposure to a novel environment (C, 0.58±0.28 counts, n=3; G, 0.39±0.31 counts, n=7; E, 0.59±0.02 counts, n=3; GE, 0.48±0.04 counts, n=14, 3 distinct cohorts). FIG. 7C is a bar graph showing expression of Corticosterone releasing hormone (Crh) in the rostral ⅓ of the hypothalamus (R. HYP) (which contains the PVH) at 7 weeks in C, G, and GE females (C, 2.0±1.0 AU, n=4, 1 cohort; G, 5.5±1.7 AU, n=7, 1 cohort; GE, 5.3±2.4 AU, n=8, 1 cohort). FIG. 7D is a bar graph showing Crh receptor 1 (Crhr1) expression in the R. HYP at 7 weeks in C, G, and GE females (C, 1.3±0.4 AU, n=4, 1 cohort; G, 1.9±0.5 AU, n=7, 1 cohort; GE, 2.4±0.7 AU, n=8, 1 cohort). FIG. 7E is a bar graph showing Crhr2 expression in the R. HYP at 7 weeks in C, G, and GE females (C, 1.0±0.1 AU, n=4, 1 cohort; G, 1.3±0.4 AU, n=7, 1 cohort; GE, 1.6±0.3 AU, n=8, 1 cohort). *P<0.05, ANOVA. FIG. 7F is a bar graph showing baseline corticosterone levels at 9.5 weeks in singly-housed hBDNF^{Met/?} females under CR that did not have an AE (GED-Ø) compared to singly-housed hBDNF^{Met/?} females under CR that had an AE (GED-AE) (GED-Ø, 5.9±1.6 ng/dL, n=6, 2 cohorts; GED-AE, 4.88±0.7 ng/dL, n=6, 2 cohorts). FIG. 7G is a bar graph showing the absolute increase in corticosterone levels (restraint baseline) at 9.5 weeks in GED-Ø and GED-AE females. (GED-Ø, 32.5±3.9 ng/dL, n=6, 2 cohorts; GED-AE, 31.7±7.2 ng/dL, n=6, 2 cohorts). *P<0.05, Student's t. Activity data were obtained from the TSE ActiMot system. Error bars denote s.e.m.

FIGS. 8A-F. FIG. 8A is a bar graph showing the expression, as measured by quantitative PCR, of Corticosterone releasing hormone (Crh) in the rostral ⅓ of the hypothalamus (HYP) (which contains the PVH) at 7 weeks in group-housed, hBDNF^{Val/Val} (C) and hBDNF^{Met/?} (G) females, and singly-housed hBDNF^{Met/?} (GE) females (C, 2.0±1.0 AU, n=4, 1 cohort; G, 5.5±1.7 AU, n=7, 1 cohort; GE, 5.3±2.4 AU, n=8, 1 cohort). P<0.05, ANOVA. FIG. 8B is a bar graph showing the expression, as measured by quantitative PCR, of Pro-opiomelanocortin (Pomc) in the pituitary (PIT) at 7 weeks in C, G, and GE females (C, 1.2±0.4 AU, n=4, 1 cohort; G, 0.17±0.02 AU, n=3, 1 cohort; GE, 0.20±0.06 AU, n=4, 2 cohorts). P<0.05, ANOVA. FIG. 8C is a bar graph showing the baseline and restraint corticosterone levels at 7 weeks in C, G, and GE females (baseline: C, 7.8±3.2 AU, n=6, 1 cohort; G, 6.7±1.0 AU, n=7, 2 cohorts; GE, 5.9±2.3 AU, n=6, 2 cohorts; restraint: C, 37.7±4.6 AU, n=6, 1 cohort; G, 45.6±4.2 AU, n=7, 2 cohorts; GE, 39.0±0.5 AU, n=6, 2 cohorts). *P<0.05, ANOVA. FIG. 8D is a bar graph showing the weight of the adrenal glands, normalized to body weight, at 9.5 weeks in C, G, and GE females (C, 0.16±0.05 mg/g, n=5, 1 cohort; G, 2.3±1.1 mg/g, n=7, 1 cohort; GE, 1.4±0.3 mg/g, n=8, 1 cohort). P<0.05, ANOVA. FIG. 8E is a bar graph showing the normalized central activity counts in C, G, E and GE females at 6-7 weeks after exposure to a novel environment (C, 0.58±0.28 counts, n=3; G, 0.39±0.31 counts, n=7; E, 0.59±0.02 counts, n=3; GE, 0.48±0.04 counts, n=14, 3 distinct cohorts). ANOVA, lowercase letters above bars denote similar (P>0.05) groups. FIG. 8F is a bar graph showing the total activity at 6-7 weeks in C, G and GE females (C, 123198.3±19943.4 counts, n=3; G, 126131.7±18237.1 counts n=7; E, 155612.7±12822.76 counts, n=3; GE, 114137.8±8068.6 counts, n=14, 3 distinct cohorts). P<0.05, ANOVA. Error bars denote s.e.m.

FIGS. 9A-B. FIG. 9A is a bar graph showing the number of AEs per animal per group in singly-housed hBDNF^{Met/?} females not exposed to daily handling (GED) and GED females exposed to daily handling for one week prior to CR (GEDH) (GED, 0.61±0.14 AE/animal, n=36, 6 cohorts; GEHD, 0 AE/animal, n=10, 2 cohorts). *P<0.05, Student's t. FIG. 9B is a bar graph showing the corticosterone levels at baseline (0 min) and after 15 minutes immobilization stress (restraint) in 9.5 week old singly-housed hBDNF^{Met/?} females exposed to caloric restriction (CR) (GED) and handling (GEDH) (baseline: GED, 6.75+/−1.59 ug/dL, n=13, 3 cohorts; GEDH, 4.65+/−0.58 ug/dL, n=12, 2 cohorts; restraint: GED, 38.20+/−3.88 ug/dL, n=13, 3 cohorts; GEHD, 53.51+/−4.03 ug/dL, n=12, 2 cohorts). *p<0.05, Student's t; baseline to restraint. #p<0.05, Student's t; GED to GEDH. Error bars denote s.e.m.

FIGS. 10A-D show that the G×E interactions at 7 wks increase serum AVP (FIG. 10A) and expression of Avp (FIG. 10B), Avpr1a (FIG. 10C) and P75Ntr/Ngfr (FIG. 10D) in the amygdala.

FIG. 11 shows the effect of i.p. injections of saline (circle, triangle) versus Compound 1 (square, diamond) on food intake in GED (circle, square) and wild-type (triangle, diamond).

FIGS. 12A-I show that hyperactivity of the neuroendocrine AVP system is associated with risk of anorexia-like behavior. 9.5 week-old GED females that exhibited an aphagic episode (GED-AE) were compared versus age- and exposure-matched littermates that did not (GED-Ø) (FIG. 12A). A history of aphagic behavior (GED-AE) was associated with a 3-fold increase in Avp in the rostral hypothalamus (which did not reach significance) and a 2.75-fold increase in Avp receptor 1A (Avpr1a) expression in the anterior pituitary (P<0.05, n=10-12) (FIG. 12B); Avpr1b and Oxytocin receptor (Oxtr) expression were not different (FIG. 12C (Avpr1b) and FIG. 12D (Oxtr)). Levels of serum AVP in response to avertin anesthesia were increased in GED-AE mice, but were the same in awake mice (FIG. 12E). Exposing hBDNFMet carriers to social isolation stress was associated with increased circulating AVP in response to anesthesia at 7 weeks (GE, stippled bars in FIG. 12I), baseline levels were not altered (GE, unfilled bars in FIG. 12I). Expression of hypothalamic Avp (data not shown) and pituitary Avpr1a (FIG. 12F), Avpr1b (FIG. 12G) and Oxtr (FIG. 12H) were not significantly different between the groups.

FIGS. 13A-C show that AVP-AVPR1A signals in the central amygdala are sufficient to promote anorexic behavior. FIG. 13A illustrates the location of AVP injection in the amygdala. FIG. 13B shows the anorexic effect of intra-amygdala AVP (0.03 ng) in wild-type mice, wherein food intake was reduced. The same anorexic effect is not observed in Avpr1a^{-/-} mutants (AVPR1a KO) (FIG. 13C). ANOVA, *P<0.05. Error bars denote s.e.m. Wild-type n=6 per group. AVPRAa^{-/-} mutant n=4 per group.

FIGS. 14A-B show that peripheral injection of an AVPR1A antagonist, Compound 1, can reverse anorexigenic behavior. FIG. 14A shows that peripheral injection of compound 1 is able to reverse hypophagia induced by central injection of 3 ng AVP vs. saline into fasted wild-type females while peripheral injections of saline, or SR49059 did not have that effect. FIG. 14B shows that GED mice injected with Compound 1 consumed significantly more food than saline-injected GED mice 1 hour and 2 hours after treatment, while injection with the SR49059 compound did not enhance food intake. Saline, avp+saline, avp_manning (Compound 1), avp+SR49059; n=5, 4, 5, 4). GED-AE saline, manning (Compound 1), SR49059; n=4, 4, 3. WT saline, manning (Compound 1), SR49059; n=10, 5, 5.

### DETAILED DESCRIPTION OF THE INVENTION

The present inventors have developed a mouse model of anorexia nervosa that combines both genetic and environmental factors that are consistently associated with increased risk of anorexia nervosa (AN) in humans—adolescent females, genetic predisposition to anxiety (“G”, imposed by the brain-derived neurotrophic factor (BDNF) gene variant hBDNF-Val66Met allele), social stress (“E”, environmental stress due to social isolation), and dieting (“D”, approximately 25% restriction of caloric intake)—to define a set of conditions that elicit anorexia nervosa-like behavior in these animals. As described in detail in Example 1, below, the inventors found that roughly half of the female Val66Met mice with all of these risk factors exhibit severe self-imposed dietary restriction, hereinafter referred to as an “aphasic episode” (AE), that is usually accompanied by significant body weight loss, sometimes to the point of death.

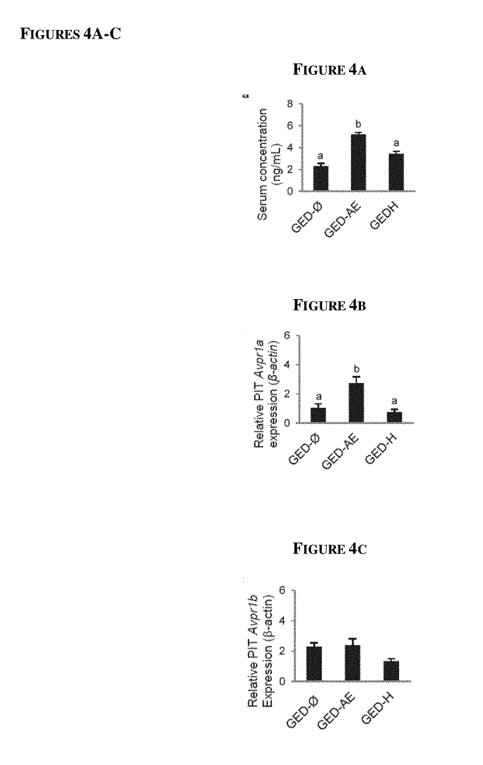

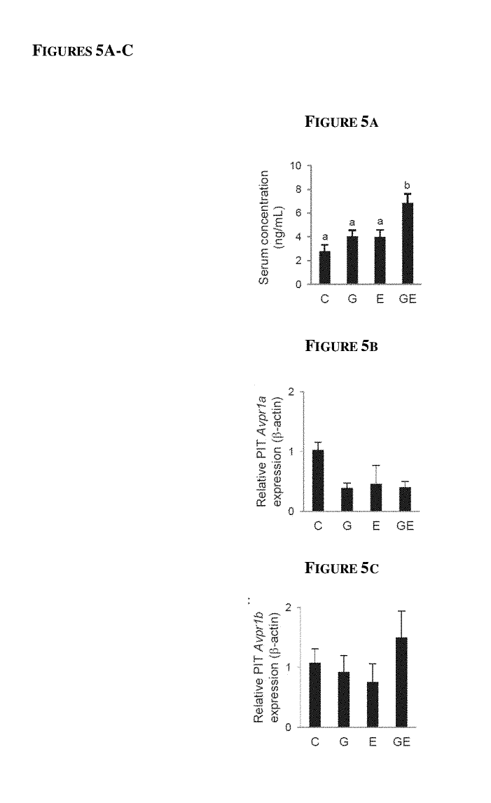

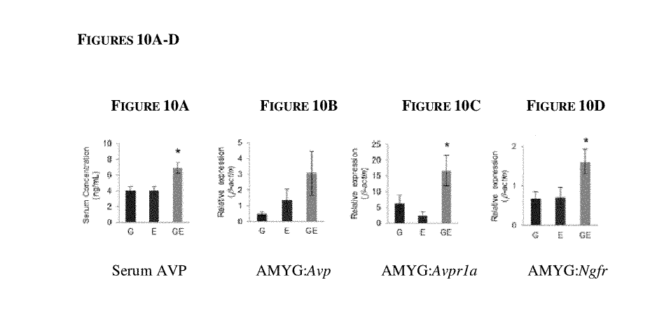

As described herein, this mouse model was used to identify “Gene×Environment×Diet” interactions that contribute to the incidence of abnormal feeding behavior, and is referred to herein as the GED model. By comparing age- and exposure-matched mice that exhibited anorexia-like behavior (GED-AE) from those that did not (GED-Ø), the neuroanatomical and molecular correlates of subsequent risk of anorexic behavior can be identified. As described in detail in Example 1, the inventors have discovered that levels of arginine vasopressin (AVP) in the serum were more than 2-fold higher in mice that exhibited anorexic behavior (FIG. 4A). Moreover, it was observed that exposure of Val66Met carriers to social isolation stress was associated with increased serum AVP levels at 7 weeks of age, even before the onset of anorexia nervosa-like behavior (FIG. 10A).

The inventors have also found that elevated expression of the AVP receptor vasopressin V₁ₐ receptor (Avpr1a) and the BDNF p75^{NTR} receptor (Ngfr) in the amygdala (and not in the hippocampus or prefrontal cortex) could be detected in singly-housed Val66Met carriers in mice 7 weeks old, even before the onset of anorexia nervosa-like behavior (FIG. 10C-D). In older mice (e.g., 9 weeks), Avpr1a expression in the anterior pituitary was higher in mice that had previously exhibited aphagic behavior, as compared to age- and exposure-matched mice that ate normally.

Without wishing to be bound by any particular theory, these data, in combination, indicate that adolescent social stress increases BDNF to p75^{NTR} signaling in the amygdala to promote AVP release, and in turn, that signaling through AVPR1A contributes to severe dietary restriction. Thus, based on these studies, the inventors have found that increased AVPR1A signaling contributes to anorexic behavior, including abnormal eating behavior. Moreover, as set forth in Example 3, the inventors have shown that administration of an AVPR1A antagonist to GED mice can increase food intake during an aphagic episode in these mice. The inventors have also found that AVP is a serum biomarker that can be used to predict sensitivity to caloric restriction resulting in an eating disorder, e.g., anorexia nervosa.

Accordingly, the present invention is based, at least in part, on the discovery that AVPR1A antagonists can be used to treat or prevent eating disorders, including anorexia nervosa. Thus, in certain non-limiting embodiments, the present invention provides methods for treating or preventing an eating disorder, e.g., anorexia nervosa, in a subject, by administering an AVPR1A antagonist to the subject. In one embodiment, administration of an AVPR1A antagonist modulates feeding behavior in the subject, e.g., increases eating by the subject, increases caloric intake by the subject, or decreases caloric restriction by the subject.

Furthermore, the present invention is based, also in part, on the discovery that AVP and AVPR1A can serve as a predictive biomarkers to diagnose or predict risk of a subject for developing an eating disorder, e.g., anorexia nervosa, or to identify whether a subject having an eating disorder, e.g., anorexia nervosa, is a good candidate for treatment with an AVPR1A antagonist. Thus, in other non-limiting embodiments, the present invention provides methods for diagnosing an eating disorder, e.g., anorexia nervosa, in a subject, or identifying a subject as a good candidate for treatment with an AVPR1A antagonist, by detecting elevated AVP in a biological sample, e.g., a blood or serum sample, obtained from the subject. In one embodiment, the levels of AVP in a biological sample obtained from the subject are determined after administration of a hypertonic solution of saline.

In anther non-limiting embodiment, the present invention provides methods for diagnosing an eating disorder, e.g., anorexia nervosa, in a subject, or identifying a subject as a good candidate for treatment with an AVPR1A antagonist, by detecting elevated AVPR1A in subject, e.g., in the amygdala of the subject.

### Definitions

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

As defined herein, the terms “modulate”, “modulating”, and “modulation” of feeding or eating behavior means adjusting, e.g., increasing, the amount of food intake and/or the rate of feeding of a subject.

As defined herein, the terms “treat”, “treatment”, and “treating” are to be understood accordingly as embracing prophylaxis and treatment or amelioration of symptoms of disease as well as treatment of the cause of the disease, including an eating disorder such as anorexia nervosa. For purposes of this subject matter, beneficial or desired clinical results include, but are not limited to, alleviation or amelioration of one or more symptoms, diminishment of extent of disease, stabilized (i.e., not worsening) state of disease, prevention of disease, delay or slowing of disease progression, and/or amelioration or palliation of the disease state. “Treatment” can also mean decreasing the severity or preventing a particular eating disorder-related symptom, e.g., regurgitating food after eating; refusing to eat; caloric restriction, avoiding certain foods; restricting and/or limiting food or caloric intake; eating only certain types of food; “picking” at food; expressing depression, disgust, shame or guilt about eating habits; skipping meals or making excuses for not eating; excessively focusing on healthy eating; adopting an overly restrictive vegetarian diet; making own meals rather than eating what the family eats, excessive overeating, etc. or physical symptoms caused by the aforementioned, such as bone loss, absent or irregular menstruation, loss of skin integrity, and heart complications, as described below. The decrease can be a 2%, 5%, 10%, 20%, 30%, 40%, 50%, 60%, 70%, 80%, 90%, 95%, 98% or 99% decrease in severity of complications or symptoms. “Treatment” can also mean prolonging survival as compared to expected survival if not receiving treatment.

As used herein, the term “subject” includes a human (male or female) subject, although it is to be understood that the methods described herein are effective with respect to all vertebrate species, which are intended to be included in the term “subject”. Accordingly, a “subject” can include a human subject for medical purposes, such as for the treatment of an existing condition or disease or the prevention of the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, a fetal, neonatal, infant, juvenile, or adult subject. The terms “subject” and “patient” are used interchangeably herein.

The term “eating disorder”, as used herein, refers to a group of illnesses defined by abnormal eating habits that may involve either insufficient or excessive food intake to the detriment of an individual's physical and/or mental health. Various eating disorders are specified as mental disorders in standard medical manuals, such as in the ICD-10, the DSM-5, or both, including, for example, anorexia nervosa, bulimia nervosa, binge-eating disorder (BED), and otherwise-specified feeding or eating disorders. Other eating disorders have been described by the mental health community, but are not yet recognized in standard medical manuals, including, for example, compulsive overeating, diabulimia, orthorexia nervosa, selective eating disorder, drunkorexia, and pregorexia.

Each of the aforementioned eating disorders are characterized by a set of symptoms and behaviors, which can overlap with other eating disorders. For example, anorexia nervosa is characterized by lack of maintenance of a healthy body weight, an obsessive fear of gaining weight or refusal to do so, and an unrealistic perception, or non-recognition of the seriousness, of current low body weight. Clinical presentation of anorexia nervosa often originates with self-imposed malnutrition resulting in a BMI of less than 17.5 or falling below the 10th BMI for age percentile (Herpertz, S., et al., German Society of Psychosomatic, M., Psychotherapy, and German College for Psychosomatic, M. (2011) The diagnosis and treatment of eating disorders, Deutsches Arzteblatt international 108, 678-685). Definitive diagnosis of disease can be based on DSM-V criteria following psychiatric exam (Association, A. P. (2013) Diagnostic and statistical manual of mental disorders (5th ed.)).

Beyond the overt weight loss and food restriction, anorexia nervosa can result in an array of associated pathologies contributing to the severity of disease and in some instances death. These include complications in the cardiovascular, gastrointestinal, electrolyte, endocrine, renal and hematologic systems. Anorexia nervosa can cause menstruation to stop (amenorrhoea), and often leads to bone loss (e.g., osteoporosis), loss of skin integrity, etc; anorexia nervosa also greatly stresses the heart, increasing the risk of heart attacks and related heart problems; and the risk of death is greatly increased in individuals with this disease. Other effects of anorexia nervosa include, but are not limited to dry skin, dry or chapped lips, poor circulation, headaches, bruising easily, decreased libido, impotence in males, reduced metabolism, abnormally slow heart rate, low blood pressure, hypotension, hypothermia, anaemia, abdominal pain, oedema, stunting of height and growth, abnormal mineral and electrolyte levels, thinning of the hair, lanugo, zinc deficiency, reduction in white blood cell count, reduced immune system function, constipation or diarrhoea, tooth decay, etc. In addition, depression, anxiety, obsessive compulsive disorder (OCD), post-traumatic stress disorder, personality disorders and substance abuse disorders are examples of psychiatric illnesses that are co-morbid with anorexia nervosa.

Eating disorders also include, but are not limited to, otherwise-specified feeding or eating disorders, which is an eating or feeding disorder that does not meet full DSM-5 criteria for anorexia nervosa, bulimia nervosa, or binge-eating disorder. Examples of otherwise-specified eating disorders include individuals with atypical anorexia nervosa, who meet all criteria for anorexia nervosa except being underweight, despite substantial weight loss; atypical bulimia nervosa, who meet all criteria for bulimia nervosa except that bulimic behaviors are less frequent or have not been ongoing for long enough; purging disorder; and night eating syndrome. Selective eating disorder, also called picky eating, is an extreme sensitivity to how something tastes. Drunkorexia is commonly characterized by purposely restricting food intake in order to reserve food calories for alcoholic calories, exercising excessively in order to burn calories consumed from drinking, and over-drinking alcohols in order to purge previously consumed food. Pregorexia is characterized by extreme dieting and over-exercising in order to control pregnancy weight gain. In a particular embodiment, the subject has, or is at risk of having, anorexia nervosa. In another embodiment, the subject has, or is at risk of having, an eating disorder that is closely-related to anorexia nervosa, including, for example, an otherwise-specified eating disorder, selective eating disorder, drunkorexia, and/or pregorexia.

As used herein, the term “vasopressin V₁ₐ receptor” or “AVPR1A” refers to one of three major receptor types for arginine vasopressin (AVPR1B, AVPR2, and Oxytocin receptor (OXTR) being the others), and is present throughout the brain, as well as in the periphery in the liver, kidney, and vasculature. AVPR1A is a G-protein coupled receptor (GPCR) with 7 transmembrane domains that couple to Gaq/11 GTP binding proteins, which along with Gb1, activate phospholipase C activity. Human AVPR1A cDNA is 1472 bp long and encodes a 418 amino-acid long polypeptide. Exemplary, non-limiting GENBANK Accession Numbers for AVPR1A human protein and DNA are AAB19232 (SEQ ID NO: 1) and U19906 (SEQ ID NO: 2), respectively.

An “AVPR1A biological activity” includes, but is not limited to, binding to arginine vasopressin (AVP) and promotion of abnormal eating behavior including dietary restriction.

As used herein, the term “vasopressin V₁ₐ receptor antagonist” or “AVPR1A antagonist” refers to an agent (e.g., a molecule) that inhibits or blocks one or more AVPR1A biological activity, including, e.g., including, but not limited to, binding AVP and promotion of abnormal eating behavior including dietary restriction. Preferably, the AVPR1A antagonists according to the invention act through direct interaction with the AVPR1A receptor. Antagonists include, but are not limited to, a molecule that blocks, inhibits, or reduces the expression or biological activity of a vasopressin V₁ₐ receptor gene product. AVPR1A antagonists can include small interfering RNA (siRNA), microRNA, antisense nucleic acid, a ribozyme, an antibody, a peptide, and a small molecule, or any other molecules which bind or interact with the vasopressin V₁ₐ receptor gene product. In one embodiment, an AVPR1A antagonist can be an antagonist that functions to antagonize AVPR1A in the pituitary or in the amygdala. In another embodiment, the AVPR1A antagonist is capable of crossing the blood brain barrier.

As used herein, the term “PET imaging” refers to positron emission tomography, which is a nuclear medicine, functional imaging technique that produces a three-dimensional image of functional processes in the body. A “PET scanner” is an imaging device that can detect a radioactive substance, called a tracer or PET ligand, in order to look for disease in the body. A PET scan shows how organs and tissues are working. The system detects pairs of gamma rays emitted indirectly by a positron-emitting radionuclide (tracer), which is introduced into the body on a biologically active molecule. Three-dimensional images of tracer concentration within the body are then constructed by computer analysis.

The term “antibody,” as used herein, refers to an intact antibody, or a binding fragment thereof that competes with the intact antibody for specific binding and includes chimeric, humanized, fully human, and bispecific antibodies. In certain embodiments, binding fragments are produced by recombinant DNA techniques. In additional embodiments, binding fragments are produced by enzymatic or chemical cleavage of intact antibodies. Binding fragments include, but are not limited to, Fab, Fab′, F(ab′)2, Fv, immunologically functional immunoglobulin fragments, heavy chain, light chain, and single-chain antibodies.

In general, the “effective amount” of a vasopressin V₁ₐ receptor antagonist refers to the amount necessary to elicit the desired biological response. As will be appreciated by the skilled artisan, the effective amount of a vasopressin V₁ₐ receptor antagonist may vary depending on such factors as the desired biological endpoint, the agent to be delivered, the composition of the encapsulating matrix, the target tissue, and the like.

As used herein, the term “arginine vasopressin” or “(AVP)”, refers to a neurohypophysial hormone. It is derived from a preprohormone precursor that is synthesized in the hypothalamus and stored in vesicles at the posterior pituitary. The majority is stored in the posterior pituitary to be released into the bloodstream. However, some AVP may also be released directly into the brain, and may play a role in social behavior, sexual motivation and pair bonding, and maternal responses to stress. Exemplary, non-limiting GENBANK Accession Numbers for AVP human protein and mRNA sequences are NP_000481 (SEQ ID NO:3) and NM_000490 (SEQ ID NO:4), respectively.

As used herein, the term “biomarker” refers to a marker (e.g., an expressed gene, including mRNA and/or protein) that allows detection of a disease in an individual, including detection of disease in its early stages (e.g., prior to onset of symptoms), or prediction of whether a subject is a good candidate for treatment with a particular therapy, e.g., a vasopressin V₁ₐ receptor antagonist. In one embodiment, a biomarker, as used herein, is an AVP nucleic acid and/or protein. In another embodiment, a biomarker, as used herein, is an AVPR1A nucleic acid and/or protein.

In specific embodiments, the expression level of a biomarker as determined by protein or nucleic acid levels in biological sample from an individual to be tested is compared with respective levels in normal biological sample from a control, e.g., a healthy individual. In certain non-limiting embodiments, a biomarker is a released and/or secreted protein that can be detected in a biological sample of a subject.

As used herein, the term “biological sample” refers to a sample of biological material obtained from a subject, preferably a human subject, including a biological fluid, e.g., blood (including serum or plasma).

Following long-standing patent law convention, the terms “a”, “an”, and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise”, “comprises”, and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off; measurement error and the like, and other factors known to those of skill in the art depending on the desired properties sought to be obtained by the presently disclosed subject matter. For example, the term “about”, when referring to a value can be meant to encompass variations of; in some embodiments±100%, in some embodiments±50%, in some embodiments±20%, in some embodiments±10%, in some embodiments±5%, in some embodiments±1%, in some embodiments±0.5%, and in some embodiments±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof subsumed within that range (for example, the recitation of 1 to 5 includes 1, 2, 3, 4, and 5, as well as fractions thereof e.g., 1.5, 2.25, 3.75, 4.1, and the like) and any range within that range.

### Therapeutic Methods

In one non-limiting aspect, the present invention provides a method of modulating (e.g., increasing) eating behavior, such as that related to an eating disorder, e.g., anorexia nervosa, in a subject comprising administering a vasopressin V₁ₐ receptor antagonist, a pharmaceutically acceptable salt thereof; or a pharmaceutical composition comprising a vasopressin V₁ₐ receptor antagonist, to the subject.

In another aspect, provided herein is a method of treating or preventing an eating disorder, e.g., anorexia nervosa, in a subject comprising administering to the subject a vasopressin V₁ₐ receptor antagonist, a pharmaceutically acceptable salt thereof or a pharmaceutical composition comprising a vasopressin V₁ₐ receptor antagonist, thereby treating or prevent an eating disorder, e.g., anorexia nervosa, in the subject.

In certain embodiments of the foregoing methods, the subject is a Brain-derived neurotrophic factor (BDNF)-Val66Met carrier. BDNF, is a protein that, in humans, is encoded by the BDNF gene. BDNF is a member of the neurotrophin family of growth factors.

Neurotrophic factors are found in the brain and the periphery. BDNF acts on certain neurons of the central nervous system and the peripheral nervous system, helping to support the survival of existing neurons, and encourage the growth and differentiation of new neurons and synapses. A common single-nucleotide polymorphism in the brain-derived neurotrophic factor (BDNF) gene is a methionine (Met) substitution for valine (Val) at codon 66 (Val66Met) (Chen, Z. Y., et al. Science 314, 140-143 (2006); Cao, L., et al. Curr Biol 17, 911-921 (2007)). The BDNF-Val66Met polymorphism has been associated with a wide range of psychiatric disorders, including anxiety-related affective and eating disorders (Gratacos, M., et al. Biol Psychiatry 61, 911-922 (2007)).

Val66Met is variant that is carried by 20-30% of Caucasian populations and up to 72% of certain Asian subpopulations (Petryshen, T. L., et al. (2010) Molecular Psychiatry 15, 810-815). The polymorphism is caused by a nucleotide substitution of adenine for guanine at position 196, resulting in the replacement of the 66^{th} amino acid valine (Val) with methionine (Met) within the prodomain (Phillips, C., et al. (2014) Frontiers in cellular neuroscience 8, 170). This substitution causes a structural change in the protein converting a β-sheet to an α-helical conformation (Anastasia, A., et al. (2013) Nature communications 4, 2490), resulting in decreasing binding to sortilin, a vacuolar protein sorting/targeting protein 10 (Vps10), which is involved in the activity-dependent release of BDNF (Ninan, I. (2014) Neuropharmacology 76 Pt C, 684-695).

In other embodiments of the foregoing methods, the subject has an RS3 microsatellite polymorphism in the AVPR1A promoter region. Variation in a repetitive microsatellite element in the 5′ flanking region of AVPR1A in rodents has been associated with variation in brain vasopressin 1a receptor expression and in social behavior. In humans, the 5′ flanking region of AVPR1A contains a tandem duplication of two ˜350 bp, microsatellite-containing elements located approximately 3.5 kb upstream of the transcription start site. The first block, referred to as DupA, contains a polymorphic (GT)₂₅ microsatellite (SEQ ID NO: 19); the second block, DupB, has a complex (CT)₄-(TT)-(CT)₈-(GT)₄ polymorphic motif (SEQ ID NO: 20), known as RS3. Relative to all other alleles, the 334 allele of RS3 shows overactivation of left amygdala (in response to fearful face stimuli), with longer variants of RS3 additionally associated with stronger amygdala activation. Polymorphisms in RS3 have been associated with variation in sociobehavioral traits in humans, including autism spectrum disorders, anxiety and depression, and eating disorders (see Bachner-Melman Int J Eat Disord 36: 451-460 (2004)).

In one embodiment, the subject is a patient aflicted with or suspected of being afflicted with a condition or disease, such as an eating disorder, e.g., anorexia nervosa. In still other embodiments, the subject is an individual having a history of past eating disorders. In another embodiment, the subject has a family history of eating disorders.

In some embodiments, the subject has experienced intentional or unintentional weight loss. In particular embodiments, the intentional or the unintentional weight loss is due to a calorie-restricting diet.

In one embodiment, the subject is an adolescent. In another embodiment, the subject is a female. In another embodiment, the subject is an adult that has experienced social stressors during adolescence or an adolescent that has experienced social stressors. Social stressors during adolescence include, but are not limited to, physical and/or emotional abuse, isolation, displacement, and the like.

In some embodiments, the subject has, or is at risk of having, anxiety or an anxiety disorder. Anxiety disorders are a category of mental disorders characterized by feelings of anxiety and fear, where anxiety is a worry about future events and fear is a reaction to current events. These feelings may cause physical symptoms, such as a racing heart and shakiness. There are a number of anxiety disorders, including, but not limited to, generalized anxiety disorder, a specific phobia, social anxiety disorder, separation anxiety disorder, agoraphobia, panic disorder, and others. While each has its own characteristics and symptoms, they all include symptoms of anxiety.

In some embodiments, a vasopressin V₁ₐ receptor antagonist can be used to treat or prevent an eating disorder, e.g., anorexia nervosa, in combination with one or more additional eating disorder therapeutics. For example, pharmacotherapy for anorexia nervosa can include antidepressant (selective serotonin reuptake inhibitors (SSRIs) and tricyclic antidepressants (TCAs)), atypical antipsychotic, and anti-anxiety medications. Though SSRIs are effective in treatment of depression and anxiety when not associated with anorexia nervosa, they have no effect on feeding endpoints. Other treatment strategies that can be used with a vasopressin V₁ₐ receptor antagonist include, but are not limited to, cognitive behavioral therapy (CBT), interpersonal psychotherapy, psychodynamic therapy, and family-based therapy.

Additional treatments for other symptoms and effects of anorexia nervosa may be also be administered in combination with a vasopressin V₁ₐ receptor antagonist, including treatment for heart rhythm disturbances, dehydration, electrolyte imbalances, other physical effects, or related psychiatric issues.

### AVPR1A Receptor Antagonist

Suitable vasopressin V₁ₐ receptor (AVPR1A) antagonists for use as described herein are compounds having antagonist activity at the vasopressin V₁ₐ receptor. Suitable vasopressin receptor antagonists for use in the present invention include compounds that suppress the vasopressin V₁ₐ receptor, but also may have antagonist activity at one or more other related receptors, such as the V_{1b}, V₂, or OT receptors. In one embodiment, a vasopressin V₁ₐ receptor antagonist for use according to the present disclosure has greater activity at the vasopressin V₁ₐ receptor than at one or more other related receptors, such as V_{1b}, V₂, or OT receptor.

Vasopressin V₁ₐ receptor antagonists that can be used in the methods of the invention include agents that are capable of crossing the blood brain barrier, and those that are not. Vasopressin V₁ₐ receptor antagonists that can be used in the methods of the invention include, but are not limited to, small molecule antagonists as well as biologics, such as antibodies, small interfering RNA (siRNA), microRNA, antisense nucleic acids, ribozymes, peptides and peptide analogues. Small molecule antagonists can be peptide or non-peptide compounds. Antibodies can be monoclonal antibodies or polyclonal antibodies that selectively bind to the vasopressin V₁ₐ receptor. More specifically, in one embodiment, antibodies that can be used in the methods of the invention are capable of inhibiting binding of arginine vasopressin to the vasopressin V₁ₐ receptor.

Examples of suitable small molecule vasopressin receptor V₁ₐ antagonists include, but are not limited to, SR49049 (Relcovaptan), atosiban (Tractocile®), conivaptan (YM-087), VPA-985, CL-385004, Vasotocin, SRX251 and SRX246 (Azevan), YM-218 (Astellas), OPC-2158 (Otsuka), and OPC21268.

In a particular embodiment, the vasopressin receptor V₁ₐ antagonist is 1-(beta-mercapto-beta beta-cycopentamethylenepropionic acid)-2-(O-methyl-Tyr)-argipressin, hereinafter referred to as “Compound 1,” or (CH2)5tyr(Me)AVP, having the structure:

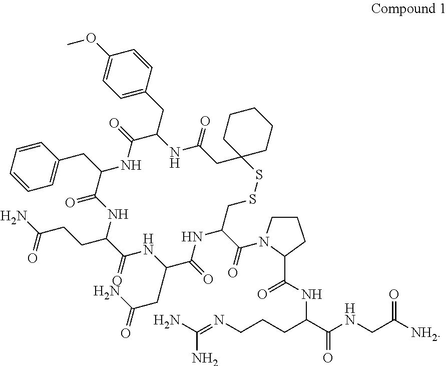 [SMILES: COc1ccc(CC2NC(=O)CC3(CCCCC3)SSCC(C(=O)N3CCCC3C(=O)NC(CCCN=C(N)N)C(=O)NCC(N)=O)NC(=O)C(CC(N)=O)NC(=O)C(CCC(N)=O)NC(=O)C(Cc3ccccc3)NC2=O)cc1]

Compound 1 may also be referred to as N-[1-[(2-amino-2-oxoethyl)amino]-5-(diaminomethylideneamino)-1-oxopentan-2-yl]-1-[13-(2-amino-2-oxoethyl)-16-(3-amino-3-oxopropyl)-19-benzyl-22-[(4-methoxyphenyl)methyl]-12,15,18,21,24-pentaoxo-7,8-dithia-11,14,17,20,23-pentazaspiro[5.19]pentacosane-10-carbonyl]pyrrolidine-2-carboxamide, (d(CH2)5(1)-O-Me-Tyr(2)-Arg(8))vasopressin, 1-(beta mercapto-beta,beta-cycopentamethylenepropionic acid)-2-O-methyltyrosyl-8-arginine vasopressin, 1-(beta-mercapto-beta beta-cyclpentamethylenepropionic acid)-2-(O-methyl-Tyr)-argipressin, AAVP, arginine vasopressin, beta-mercapto-(beta,beta-cyclopentamethylenepropionic acid)(1)-methyl-Tyr(2)-, argipressin, (beta-mercapto)beta,beta-cyclopentamethylenepropionic acid(1)-O-methyl-Tyr(2)-, AVPA, CGP 25838, CGP-25838, CPG 25838E, d(CH2)5(Tyr(Me)(2))AVP, d(CH2)5-Tyr(Me)argipressin, d(CH2)5Tyr(Me)AVP, Compound 1, MCPPA-AVP, Sigma V-2255, SK and F 100273, SK and F-100273, SKF 100273, [1-Mercaptocyclobhexyl]acetyl-Tyr[O-Methyl]-Phe-Gn-Asn-Cys-Pro-Arg-Gly-NH2 (SEQ ID NO: 21), 5.BDBM85096, CAS_105077, NSC_105077, or AM008588.

In other embodiments, the vasopressin receptor V₁ₐ antagonist is a position-modified variant of Compound 1 or is closely related analogue [1-deaminopenicillamine,2-O-methyltyrosine]arginine-vasopressin (dPTyr(Me)AVP). Some examples of such analogues can be found in Manning et al. J Med Chem. 1992, 25(2), 382-8, incorporated herein by reference.

The vasopressin V₁ₐ receptor antagonist can be administered per se as well as in the form of pharmaceutically acceptable esters, salts, and ethers, as well as other physiologically functional derivatives of such compounds. The vasopressin V₁ₐ receptor antagonist can be amorphous or polymorphic. The term “crystal polymorphs”, “polymorphs”, or “crystal forms” means crystal structures in which a compound (or a salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Examples of crystal lattice forms include, but are not limited to, cubic, isometric, tetragonal, orthorhombic, hexagonal, trigonal, triclinic, and monoclinic. Recrystallization solvent, rate of crystallization, storage temperature, and other factors may cause one crystal form to dominate. Crystal polymorphs of the compounds can be prepared by crystallization under different conditions.

Additionally, the vasopressin V₁ₐ receptor antagonist, for example, the salts of the compounds, can exist in either hydrated or unhydrated (the anhydrous) form or as solvates with other solvent molecules. “Solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water, the solvate formed is a hydrate; and if the solvent is alcohol, the solvate formed is an alcoholate. Hydrates are formed by the combination of one or more molecules of water with one molecule of the substance in which the water retains its molecular state as H₂O.

Non-limiting examples of hydrates include monohydrates, dihydrates, etc. Non-limiting examples of solvates include ethanol solvates, acetone solvates, etc.

Examples of pharmaceutically acceptable acid addition salts include those formed with inorganic acids such as hydrochloric acid, hydrobromic acid, sulfuric acid, nitric acid, phosphoric acid, and the like; as well as organic acids such as acetic acid, trifluoroacetic acid, propionic acid, hexanoic acid, cyclopentanepropionic acid, glycolic acid, pyruvic acid, lactic acid, oxalic acid, maleic acid, malonic acid, succinic acid, fumaric acid, tartaric acid, citric acid, benzoic acid, cinnamic acid, 3-(4-hydroxybenzoyl)benzoic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, p-toluenesulfonic acid, and salicylic acid and the like.

Examples of a pharmaceutically acceptable base addition salts include those formed when an acidic proton present in the parent compound is replaced by a metal ion, such as sodium, potassium, lithium, ammonium, calcium, magnesium, iron, zinc, copper, manganese, aluminum salts and the like. Preferable salts are the ammonium, potassium, sodium, calcium, and magnesium salts. Salts derived from pharmaceutically acceptable organic non-toxic bases include, but are not limited to, salts of primary, secondary, and tertiary amines, substituted amines including naturally occurring substituted amines, cyclic amines and basic ion exchange resins. Examples of organic bases include isopropylamine, trimethylamine, diethylamine, triethylamine, tripropylamine, ethanolamine, 2-dimethylaminocthanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, tromethamine, N-methylglucamine, polyamine resins, and the like.

Exemplary organic bases are isopropylamine, diethylamine, ethanolamine, trimethylamine, dicyclohexylamine, choline, and caffeine.

### Pharmaceutical Compostions Comprising Vasopressin V1a Receptor Antagonists

In various aspects of the invention, the vasopressin V₁ₐ receptor antagonist will be in the form of a pharmaceutical composition containing a pharmaceutically acceptable carrier. As used herein “pharmaceutically acceptable carrier” includes any and all solvents, dispersion media, coatings, antibacterial and antifungal agents, isotonic and absorption delaying agents, and the like that are physiologically compatible. In one embodiment, the pharmaceutically acceptable carrier is not phosphate buffered saline (PBS). In one embodiment, the carrier is suitable for intraocular, topical, parenteral, intravenous, intraperitoneal, or intramuscular administration. In another embodiment, the carrier is suitable for oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the vasopressin V₁ₐ receptor antagonist, use thereof in the pharmaceutical compositions of the invention is contemplated. Supplementary active compounds can also be incorporated into the compositions.

Oral Compositions

In the practice of the methods of the invention, the composition may be administered orally in any of the usual solid forms such as pills, tablets, capsules or powders, including sustained release preparations. The term unit dosage form as used in this specification and the claims refer to physically discrete units to be administered in single or multiple dosage to humans, each unit containing a predetermined quantity of active material, i.e., astaxanthin. The quantity of the vasopressin V₁ₐ receptor antagonist is calculated to produce the desired therapeutic effect upon administration of one or more of such units. It is understood that the exact treatment level will depend upon the case history of the human subject to be treated. The precise treatment level can be determined by one of ordinary skill in the art without undue experimentation, taking into consideration such factors as age, size, severity of condition, and anticipated duration of administration of compounds, among other factors known to those of ordinary skill.

Unit dosages can range from about 0.1 mg/kg to about 100 mg/kg of the vasopressin V₁ₐ receptor antagonist, preferably from about 10 mg/kg to about 30 mg/kg of the vasopressin V₁ₐ receptor antagonist, most preferably about 20 mg/kg of the vasopressin V₁ₐ receptor antagonist. The doses can be administered in any convenient dosing schedule to achieve the stated beneficial effects. For example, the doses can be taken 1, 2, 3, 4, 5 or more times daily, weekly, or bi-weekly. Most preferably, the doses are taken at meal times. The dosages may be taken orally in any suitable unit dosage form such as pills, tablets, and capsules.

Exemplary carriers for oral compositions include a solid or liquid filler, diluent, or encapsulating substance. Some examples of the substances that can act as carriers are sugars such as lactose, glucose, and sucrose; starches such as corn starch and potato starch; cellulose and its derivatives, such as sodium carboxymethylcellulose, ethylcellulose, cellulose acetate; powdered tragacanth; malt; gelatin; talc; stearic acid; magnesium stearate; calcium sulfate; vegetable oils, such as peanut oil, cottonseed oil, sesame oil, olive oil, corn oil and of the broma; polyols such as propylene glycol, glycerin, sorbitol, mannitol, and polyethylene glycol; agar, alginic acid; pyrogen-free water; isotonic saline; ethyl alcohol and phosphate buffer solutions, as well as other non-toxic compatible substances used in preparation of formulations. Wetting agents and lubricants such as sodium lauryl sulfate, as well as coloring agents, flavoring agents, and preservatives can also be present. Dye stuffs or pigments may be added to the tablets, for example, for identification or in order to characterize combinations of active doses.

Other preparations that can be used orally include push-fit capsules made of gelatin, as well as soft, sealed capsules made of gelatin and a plasticizer such as glycerol or sorbitol. The push-fit capsules can contain the active compounds in the form of granules, which may be mixed with fillers such as lactose, binders such as starches, and/or lubricants such as talc or magnesium stearate and, optionally, stabilizers. In soft capsules, the vasopressin V₁ₐ receptor antagonist is preferably dissolved or suspended in suitable liquids, such as fatty oils, or liquid paraffin. In addition, stabilizers may be added.

Powders are prepared by comminuting the compositions of the present invention to a suitable fine size and mixing with a similarly comminuted diluent pharmaceutical carrier such as an edible carbohydrate material as, for example, starch. Sweetening, flavoring, preservative, dispersing and coloring agents can also be present.

Capsules are made by preparing a powder mixture as described above and filling formed gelatin sheaths. A lubricant such as talc, magnesium stearate and calcium stearate can be added to the powder mixture as an adjuvant before the filling operation; a glidant such as colloidal silica can be added to improve flow properties; a disintegrating or solubilizing agent may be added to improve the availability of the medicament when the capsule is ingested.

Tablets are made by preparing a powder mixture, granulating or slugging, adding a lubricant and disintegrant and pressing into tablets. A powder mixture is prepared by mixing the compositions of the present invention, suitable comminuted, with a diluent or base such as starch, sucrose, kaolin, dicalcium phosphate, and the like. The powder mixture can be granulated by wetting with a binder such as syrup, starch paste, acacia mucilage or solutions of cellulosic or polymeric materials and forcing through a screen. As an alternative to granulating, the powder mixture can be run through the tablet machine and the resulting imperfectly formed slugs broken into granules. The granules can be lubricated to prevent sticking to the tablet forming dies by means of the addition of stearic acid, a stearate salt, talc or mineral oil. The lubricated mixture is then compressed into tablets. The vasopressin V₁ₐ receptor antagonist can also be combined with free flowing inert carriers and compressed into tablets directly without going through the granulating or slugging steps. A protective coating consisting of a sealing coat of shellac, a coating of sugar or polymeric material and a polish coating of wax can be provided. Dye stuffs or pigments may be added to the tablets, for example, for identification or in order to characterize combinations of active doses. In tablet form the carrier comprises from about 0.1% to 99.9% by weight of the total composition.

Oral compositions generally include an inert diluent or an edible carrier. They can be enclosed in gelatin capsules or compressed into tablets. For the purpose of oral therapeutic administration, the vasopressin V₁ₐ receptor antagonist can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic, acid, Primogel, or corn starch; a lubricant such as magnesium stearate or Sterotes; a glidant: such as colloidal silicon dioxide; a sweetening agent such as sucrose or saccharin; or a flavoring agent such as peppermint, methyl salicylate, or orange flavoring.

In one embodiment, the compositions of the invention are prepared with carriers that will protect the vasopressin V₁ₐ receptor antagonist against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These may be prepared according to methods known to those skilled in the art, for example, as described in U.S. Pat. No. 4,522,811.

Other Modes of Administration

In another embodiment, the pharmaceutical compositions of the present invention would be administered in the form of injectable compositions. The compositions can be prepared as an injectable, either as liquid solutions or suspensions. The preparation may also be emulsified. Suitable excipients are, for example, water, saline, dextrose, glycerol, ethanol, or the like and combinations thereof. In addition, if desired, the preparation may contain minor amounts of auxiliary substances such as wetting or emulsifying agents, pH-buffering agents, adjuvants, or immunopotentiators.

Sterile injectable solutions can be prepared by incorporating the compositions of the invention in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Nasal compositions generally include nasal sprays and inhalants. Nasal sprays and inhalants can contain one or more active components and excipients such as preservatives, viscosity modifiers, emulsifiers, buffering agents and the like. Nasal sprays may be applied to the nasal cavity for local and/or systemic use. Nasal sprays may be dispensed by a non-pressurized dispenser suitable for delivery of a metered dose of the active component. Nasal inhalants are intended for delivery to the lungs by oral inhalation for local and/or systemic use. Nasal inhalants may be dispensed by a closed container system for delivery of a metered dose of one or more active components.

In one embodiment, nasal inhalants are used with an aerosol. This is accomplished by preparing an aqueous aerosol, liposomal preparation or solid particles containing the compound. A non-aqueous (e.g., fluorocarbon propellant) suspension could be used. Sonic nebulizers may be used to minimize exposing the agent to shear, which can result in degradation of the compound.

Ordinarily, an aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the vasopressin V₁ₐ receptor antagonist can be formulated into ointments, salves, gels, or creams as generally known in the art.

The compositions of the invention can also be prepared in the form of suppositories (e.g., with conventional suppository bases such as cocoa butter and other glycerides) or retention enemas for rectal delivery.

Dosing

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of nucleic acid molecules described herein can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

Data obtained from cell culture assays and/or animal studies can be used in formulating a range of dosage for use in humans. The dosage typically will lie within a range of circulating concentrations that include the ED₅₀ with little or no toxicity. The dosage may vary within this range depending upon the dosage form employed and the route of administration utilized. The therapeutically effective dose of the vasopressin V₁ₐ receptor antagonist can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

In general, pharmaceutical compositions comprising the vasopressin V₁ₐ receptor antagonist for use in the methods of the invention can be administered in therapeutically effective amounts via any of the usual and acceptable modes known in the art, as described above, either singly or in combination with one or more therapeutic agents. A therapeutically effective amount can vary widely depending on the severity of the disease, the age and relative health of the subject, the potency of the compound used and other factors involved, as readily determinable within the skill of the art. Suitable therapeutic doses of the vasopressin V₁ₐ receptor antagonist may be in the range of 1 microgram (μg) to 1000 milligrams (mg) per kilogram body weight of the recipient per day, and any increment in between, such as, e.g., 1, 2, 3, 5, 7, 10, 15, 20, 25, 30, 35, 40, 45, 50, 75, 100, 200, 300, 400, 500, 600, 700, 800, 900, or 1000 μg (1 mg); 2, 3, 5, 10, 25, 50, 75, 100, 200, 300, 400, 500, 600, 700, 800, 900 or 1000 mg. Suitable therapeutic doses of the vasopressin V₁ₐ receptor antagonist may also be in the range of 1 μg to 200 μg, 1 μg to 150 μg, 1 μg to 100 μg, 1 μg to 50 μg, 5 μg to 200 μg, 5 μg to 100 μg, or 5 μg to 50 μg. The dose of vasopressin V₁ₐ receptor antagonist may be administered daily, every other day, weekly, bi-weekly, or monthly, as appropriate.

A desired dose can be presented as one, two, three, four, five, six, or more sub-doses administered at appropriate intervals throughout the day. These sub-doses can be administered in unit dosage forms, for example, containing from 1 μg to 1000 mg of active ingredient per unit dosage form. Alternatively, if the condition of the recipient so requires, the doses may be administered as a continuous infusion. The mode of administration and dosage forms will affect the therapeutic amounts of the compounds which are desirable and efficacious for the given treatment application.

It is understood, however, that a specific dose level for any particular subject will depend upon a variety of factors including the activity of the vasopressin V₁ₐ receptor antagonist, the age, body weight, general health, sex, diet, time of administration, rate of excretion, drug combination, and the severity of the particular disease being treated and form of administration.

These considerations, as well as effective formulations and administration procedures are well known in the art and are described in standard textbooks.

### Prognostic and Diagnostic Methods

Embodiments of the present invention relate to methods for diagnosing an eating disorder, including anorexia nervosa, including abnormal eating behavior, in a subject. In one particular embodiment, a method for diagnosing an eating disorder, including anorexia nervosa, or abnormal eating behavior, in a subject (or a predisposition therefore), is disclosed, wherein the method includes: (a) obtaining a biological sample from the subject; (b) determining a difference (e.g., increase) in the level of expression of AVP in the biological sample as compared to a control or reference sample; and (c) diagnosing an eating disorder, including anorexia nervosa, or abnormal eating behavior, in the subject, wherein a difference (increase) in the level of expression of the AVP biomarker correlates to a positive diagnosis of an eating disorder, including anorexia nervosa, in the subject.

In another aspect, the present invention also provides a method of determining whether a subject is a good candidate for treatment with a vasopressin V₁ₐ receptor antagonist, the method comprising the steps of determining the levels of arginine-vasopressin (AVP) in a biological sample obtained from the subject relative to the level of expression in a control sample, wherein increased expression of AVP indicates that the subject is a good candidate for treatment with a vasopressin V₁ₐ receptor antagonist.

In another aspect, the present invention also provides a method of determining whether a subject is a good candidate for treatment with a vasopressin V₁ₐ receptor antagonist, the method comprising the steps of determining the levels of arginine-vasopressin (AVP) in a biological sample obtained from the subject after administration of a hypertonic solution of saline relative to the level of expression in a control sample, wherein increased expression of AVP indicates that the subject is a good candidate for treatment with a vasopressin V₁ₐ receptor antagonist.

In yet another aspect, the invention provides a method for assessing the efficacy of a therapeutic agent for the treatment of an eating disorder, e.g., anorexia nervosa, in a subject, comprising the steps of (a) determining the levels of arginine-vasopressin (AVP) in a biological sample obtained from the subject, prior to therapy with the therapeutic agent; and (b) determining the levels of AVP in a biological sample obtained from the subject, at one or more time points during therapy with the therapeutic agent, wherein the therapy with the therapeutic agent is efficacious for treating the eating disorder in the subject when there is a lower level of AVP in the second or subsequent samples, relative to the first sample.

In still another aspect, the present invention also provides a method of determining whether a subject has or is at risk for developing an eating disorder, or is a good candidate for treatment with a vasopressin V₁ₐ receptor antagonist, the method comprising the steps of detecting expression of AVPR1A in the amygdala, wherein overexpression of AVPR1A in the amygdala relative to the level of expression in a control sample indicates that the subject has or is at risk for developing an eating disorder, and/or is a good candidate for treatment with a vasopressin V₁ₐ receptor antagonist. In one embodiment, expression of AVPR1A is detected using a PET ligand. In another embodiment, the PET ligand is a radiolabeled vasopressin V₁ₐ receptor antagonist. In a particular embodiment, the PET ligand comprises SRX246. (see Fabio et al. Bloorganic & Medicinal Chemistry 20, 1337-1345 (2012)).

In some embodiments, the control or reference sample can be obtained, for example, from a normal biological sample of the subject or from a non-diseased, healthy subject.

In one embodiment, the subject is a Val66Met carrier. In another embodiment, the subject is an individual having a history of past eating disorders. In another embodiment, the subject has a family history of eating disorders. In some embodiments, the subject has experienced intentional or unintentional weight loss, e.g., due to a calorie-restricting diet.

In one embodiment, the subject is an adolescent. In another embodiment, the subject is a female. In another embodiment, the subject is an adult that has experienced social stressors during adolescence. Social stressors during adolescence include, but are not limited to, physical and/or emotional abuse, isolation, displacement, and the like. In some embodiments, the subject has, or is at risk of having, anxiety or an anxiety disorder.

The biomarkers that can be used in the methods of the present invention include an AVP biomarker and an AVPR1A biomarker. In certain, non-limiting embodiments, more than one biomarker useful for the diagnosis of an eating disorder can be used in combination.

In one embodiment, a method for diagnosing an eating disorder in the subject includes obtaining at least one biological sample from the subject. In various embodiments the one or more biomarker can be detected in blood (including plasma or serum). The step of collecting a biological sample can be carried out either directly or indirectly by any suitable technique. For example, a blood sample from a subject can be carried out by phlebotomy or any other suitable technique, with the blood sample processed further to provide a serum sample or other suitable blood fraction.

In one embodiment, the information provided by the methods described herein can be used by the physician in determining the most effective course of treatment. An indication of a diagnosis of an eating disorder would be desirably considered in conjunction with clinical features of a subject's presentation to confirm a diagnosis, for example the psychological indicators of an eating disorder or physical symptoms, such as weight loss. A positive result showing increased expression of one or more of these biomarkers may be preceded or followed by one or more further diagnostic measure, for example, psychological testing, and/or one or more therapeutic measure to treat the eating disorder, such as treatment with an AVPR1A antagonist as described herein, alone or in combination with other methods of treatment.

### Biomarker Detection

A biomarker used in the methods of the invention can be identified in a biological sample using any method known in the art. Determining the presence of a biomarker, e.g., protein or degradation product thereof the presence of mRNA or pre-mRNA, or the presence of any biological molecule or product that is indicative of biomarker expression, or degradation product thereof; can be carried out for use in the methods of the invention by any method described herein or known in the art. In one embodiment, detection of the biomarker in the sample by a method described herein or known in the art transforms the sample, thereby allowing detection of the biomarker.

Protein Detection Techniques

Methods for the detection of protein biomarkers are well known to those skilled in the art, and include but are not limited to bead-based multiplexing technology, e.g., xMAP® technology (Luninex Corporation), microarrays, (e.g., protein microarrays), mass spectrometry techniques, 1-D or 2-D gel-based analysis systems, chromatography, enzyme linked immunosorbant assays (ELISAs), radioimmunoassays (RIA), enzyme immunoassays (EIA), western blotting, immunoprecipitation, and immunohistochemistry. These methods use antibodies, or antibody equivalents, to detect protein. Antibody arrays, beads, or protein chips can also be employed, see for example U.S. Patent Application Nos. 20030013208A1; 20020155493A1, 20030017515 and U.S. Pat. Nos. 6,329,209 and 6,365,418, herein incorporated by reference in their entirety. ELISA and RIA procedures can be conducted such that a biomarker standard is labeled (with a radioisotope such as ¹²⁵I or ³⁵S, or an assayable enzyme, such as horseradish peroxidase or alkaline phosphatase), and, together with the unlabeled sample, brought into contact with the corresponding antibody, whereon a second antibody is used to bind the first, and radioactivity or the immobilized enzyme assayed (competitive assay). Alternatively, the biomarker in the sample is allowed to react with the corresponding immobilized antibody, radioisotope or enzyme-labeled anti-biomarker antibody is allowed to react with the system, and radioactivity or the enzyme assayed (ELISA-sandwich assay). Other conventional methods can also be employed as suitable.

The above techniques can be conducted essentially as a “one-step” or “two-step” assay. A “one-step” assay involves contacting antigen with immobilized antibody and, without washing, contacting the mixture with labeled antibody. A “two-step” assay involves washing before contacting, the mixture with labeled antibody. Other conventional methods can also be employed as suitable.

In one embodiment, a method for measuring biomarker expression includes the steps of contacting a biological sample, e.g., blood, with a reagent, e.g., an anti-AVP antibody or variant (e.g., fragment) thereof which selectively binds the biomarker, thereby transforming the sample in a manner such that the level of expression of the biomarker is detected and quantified, e.g., by detecting whether the reagent is bound to the sample. A method can further include contacting the sample with a second reagent, e.g., antibody, e.g., a labeled antibody. The method can further include one or more steps of washing, e.g., to remove one or more reagents.

It can be desirable to immobilize one component of the assay system on a support, such as a bead, thereby allowing other components of the system to be brought into contact with the component and readily removed without laborious and time-consuming labor. It is possible for a second phase to be immobilized away from the first, but one phase is usually sufficient.

It is possible to immobilize the enzyme itself on a support, but if solid-phase enzyme is required, then this is generally best achieved by binding to antibody and affixing the antibody to a support, models and systems for which are well-known in the art.

Enzymes employable for labeling are not particularly limited, but can be selected from the members of the oxidase group, for example. These catalyze production of hydrogen peroxide by reaction with their substrates, and glucose oxidase is often used for its good stability, ease of availability and cheapness, as well as the ready availability of its substrate (glucose). Activity of the oxidase can be assayed by measuring the concentration of hydrogen peroxide formed after reaction of the enzyme-labeled antibody with the substrate under controlled conditions well-known in the art.

Other techniques can be used to detect a biomarker according to a practitioner's preference based upon the present invention. One such technique is western blotting (Towbin et al., Proc. Nat. Acad. Sci. 76:4350 (1979)), wherein a suitably treated sample is run on an SDS-PAGE gel before being transferred to a solid support, such as a nitrocellulose filter. Antibodies (unlabeled) are then brought into contact with the support and assayed by a secondary immunological reagent, such as labeled protein A or anti-immunoglobulin (suitable labels including ¹²⁵I, horseradish peroxidase and alkaline phosphatase). Chromatographic detection can also be used.

Other machine or autoimaging systems can also be used to measure immunostaining results for the biomarker. As used herein, “quantitative” immunohistochemistry refers to an automated method of scanning and scoring samples that have undergone immunohistochemistry, to identify and quantitate the presence of a specified biomarker, such as an antigen or other protein. The score given to the sample is a numerical representation of the intensity of the immunohistochemical staining of the sample, and represents the amount of target biomarker present in the sample. As used herein, Optical Density (OD) is a numerical score that represents intensity of staining. As used herein, semi-quantitative immunohistochemistry refers to scoring of immunohistochemical results by human eye, where a trained operator ranks results numerically (e.g., as 1, 2, or 3).

Various automated sample processing, scanning and analysis systems suitable for use with immunohistochemistry are available in the art. Such systems can include automated staining (see, e.g., the Benchmark system, Ventana Medical Systems, Inc.) and microscopic scanning, computerized image analysis, serial section comparison (to control for variation in the orientation and size of a sample), digital report generation, and archiving and tracking of samples (such as slides on which tissue sections are placed). Cellular imaging systems are commercially available that combine conventional light microscopes with digital image processing systems to perform quantitative analysis on cells and tissues, including immunostained samples. See, e.g., the CAS-200 system (Becton, Dickinson & Co.).

Antibodies against biomarkers can also be used for imaging purposes, for example, to detect the presence of a biomarker in a sample of a subject. Suitable labels include radioisotopes, iodine (¹²⁵, ¹²¹), carbon (¹⁴C), sulphur (³⁵S), tritium (³H), indium (¹¹²In), and technetium (^{99m}Tc), fluorescent labels, such as fluorescein and rhodamine and biotin. Immunoenzymatic interactions can be visualized using different enzymes such as peroxidase, alkaline phosphatase, or different chromogens such as DAB, AEC or Fast Red.

Antibodies and derivatives thereof that can be used encompasses polyclonal or monoclonal antibodies, chimeric, human, humanized, primatized (CDR-grafted), veneered or single-chain antibodies, phase produced antibodies (e.g., from phage display libraries), as well as functional binding fragments, of antibodies. For example, antibody fragments capable of binding to a biomarker, or portions thereof including, but not limited to Fv, Fab, Fab′ and F(ab′)2 fragments can be used. Such fragments can be produced by enzymatic cleavage or by recombinant techniques. For example, papain or pepsin cleavage can generate Fab or F(ab′)2 fragments, respectively. Other proteases with the requisite substrate specificity can also be used to generate Fab or F(ab′)2 fragments. Antibodies can also be produced in a variety of truncated forms using antibody genes in which one or more stop codons have been introduced upstream of the natural stop site. For example, a chimeric gene encoding a F(ab′)2 heavy chain portion can be designed to include DNA sequences encoding the CH, domain and hinge region of the heavy chain.

Synthetic and engineered antibodies are described in, e.g., Cabilly et al., U.S. Pat. No. 4,816,567 Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., European Patent No. 0451216 B1; and Padlan, E. A. et al, EP 0519596 A1. See also, Newman, R. et al, BioTechnology, 10: 1455-1460 (1992), regarding primatized antibody, and Ladner et al., U.S. Pat. No. 4,946,778 and Bird, R. E. et al., Science, 242: 423-426 (1988)) regarding single-chain antibodies.

In some embodiments, agents that specifically bind to a polypeptide other than antibodies are used, such as peptides. Peptides that specifically bind to AVP can be identified by any means known in the art, e.g., peptide phage display libraries. Generally, an agent that is capable of detecting a biomarker polypeptide, such that the presence of a biomarker is detected and/or quantitated, can be used. As defined herein, an “agent” refers to a substance that is capable of identifying or detecting a biomarker in a biological sample (e.g., identifies or detects the mRNA of a biomarker, the DNA of a biomarker, the protein of a biomarker). In one embodiment, the agent is a labeled or labelable antibody which specifically binds to a biomarker polypeptide.

In addition, a biomarker can be detected using Mass Spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.). See for example, U.S. Patent Application Nos: 20030199001, 20030134304, 20030077616, which are herein incorporated by reference.

Mass spectrometry methods are well known in the art and have been used to quantify and/or identify biomolecules, such as proteins (see, e.g., Li et al. (2000) Tibtech 18:151-160; Rowley et al. (2000) Methods 20: 383-397; and Kuster and Mann (1998) Curr. Opin. Structural Biol. 8: 393-400). Further, mass spectrometric techniques have been developed that permit at least partial de novo sequencing of isolated proteins. Chait et al., Science 262:89-92 (1993); Keough et al., Proc. Natl. Acad. Sci. USA. 96:7131-6 (1999); reviewed in Bergman, EXS 88:133-44 (2000).

In certain embodiments, a gas phase ion spectrophotometer is used. In other embodiments, laser-desorption/ionization mass spectrometry is used to analyze the sample. Modem laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”). See, e.g., U.S. Pat. No. 5,118,937 (Hillenkamp et al.), and U.S. Pat. No. 5,045,694 (Beavis & Chait). For additional information regarding mass spectrometers, see, e.g., Principles of Instrumental Analysis, 3rd edition. Skoog, Saunders College Publishing, Philadelphia, 1985; and Kirk-Othmer Encyclopedia of Chemical Technology, 4th ed. Vol. 15 (John Wiley & Sons, New York 1995), pp. 1071-1094.

Detection of the presence of a marker or other substances will typically involve detection of signal intensity. This, in turn, can reflect the quantity and character of a polypeptide bound to the substrate. For example, in certain embodiments, the signal strength of peak values from spectra of a first sample and a second sample can be compared (e.g., visually, by computer analysis etc.), to determine the relative amounts of a particular biomarker. Software programs such as the Biomarker Wizard program (Ciphergen Biosystems, Inc., Fremont, Calif.) can be used to aid in analyzing mass spectra. The mass spectrometers and their techniques are well known to those of skill in the art.

Any person skilled in the art understands, any of the components of a mass spectrometer (e.g., desorption source, mass analyzer, detect, etc.) and varied sample preparations can be combined with other suitable components or preparations described herein, or to those known in the art. For example, in some embodiments a control sample can contain heavy atoms (e.g., 13C) thereby permitting the test sample to be mixed with the known control sample in the same mass spectrometry run.

In some embodiments the relative amounts of one or more biomarkers present in a sample is determined, in part, by executing an algorithm with a programmable digital computer. The algorithm identifies at least one peak value in the first mass spectrum and the second mass spectrum. The algorithm then compares the signal strength of the peak value of the first mass spectrum to the signal strength of the peak value of the second mass spectrum of the mass spectrum. The relative signal strengths are an indication of the amount of the biomarker that is present in the first and second samples. A standard containing a known amount of a biomarker can be analyzed as the second sample to better quantify the amount of the biomarker present in the first sample. In certain embodiments, the identity of the biomarker in the first and second sample can also be determined.

PET imaging, as described herein, can also be used to detect a biomarker of the invention. For example, a PET ligand can be used to detect the presence or expression level of AVPR1A in a subject, e.g., in the amygdala.

RNA Detection Techniques

Any method for qualitatively or quantitatively detecting a nucleic acid biomarker can be used. Detection of RNA transcripts can be achieved, for example, by Northern blotting, wherein a preparation of RNA is run on a denaturing agarose gel, and transferred to a suitable support, such as activated cellulose, nitrocellulose or glass or nylon membranes. Radiolabeled cDNA or RNA is then hybridized to the preparation, washed and analyzed by autoradiography.

Detection of RNA transcripts can further be accomplished using amplification methods. For example, it is within the scope of the present disclosure to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap ligase chain reaction (RT-AGLCR) as described by R. L. Marshall, et al, PCR Methods and Applications 4: 80-84 (1994). In one embodiment, the sample being tested is transformed when the nucleic acid biomarker is detected, e.g., by Northern blotting or by amplification of the biomarker in the sample, in a manner such that the level of expression of the biomarker is detected and quantified.

In one embodiment, quantitative real-time polymerase chain reaction (qRT-PCR) is used to evaluate mRNA levels of biomarker. In one specific embodiment, the levels of one or more biomarkers can be quantitated in a biological sample.

Other known amplification methods which can be utilized herein include but are not limited to the so-called “NASBA” or “3SR” technique described in PNAS USA 87: 1874-1878 (1990) and also described in Nature 350 (No. 6313): 91-92 (1991); Q-beta amplification as described in published European Patent Application (EPA) No. 4544610; strand displacement amplification (as described in G. T. Walker et al, Clin. Chem. 42: 9-13 (1996) and European Patent Application No. 684315; and target mediated amplification, as described by PCT Publication WO9322461.

In situ hybridization visualization can also be employed, wherein a radioactively labeled antisense RNA probe is hybridized with a thin section of a biopsy sample, washed, cleaved with RNase and exposed to a sensitive emulsion for autoradiography. The samples can be stained with haematoxylin to demonstrate the histological composition of the sample, and dark field imaging with a suitable light filter shows the developed emulsion. Non-radioactive labels such as digoxigenin can also be used.

Another method for evaluation of biomarker expression is to detect mRNA levels of a biomarker by fluorescent in situ hybridization (FISH). FISH is a technique that can directly identify a specific region of DNA or RNA in a cell and therefore enables to visual determination of the biomarker expression in tissue samples. The FISH method has the advantages of a more objective scoring system and the presence of a built-in internal control consisting of the biomarker gene signals present in all non-neoplastic cells in the same sample. Fluorescence in situ hybridization is a direct in situ technique that is relatively rapid and sensitive. The FISH test also can be automated.

Alternatively, mRNA expression can be detected on a DNA array, chip or a microarray. Oligonucleotides corresponding to the biomarker(s) are immobilized on a chip which is then hybridized with labeled nucleic acids of a test sample obtained from a subject. Positive hybridization signal is obtained with the sample containing biomarker transcripts. Methods of preparing DNA arrays and their use are well known in the art. (see, for example, U.S. Pat. Nos. 6,618,6796; 6,379,897; 6,664,377; 6,451,536; 548,257; U.S. 20030157485 and Schena et al. 1995 Science 20:467-470; Gerhold et al. 1999 Trends in Biochem. Sci. 24, 168-173; and Lennon et al. 2000 Drug discovery Today 5: 59-65, which are herein incorporated by reference in their entirety). Serial Analysis of Gene Expression (SAGE) can also be performed (see, for example U.S. Patent Application 20030215858).

To monitor mRNA levels, for example, mRNA can be extracted from the biological sample to be tested, reverse transcribed and fluorescent-labeled cDNA probes are generated. The microarrays capable of hybridizing to a biomarker, cDNA can then probed with the labeled cDNA probes, the slides scanned and fluorescence intensity measured. This intensity correlates with the hybridization intensity and expression levels.

Types of probes for detection of RNA include cDNA, riboprobes, synthetic oligonucleotides and genomic probes. The type of probe used will generally be dictated by the particular situation, such as riboprobes for in situ hybridization, and cDNA for Northern blotting, for example. Most preferably, the probe is directed to nucleotide regions unique to the particular biomarker RNA. The probes can be as short as is required to differentially recognize the particular biomarker mRNA transcripts, and can be as short as, for example, 15 bases; however, probes of at least 17 bases, more preferably 18 bases and still more preferably 20 bases are preferred. Preferably, the primers and probes hybridize specifically under stringent conditions to a nucleic acid fragment having the nucleotide sequence corresponding to the target gene. As herein used, the term “stringent conditions” means hybridization will occur only if there is at least 95% and preferably at least 97% identity between the sequences.

The form of labeling of the probes can be any that is appropriate, such as the use of radioisotopes, for example, ³²P and ³⁵S. Labeling with radioisotopes can be achieved, whether the probe is synthesized chemically or biologically, by the use of suitably labeled bases.

### Kits

In non-limiting embodiments, the present invention provides for a kit for determining whether a subject has an eating disorder or whether a subject is a good candidate for treatment with an AVPR1A antagonist, comprising a means for detecting the biomarker of the invention. The invention further provides for kits for determining the efficacy of a therapy for treating an eating disorder in a subject.

Types of kits include, but are not limited to, bead-based multiplexing technology, e.g., xMAP® technology (Luninex Corporation), packaged probe and primer sets (e.g. TaqMan probe/primer sets), arrays/microarrays, biomarker-specific antibodies and beads, which further contain one or more probes, primers or other detection reagents for detecting one or more biomarkers of the present invention.

In other non-limiting embodiments, a kit can comprise at least one antibody for immunodetection of the biomarker(s) to be identified. Antibodies, both polyclonal and monoclonal, specific for a biomarker, can be prepared using conventional immunization techniques, as will be generally known to those of skill in the art. The immunodetection reagents of the kit can include detectable labels that are associated with, or linked to, the given antibody or antigen itself such detectable labels include, for example, chemiluminescent or fluorescent molecules (rhodamine, fluorescein, green fluorescent protein, luciferase, Cy3, Cy5, or ROX), radiolabels (3H, 35S, 32P, 14C, 131I) or enzymes (alkaline phosphatase, horseradish peroxidase).

In a further non-limiting embodiment, the biomarker-specific antibody can be provided bound to a solid support, such as a column matrix, an array, or well of a microtiter plate. Alternatively, the support can be provided as a separate element of the kit.

In a specific, non-limiting embodiment, a kit can comprise a pair of oligonucleotide primers suitable for polymerase chain reaction (PCR) or nucleic acid sequencing, for detecting one or more biomarker(s) to be identified. A pair of primers can comprise nucleotide sequences complementary to one or more biomarker of the invention. Alternatively, the complementary nucleotides can selectively hybridize to a specific region in close enough proximity 5′ and/or 3′ to the biomarker position to perform PCR and/or sequencing. Multiple biomarker-specific primers can be included in the kit to simultaneously assay large number of biomarkers. The kit can also comprise one or more polymerases, reverse transcriptase and nucleotide bases, wherein the nucleotide bases can be further detectably labeled.

In non-limiting embodiments, a primer can be at least about 10 nucleotides or at least about 15 nucleotides or at least about 20 nucleotides in length and/or up to about 200 nucleotides or up to about 150 nucleotides or up to about 100 nucleotides or up to about 75 nucleotides or up to about 50 nucleotides in length.

In a further non-limiting embodiment, the oligonucleotide primers can be immobilized on a solid surface or support, for example, on a nucleic acid microarray, wherein the position of each oligonucleotide primer bound to the solid surface or support is known and identifiable.

In certain non-limiting embodiments, a kit can comprise one or more reagents, e.g., primers, probes, microarrays, or antibodies suitable for detecting expression levels of markers.

A kit can further contain means for comparing the biomarker with a control or reference, and can include instructions for using the kit to detect the biomarker of interest. Specifically, the instructions describes that the increase in the level of expression biomarker, e.g., as compared to a control sample, is indicative that the subject has an eating disorder.

This invention is further illustrated by the following examples which should not be construed as limiting. The contents of all references, Appendices, GenBank Accession Numbers, patents and published patent applications cited throughout this application, as well as the Figures, are hereby incorporated by reference.

### EXAMPLES

### Materials and Methods

### Methods Summary

All mice were generated from intercrosses of hBDNF^{Val/Met} mice (see Cao, L., et al. Curr Biol 17, 911-921 (2007)), females were used for all experiments unless otherwise indicated. Animals were randomly assigned to groups to receive social isolation at 5 weeks and/or daily handling from 6-7 weeks and/or 20-30% caloric restriction at 7 weeks. Food intake and weight was assessed 3 times per week starting at 7 weeks of age. Expression analyses were performed on the ⅓-rostral part of the hypothalamus containing the PVH, PFC, hippocampus, amygdala and pituitary using real-time RT-PCR. All blood samples were collected via orbital sinus puncture on avertin-anesthetized animals. Experimenters were not blinded to the experimental groups of animals.

### Animals

All mice were generated from intercrosses of hBDNF^{Val/Met} mice (see Id.). Animals were housed in temperature controlled rooms at 21° C. and subject to a 12 hour light-dark cycle. Mice had ad libitum access to standard chow diet (Lab Diet: PicoLab Rodent Diet 5053) and water, unless otherwise indicated. Animals randomly assigned to be in the environmental stressor group were singly-housed from 5 weeks of age. Food hoppers were given to all singly-housed animals to monitor food intake at 6 weeks of age. Animals were randomly assigned to be either ad libitum fed or exposed to a 20-30% reduction in caloric intake for 10-11 consecutive days starting at 7 weeks of age. For the restricted group, 70-80% of the caloric intake of controls was provided in two daily allotments. Food intake and body weight were assessed 3 times per week starting at 7 weeks of age. Animals were excluded if they were singly-housed prior to 5 weeks of age or displayed abnormal feeding behaviors prior to 7 weeks of age. Adrenal glands were dissected after sacrifice and weighed to calculate adrenal:body weight ratio. Investigators were not blinded to groups.

### Genotyping

Initially, genotyping of the hBDNF locus was performed using PCR on DNA extracted from tail tips as described (Id.) using the primer set BDNF-F: 5′-TCCACCAGGTGAGAAGAGTGA-3′ (SEQ ID NO:5), and BDNF-R: 5′-GAGGCTCCAAAGGCACTTGA-3′ (SEQ ID NO:6), followed by restriction-enzyme analysis with BsaA1, which cleaves the Val allele.

### Locomotor Activity

Central versus peripheral locomotor activity was assessed through a photobeam-based activity monitoring system incorporated in the Indirect Calorimetry System combined with Feeding Monitor and TSE ActiMot system. Animals were allowed to acclimate for at least 24 hours to the room in which the apparatus was located. Anxiety-related behavior was assessed for the first 24 hours in the system in all groups except singly-housed hBDNF^{Met/?} females exposed to caloric restriction (GED), where activity was assessed for the first 24 hours following the initiation of caloric restriction (CR).

### Corticosterone

Baseline serum for corticosterone was collected from tail bleeds on minimally stressed animals at 10 am in unheparinized tubes and allowed to clot before centrifugation, decanting, and storage at −20° C. until use. Serum was analyzed for corticosterone content via RIA (MP Biomedicals).

### Gene Expression

Expression analyses were performed on the ⅓-rostral part of the hypothalamus containing the PVH, prefrontal cortex, hippocampus, amygdala and pituitary using real-time RT-PCR. Hypothalami and pituitary were quickly dissected, snap frozen in liquid nitrogen and stored at −80° C. until the mRNAs were extracted using RNeasy Plus Universal Mini Kit (Qiagen, Austin, Tex., USA) according to the manufacturer's guidelines. cDNAs were obtained from reverse transcription of total RNA using the transcriptor first-strand cDNA synthesis kit (Roche). Expression of arginine-vasopressin (AVP) (forward, GGGCATCTGCTGCAGCGACGAGAG (SEQ ID NO:7); reverse, AGCGCGGGTGAGGCGGAAAAA (SEQ ID NO:8)), arginine-vasopressin receptor 1a (Avpr1a) (forward, TCCCGTGCCAAGATCCGCACA (SEQ ID NO:9); reverse, GGTGGAAGGGTTTTCGGAATCGGT (SEQ ID NO: 10)), arginine-vasopressin receptor 1b (Avpr1b) (forward, TCTGTGTGGGACGAGAATGC (SEQ ID NO: 11); reverse GCGGTGACTCAGGGAACGT (SEQ ID NO:12)), Crh (forward, ATCTCACCTTCCACCTTCTGCG (SEQ ID NO: 13); reverse, CCCGATAATCTCCATCAGTTTCC (SEQ ID NO:14)) and Pomc (forward, AGTGCCAGGACCTCACCA (SEQ ID NO:15); reverse CAGCGAGAGGTCGAGTTTG (SEQ ID NO:16)); Oxtr (forward, TGTCTGGTCACTGTGCACTC (SEQ ID NO: 17); reverse CTAGGCI-TTTTGGAAGCGCC (SEQ ID NO: 18)) were quantified on a LightCycler (Roche) using the Light Cycler 480 SYBR Green I master (Roche).

Beta actin (forward, AAGGAAGGCTGGAAAAGAGC (SEQ ID NO:19); reverse, AAATCGTGCGTGACATCAA (SEQ ID NO:20)) was used as housekeeping gene.

Relative quantification was calculated using the 2-ΔΔCt formula where Ct is the cycle threshold at which the amplified PCR product was detected and 2-ΔΔCt represents the fold change in gene expression normalized to beta actin and relative to the control group.

### Serum Analysis

All blood samples were collected between 10 AM and noon via orbital sinus puncture on avertin-anesthetized animals. Blood was kept on ice before being centrifuged, plasma was decanted and stored at −80° C. before further analysis. AVP serum concentration was quantified using a specific Enzyme Immunoassay (EIA) (arg-Vasopressin EIA kit, ADI-900-017, Enzo Life Sciences, Framingdale, N.Y., USA) according to the manufacturer's protocol.

### Adolescent Handling

Animals exposed to peri-pubertal handling enrichment (singly-housed hBDNF^{Met/?} females (GEH), and singly-housed hBDNF^{Met/?} females exposed to caloric restriction (GEDH)) were held daily for ˜3 min from week 6 (P42) to week 7 (P49). Animals were returned to their home cages after handling. Animals that did not receive handling were not disturbed from week 6 to week 7.

### Statistics

The sample sizes in the present study were chosen based on common practice in animal behavior experiments (10-15 animals per group). Sample sizes in singly-housed hBDNF^{Met/?} females (GE) and GED groups were initially powered to permit separate analyses of homozygotes (hBDNFMet/Met) and heterozygotes (hBDNF^{Val/Met}). It was found that all phenotypes assessed were the same in hBDNF^{Met/Met} and hBDNF^{Met/Val} females (data not shown). Thus, we combined data from these genotypes into one group (hBDNF^{Met/?}) for statistical analyses, creating groups with larger samples sizes than the others (GE n=34 and GED n=36). We compared the average number of aphagic episode/animal in each group, as opposed to the percent of animals with aphagic episodes in each group, because it provided a parametric value for statistical analyses. Statistical comparisons were performed between groups using 2-tailed, unpaired Student's t test or 1-way ANOVA with Fisher's PLSD post hoc analysis. A P value of 0.05 or less was considered to be statistically significant. Data are presented as group mean±s.e.m.

### Example 1: BDNF (Val66Met) Variant and Stress Interact to Modulate AVPR1A Tone and Feeding Behavior

In this Example, anorexia-like behavior was elicited in mice by combining factors that are associated with increased risk of anorexia nervosa—adolescent females, genetic predisposition to anxiety imposed by the brain-derived growth factor (BDNF)-Val66Met gene variant, social stress and caloric restriction, thus developing a mouse model useful for identifying factors responsible for susceptibility to an anorexia nervosa-like behavior.

As described in detail below, using this novel mouse model, the inventors have found that Val66Met carriers exposed to social stress exhibit elevated levels of arginine-vasopressin (AVP) in the serum and increased expression of its receptor, Avpr1a, in the amygdala, before the onset of abnormal feeding behavior. Thus, elevated levels of AVP in the serum and increased expression of Avpr1a in the amygdala are correlated with subsequent risk for anorexic behavior, e.g., severe dietary restriction. Daily handling of the animals reversed increases in AVP tone and prevented aphagic behavior in this model. Thus, this study characterizes a “gene×environment” interaction that represents the underlying driver of abnormal eating behavior in certain anorexia nervosa patients, and also identifies AVPR1A as a target for therapeutic intervention.

Affective, anxiety and eating disorders share genetic, biological and psychosocial risk factors, consistent with common pathways to disease (Wade, et al. Am J Psychiatry 157, 469-471 (2000)). There is considerable overlap in symptoms, as affective disorders are often accompanied by changes in eating habits or body weight, and eating disorders are usually preceded by depression and/or anxiety and involve obsessive behaviors (Bulik, et al. Acta Psychiatr Scand 96, 101-107 (1997); Godart, et al., Eur Psychiatry 15, 38-45 (2000); Kaye, et al., Am J Psychiatry 161, 2215-2221 (2004)). Thus, psychotropic medications are the first line of treatment for eating disorder patients. While some of these compounds can ameliorate psychiatric symptoms, they do not impact life-threatening eating behaviors or weight loss (Herpertz, S., et al. Dtsch Arztebl Int 108, 678-685 (2011); Lebow, J., et al. Int J Eat Disord 46, 332-339 (2013); Harris, E. C. & Barraclough, B. Br J Psychiatry 173, 11-53 (1998)).

Brain derived neurotrophic factor (BDNF) plays an important role in the development of neuronal circuits regulating cognitive-, anxiety- and eating-related behaviors (Kernie, et al. Embo J 19, 1290-1300 (2000); Egan, et al. Cell 112, 257-269 (2003); Chen, Z. Y., et al Science 314, 140-143 (2006)). The common BDNF-Val66Met gene variant, characterized by impaired BDNF release and function (Egan, et al.; Chen et al.; Cao, L., et al. Curr Biol 17, 911-921 (2007)), is associated with anxiety and eating disorders (Ribases, M., et al. Eur J Hum Genet 13, 428-434 (2005); Akkermann, K., et al. Psychiatry Res 185, 39-43 (2011); Colzato, et al. Psychoneuroendocrinology 36, 1562-1569 (2011); Tocchetto, A., et al. Neurosci Left 502, 197-200 (2011)). The BDNFVal66Met variant plays a critical role in mediating the influence of early life stress on the severity of anxiety and depressive symptoms in adolescence (Aguilera, M., et al. Psychol Med 39, 1425-1432 (2009) and Perea, C. S., et al. J Affect Disord 136, 767-774 (2012)). Although this variant and preexisting anxiety are independently associated with increased severity of eating disorders (Ribases, M., et al.; Raney, T. J., et al. Int J Eat Disord 41, 326-332 (2008); Dellava, J. E., et al. Behav Res Ther 48, 60-67 (2010)), the relationship between genetic and environmental factors that promote abnormal eating behaviors, as described herein, has not previously been examined.

### Results

### “Gene (G)×Environment (E)” Interactions Promote Aphagic Behavior

The feeding-related behaviors in a mouse knock-in model of the hBDNF-Val66Met allele (Cao, L., et al.) were investigated to determine if these behaviors are similarly exacerbated by persistent exposure to social isolation stress from 5 weeks, an environmental condition reported to modulate stress axis responsivity, behavior, and body weight (see Workman et al. Pharmacol Biochem Behav 100, 25-32 (2011); Niwa, M., et al. Science 339, 335-339 (2013)). Initial efforts were focused on characterizing phenotypes in females, due to the increased prevalence of affective, anxiety and eating disorders (see Merikangas et al. J Am Acad Child Adolesc Psychiatry 49, 980-989 (2010)).

Four groups of mice were studied: hBDNF^{Val/Val} (without the susceptibility allele) maintained in group housing (control, CTL); hBDNF^{Met/?} (i.e., homozygous or heterozygous for the Met allele) maintained in group housing (genetic susceptibility, G); hBDNF^{Val/Val} singly-housed from 5 weeks of age (environmental stressor, E); and hBDNF^{Met/?} singly-housed from 5 weeks (genetic susceptibility and environmental stressor, GE) (Table 1).

TABLE 1
Experimental groups. Shaded boxes indicate manipulated factors
different from control (CTL) in peri-pubertal female mice.

| | | | | Animals with | |
|---|---|---|---|---|---|
| Group | n | Genetic (G) | Environmental (E) | AE (%) | Statistical analysis to GE |
| CTL | 10 | BDNF^{-Val/Val} | Group housing | 0 | Fisher's exact, 2-tail, p = 0.18 |
| G | 13 | BDNF^{-Met/?} | Group housing | 0 | Fisher's exact, 2-tail, p = 0.17 |
| E | 14 | BDNF^{-Val/Val} | Single housing | 7.1 | Fisher's exact, 2-tail, p = 0.41 |
| GE | 34 | BDNF^{-Met/?} | Single housing | 20.6 | N/A |

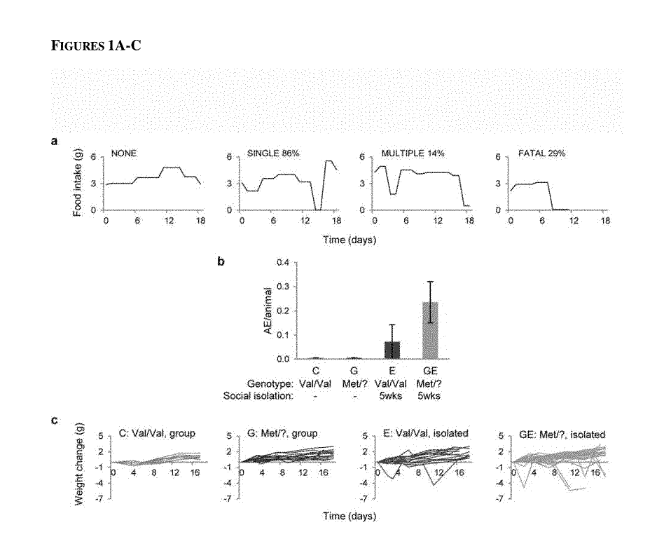

Food intake and body weight were measured in 7-week-old females that were allowed to acclimate to feeding from hoppers for one week. A subset of the experimental animals completely refrained from eating for extended periods of time. As daily food intake can fluctuate by 1.5 g and there is variability in measurements using hoppers, a threshold of consumption of <0.5 g food over 24 hours was established to quantify the incidence of “aphagic episodes (AE).” Aphagic episodes were typically followed by hyperphagia and a rebound to the initial body weight (FIG. 1A, “single”). However, failure to resume eating in 25% (n= 2/8) of aphagic mice was fatal (FIG. 1A, “fatal”). In addition, 14.3% of singly-housed hBDNF^{Met/?} Females (GE) exhibited at least one additional bout of aphagia in the period analyzed (FIG. 1A, “multiple”). Data from both genotypes was combined into one group (hBDNF^{Met/?}) for statistical analyses.

7.1% of hBDNF^{Val/Val} females exposed to social isolation stress (E) exhibited aphagic episodes, whereas no aphagic episodes were observed in group-housed hBDNF^{Val/Val} control (CTL) or hBDNF^{Met/?} (G) females (FIG. 1B, Table 1). Val66Met carriers exposed to social isolation stress (GE) exhibited a 3-fold increase in the prevalence of aphagic episodes compared to singly-housed hBDNF^{Val/Val} (E), although this difference did not reach significance (FIG. 1B, Table 1). Of those mice that did not eat for 24 hours, 87.5% remained aphagic for a second 24 hour period and lost significant (>15%) body weight (FIG. 1C).

### Dietary Restriction Increases the Likelihood of Abnormal Feeding Behavior

As discussed above, it was found that interactions between genetic factors and adolescent social stress increases the risk of abnormal eating behavior, and therefore the impact of direct manipulation of caloric intake on the feeding behaviors in Val66Met carriers was subsequently considered. Dieting behavior in adolescents usually precedes and has been proposed to act as a trigger of eating disorders (see Neumark-Sztainer et al. J Am Diet Assoc 106, 559-568 (2006)). Moreover, the Val66Met allele has been implicated in the effects of severe caloric restriction (CR) to promote unhealthy eating behaviors (see Akkermann et al. Psychiatry Res 185, 39-43 (2011)).

To explore this issue, singly-housed hBDNF^{Val/Val} (ED) or hBDNF^{Met/?} (GED) females were exposed to a mild dietary restriction (D=approximately 25% restriction of caloric intake) at 7 weeks (Table 2), by providing them with 70-80% of the caloric intake of ad libitum-fed controls for 11 days, referred to as ED and GED groups, respectively. Both short-term (within one week) and long-term effects on feeding behaviors were assessed. However, as mice in the present study do not necessarily eat all of their allotted food immediately, the effects of dietary restriction in group-housed mice were not assessed.

TABLE 2
Experimental groups. Shaded boxes indicate manipulated factors different from
control (CTL) in female peri-pubertal mice.

| | | | | | Animals | Statistical |
|---|---|---|---|---|---|---|
| | | | Environmental | | with AE | Analysis to |
| Group | n | Genetic (G) | (E) | Dieting (D) | (%) | GED |
| CTL | 10 | BDNF^{-Val/Val} | Group housing | Ad libitum Fed | 0 | Fisher's exact, |
| | | | | | | 2-tail, p = 0.019 |
| ED | 17 | BDNF^{-Val/Val} | Single housing | Caloric Restriction | 11.8 | Fisher's exact, |
| | | | | | | 2-tail, p = 0.019 |
| GED | 36 | BDNR^{-Met/?} | Single housing | Caloric Restriction | 41.7 | N/A |

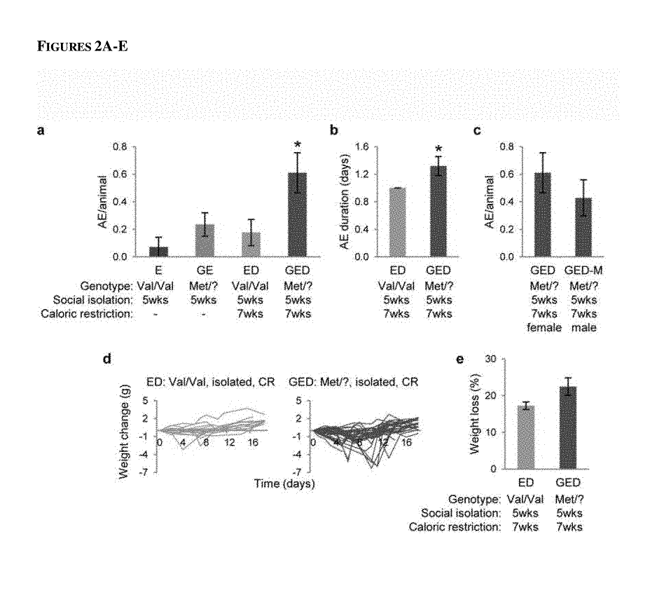

In the absence of genetic susceptibility factors, a trend toward increased incidence of aphagic episodes was observed in singly-housed hBDNF^{Val/Val} females subjected to caloric restriction as compared to those fed ad libitum (0.18 aphagic episode/animal in ED vs. 0.07 aphagic episode/animal in E) (FIG. 2A, Table 2), but this difference did not reach significance. Exposure to genetic, social and dietary risk factors was associated with a marked increase in the incidence of aphagic episodes compared to those groups exposed to any two risk factors (0.61 aphagic episode/animal in GED vs. 0.18 aphagic episode/animal in ED vs. 0.23 aphagic episode/animal in GE, P<0.05) (FIG. 2A, Table 2).

Segregation for the Val66Met allele was also associated with an increase in the severity of aphagic episodes, as reflected in the frequency of multiple aphagic episodes, the average length of an aphagic episode (1.3 day in GED vs. 1.0 day in ED, P<0.05), and severity of weight loss (21.9% in GED vs. 17.2% in ED) (FIG. 2B, D, E). Without being bound by any one particular theory, these data, in combination, indicate that dieting interacts with genetic and environmental risk factors to promote abnormal feeding behavior.

The frequency of aphagic episodes (aphagic episode/animal) in male Val66Met carriers that were exposed to adolescent social stress and caloric restriction (GED-M) was found to be 13% lower than in females, although this difference did not reach significance (FIG. 2C). There are two factors that could contribute to the apparent discrepancy between the instant findings and observations in anorexia nervosa. While the proportion of men with anorexia nervosa in clinical and case registry studies is only 10% (see Hock, H. W. Curr Opin Psychiatry 19, 389-394 (2006)), population-based studies estimate that the lifetime risk of anorexia nervosa is only three-fold lower in males (see Hudson et al. Biol Psychiatry 61, 348-358 (2007)), raising the possibility that there are more subclinical cases of anorexia nervosa in males that go unreported. In addition, it is possible that the preponderance of anorexia nervosa in females is driven by gender differences in behavior—namely the propensity to diet—rather than physiology. Depending on the metric considered, the prevalence of unhealthy dieting behaviors is 2-4 fold higher in females (see Drewnowski et al. Obes Res 2, 557-561 (1994)).

### Vulnerability to Peri-Pubertal Caloric Restriction

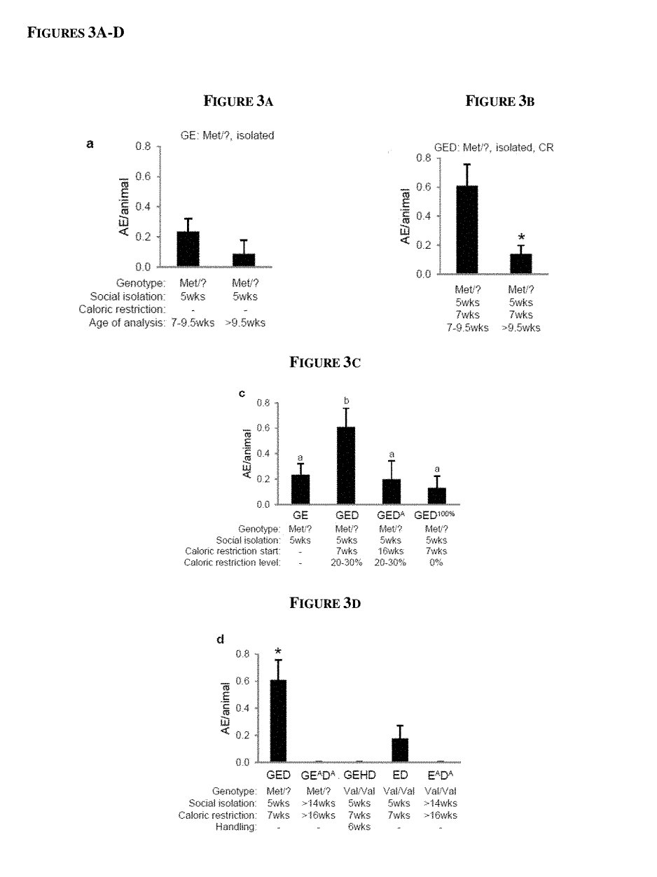

Eating disorders often emerge during adolescence after a period of intentional or unintentional weight loss (see Id.; Brandenburg et al. 12, 97-100 (2007)). Thus, the criticality of the timing of caloric restriction was examined. Food intake in females that were 7-9.5 weeks old versus >9.5 weeks old was compared. The prevalence of aphagic episodes was markedly increased in the younger singly-housed hBDNF^{Met/?} females with or without caloric restriction (GED and GE), although this difference only reached significance in the singly-housed Val66Met carriers under caloric restriction (GED) group (0.61 aphagic episode/animal in GED <9.5 weeks vs. 0.14 aphagic episode/animal in GED>9.5 weeks, P<0.05) (FIG. 3A,B). The lower incidence of aphagic episodes in GE and GED females older than 9.5 weeks was comparable to rates seen in young singly-housed hBDNF^{Val/Val} females (0.07 aphagic episode/animal in E<9.5 weeks) (FIG. 1B).

Next, the effect of exposing hBDNF^{Met/?} females that were singly-housed from 5 weeks to an 11-day caloric restriction in the peri-pubertal period (7 weeks=GED) was compared to adulthood (16 weeks of age=GED^{A}). It was observed that initiating caloric restriction at 7 weeks was more than three times as likely to elicit an aphagic episode as compared to caloric restriction at 16 weeks (0.61 aphagic episode/animal in GED vs. 0.2 aphagic episode/animal in GED^{A}, P<0.05) (FIG. 3C). Together, these findings indicate that interactions between the hBDNF^{Met/?} genotype and caloric restriction that increase the likelihood of abnormal feeding behaviors are most pronounced in the peri-pubertal period, similar to observations regarding anxiety-like behavior (see Bath et al. Biol Psychiatry (2012)).

Twice-daily feeding regimens are associated with changes in neuronal circuits regulating energy balance (see Knight et al. Cell 151, 1126-1137 (2012)), raising the possibility that psychological, rather than physiological responses to caloric restriction are acting in this model to promote abnormal feeding. To address this issue, body weight and food intake phenotypes were compared in singly-housed Val66Met carrier females that were subjected to a twice-daily feeding protocol at 7 weeks with 100% (i.e., no restriction) or 75% of the intake of ad libitum-fed controls (GED 100% and GED, respectively). It was found that the incidence of aphagic episodes in GED 100% females that had limited access to 100% of the daily caloric intake was 3-fold lower than was observed in GED females exposed to 25% caloric restriction (0.13 aphagic episode/animal in GED 100% vs. 0.61 aphagic episode/animal in GED, P<0.05) (FIG. 3C), and similar to that of singly-housed Val66Met carriers that were not exposed to caloric restriction at all (0.24 aphagic episode/animal in GE) (FIG. 3C). Without being bound by any one particular theory, these observations indicate that physiological cues associated with reduced caloric intake contribute to the risk of abnormal feeding behaviors in the instant model.

### Impact of Social Stress on Susceptibility to Anorexia Nervosa-Like Behavior are Conveyed During Adolescence

Social stressors experienced during adolescence have been reported to synergize with genetic factors to influence discrete neurochemical and behavioral deficits observed in some affective disorders (Id.). To explore whether early exposure to social isolation stress is critical to elicit abnormal feeding behaviors, the response to caloric restriction in adult females in which single housing was initiated at >14 weeks was compared with data at 5 weeks. A single aphagic episode was not observed in singly-housed hBDNF^{Val/Val} (E^{A}D^{A}) or hBDNF^{Met/?} (GE^{A}D^{A}) females in response to caloric restriction when exposure to social isolation stress was started in adulthood (n=8-10 per group, FIG. 3D). As the timing and nature of early life experiences dictate the penetrance and severity of anxiety and feeding endophenotypes observed in Val66Met carriers, they could underlie the modest and inconsistent effects reported for this variant in humans (see Dardennes et al. Psychoneuroendocrinology 32, 106-113 (2007); Hosang et al. BMC Med 12, 7 (2014)). Handling has been shown to reverse the effects of social isolation stress on some behavioral and neuronal endpoints (see Gentsch et al. Physiol Behav 43, 13-16 (1988); Rosa, M. L., et al. Braz J Med Biol Res 38, 1691-1696 (2005); Sciolino et al. Neuroscience 168, 371-386 (2010)). In singly-housed Val66Met carrier females that were handled ˜3 min every day for the week preceding caloric restriction (GEHD), not a single aphagic episode was observed (FIG. 3D).

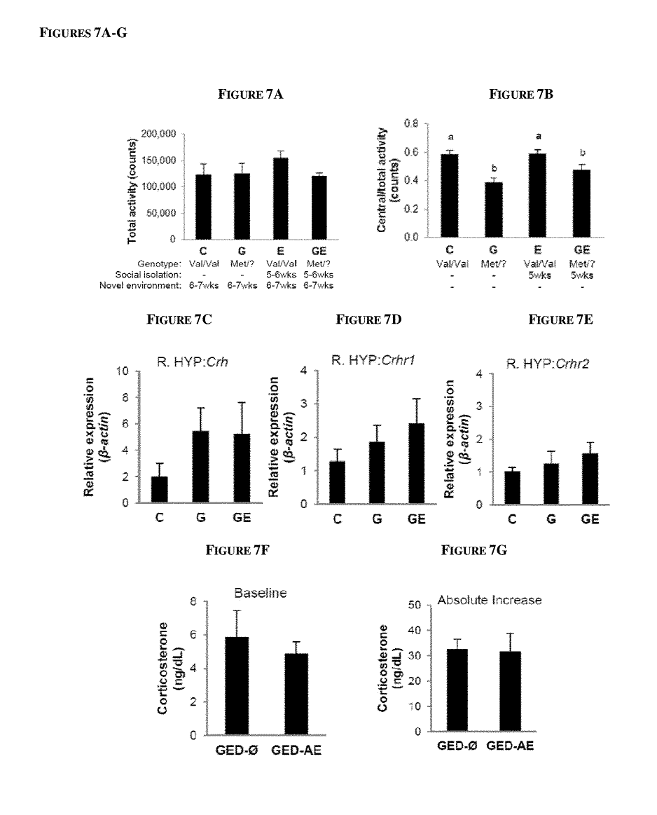

As the BDNF-Val66Met polymorphism has been associated with increased hypothalamus-pituitary-adrenal (HPA) axis reactivity and anxiety-like behaviors in mice and humans (see Id.; Chen et al. Science 314, 140-143 (2006); Vinberg et al. Psychoneuroendocrinology 34, 1380-1389 (2009); Yu et al. J Neurosci 32, 4092-4101 (2012), social isolation stress was examined to determine its effect on the exacerbation of these endophenotypes. Consistent with previous reports, increased anxiety-like behavior and a trend toward increased expression of the gene encoding corticotrophin releasing hormone (Crh) in the rostral third of the hypothalamus (which contains the paraventricular nucleus of the hypothalamus (PVH)) in 7-week-old Val66Met carriers (G) (FIGS. 7A-7E) was observed. However, social isolation did not appear to further magnify these phenotypes (FIG. 7A-7E). Moreover, there was no difference in baseline or restraint stress-induced serum corticosterone levels in 9.5-week-old, singly-housed hBDNF^{Met/?} females exposed to caloric restriction that had exhibited an aphagic episode (GED-aphagic episode) from those that did not (GED-Ø) (FIG. 7F and FIG. 7G). Together, these observations indicate that “gene×environment” interactions that promote abnormal feeding behavior in this model are not correlated with further exacerbations of anxiety-like behavior already imposed by the Val66Met genotype. (See Example 2, below).

Increased Serum Arginine-Vasopressin (AVP) and Pituitary Avpr1a Correlates with Anorexia Nervosa-Like Behavior

Elevated circulating levels of the neuropeptide arginine vasopressin (AVP) have been observed in patients with anorexia nervosa (see Gold et al. N Engl J Med 308, 1117-1123 (1983)), and the effect of peripheral injections of AVP to acutely suppress food intake is highly conserved across species (see Meyer et al. Q J Exp Physiol 74, 465-473 (1989); Ikemura et al. The Journal of veterinary medical science/the Japanese Society of Veterinary Science 66, 951-955 (2004)). Therefore, it was examined whether gene×environment interactions, as described herein, that promote abnormal feeding behavior in this model also impact AVP release into the circulation.

The fact that only a subset of age and exposure-matched GED females exhibited aphagic episodes by 9.5 weeks was used to identify changes in neuroendocrine and neuronal parameters that are signatures of aphagic behavior. Serum concentrations of AVP were found to be 2.5-fold higher in females that exhibited aphagic episodes (GED-aphagic episodes (AE)) than in those that did not (GED-Ø) (FIG. 4A). Elevated levels of circulating AVP were reflected in a 2.75-fold increase in Avp receptor 1a (Avpr1a) expression in the anterior pituitary of GED-aphagic episodes mice (FIG. 4B), while Avpr1b and Oxytocin receptor (Oxtr) expression were not different (FIG. 2B). Conversely, these increases in pituitary AVPR1A tone were reversed by the daily handling paradigm (FIG. 4A-C). Mice that were handled also exhibited reduced expression of pituitary Avpr1b (although this difference did not reach significance) (FIG. 4C), consistent with reports that this paradigm attenuates stress responsiveness (see Id.; Aguilera et al. Prog Brain Res 170, 29-39 (2008)).

### Impact of Social Isolation to Increase AVPR1A Tone in the Amygdala of Val66Met Carriers Precedes Feeding Phenotypes

The close association between serum AVP levels and risk of aphagic episodes at 9.5 weeks, raises the possibility that gene×environment interactions that raise serum AVP could precede and contribute to the vulnerability of some GED mice to caloric restriction. To explore this issue, circulating levels of AVP and/or pituitary expression of Avpr expression were evaluated to determine whether their levels were higher in 7-week-old singly-housed hBDNF^{Met/?} females (GE) compared to the other groups. Some GE females exhibited Avp levels that were roughly two-fold higher than in group-housed hBDNF^{Val/Val} (C) and hBDNF^{Met/?} (G) females (FIG. 5A). However, differences in pituitary expression of Avpr1a or Avpr1b (FIG. 5B, C) were not detected. The similarity between the percent of singly-housed hBDNF^{Met/?} females that exhibited aphagic episodes in response to caloric restriction (42%, n= 15/36) and those with serum AVP levels that were two-fold higher than controls (38%, n=⅜) is consistent with the idea that elevated serum AVP is predictive of individuals that will have adverse responses to caloric restriction, although terminal analyses required for this study did not permit this determination.

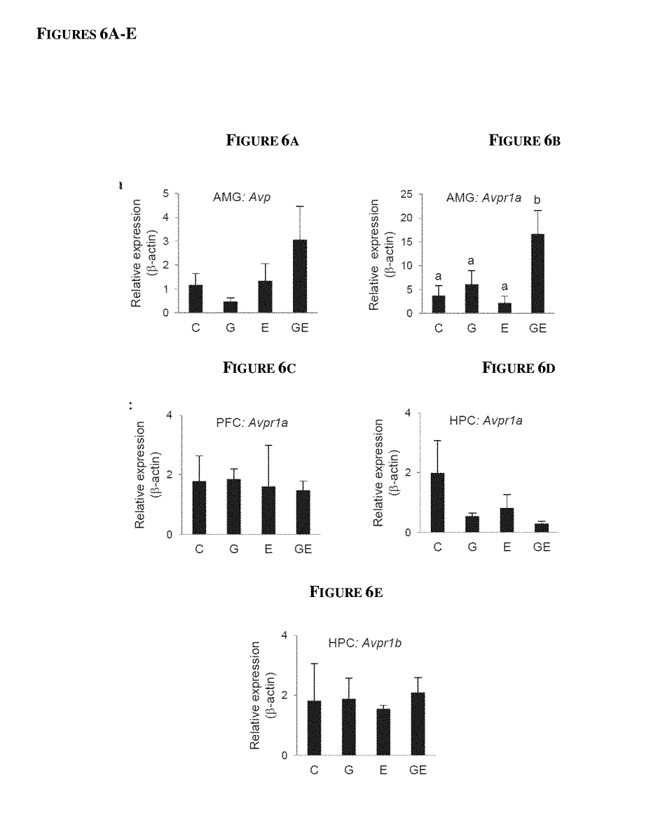

Since differences in Avpr expression in the pituitary in singly housed hBDNF^{Met/?} females (GE) at 7 weeks were not observed, central targets of AVP signals, which have also been implicated in mediating anorexigenic effects of peripheral AVP, were investigated (see Ikemura et al. The Journal of veterinary medical science/the Japanese Society of Veterinary Science 66, 951-955 (2004)). To this end, Avp and Avpr expression was assessed in three brain regions that are sensitive to social stressors during adolescence because they undergo remodeling during this period—the amygdala, hippocampus and prefrontal cortex (see Eiland et al. Neuroscience 249, 162-171 (2013)). Exposure of Val66Met carriers to social isolation stress was observed to be associated with 6-8-fold increases in Avp and Avpr1a expression in the amygdala (although the former did not reach significance), while Avpr1b was not expressed (FIG. 6A, B). In contrast, differences in the expression of genes encoding AVP receptors in the hippocampus (Avpr1a and Avpr1b) or prefrontal cortex (Avpr1a) were not detected; Avp is not expressed in either region (FIG. 6C-E).

The foregoing observation that increased AVPR1A tone in the amygdala of hBDNF^{Met/?} females exposed to social stress correlates with susceptibility to pathological eating behavior is consistent with a report that higher threat-related activity in the amygdala is predictive of later vulnerability to stress in humans (see Swartz et al. Neuron 85, 505-511 (2015)).

### Discussion

The inventors have shown that interactions between the Val66Met genotype and exposure to adolescent social stress that amplify AVPR1A tone in the amygdala increase the likelihood of anorexia nervosa-like behavior triggered by dieting. Since anti-depressants are reported to increase circulating levels of AVP (see Aravich et al. Brain Res 612, 180-189 (1993); de Magalhaes-Nunes et al. Exp Physiol 92, 913-922 (2007), the aforementioned supposition could also explain why these compounds do not improve (and sometimes aggravate) eating behavior or body weight in anorexia nervosa patients (see Herpertz et al. Dtsch Arztebl Int 108, 678-685 (2011); Lebow et al. Int J Eat Disord 46, 332-339 (2013)).

The results described herein indicate that developmental impacts on the AVP system can be exploited to develop novel serum biomarkers and therapeutic compounds to prevent and/or treat anorexia nervosa.

### Example 2: Social Stress and Dietary Restriction in Adolescence Promotes Susceptibility to Anorexic Behavior in BDNF-Val66Met Variant Mice

### Anorexic Behavior is not Due to an Exacerbation of Anxiety-Like Behavior

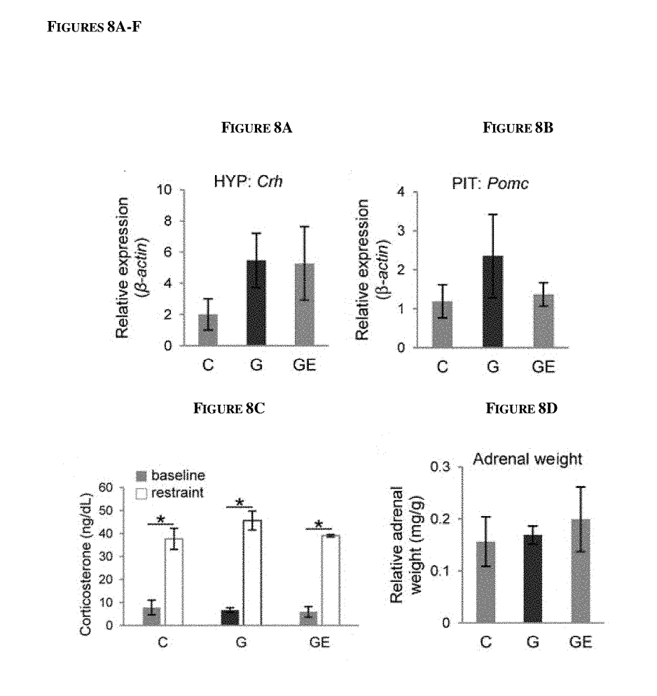

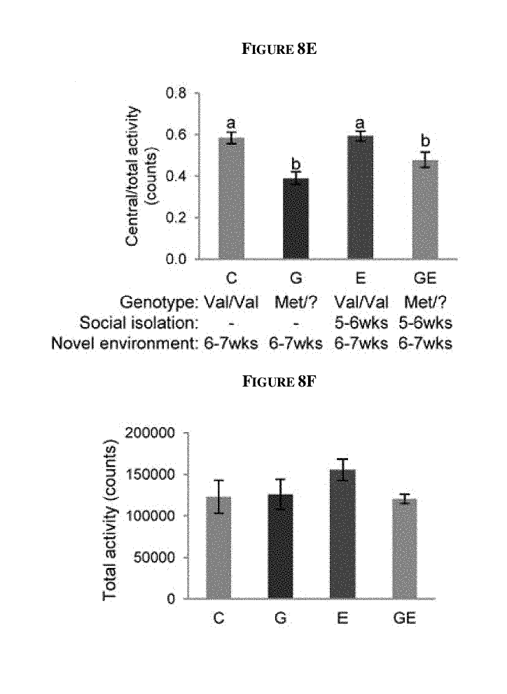

In this example, a combination of molecular, neuroendocrine and physiological criteria were used to evaluate hypothalamus-pituitary-adrenal (HPA) axis function: expression of genes encoding corticotrophin releasing hormone (Crh) in the rostral hypothalamus (which contains the paraventricular nucleus of the hypothalamus (PVH)) and Proopiomelanocortin (Pomc) in the pituitary, serum levels of the stress hormone CORT at baseline and in response to restraint stress, and adrenal gland weights. These studies suggest that Crh and Pomc expression in group-housed Val66Met carriers (G) at 7 weeks of age were more than 2-fold higher than in controls, although this difference did not reach significance (FIGS. 8A, B). Social isolation (GE) did not amplify these phenotypes (FIGS. 8A,B). Significant effects of the Val66Met genotype (G) or social isolation (GE) on acute and chronic measures of HPA axis activity were not detected in any of the groups (FIGS. 8C, D). Mice in all groups (C, G, GE) exhibited similar adrenal weights (FIG. 8D), and corticosterone (CORT) levels at baseline and in response to restraint stress (FIG. 8C). Consistent with previous reports (Chen Z Y, Jing D, Bath K G, Ieraci A, Khan T, Siao C J, et al. Science. 2006; 314(5796): 1403), Val66Met carriers appeared to exhibit increased anxiety-like behavior (0.39 central/total activity counts in G vs. 0.58 central/total activity counts in C, P<0.05); however this was not further exacerbated by social isolation (0.48 central/total activity counts in GE) (FIGS. 8E, F).

Without being bound by any one particular theory, these observations taken together support the conclusion that gene×environment interactions that promote abnormal feeding behavior in the model described herein are not correlated with further exacerbations of anxiety-like behavior already imposed by the hBDNF-Val66Met genotype.

### Adolescent Handling Prevents Anorexic Behavior

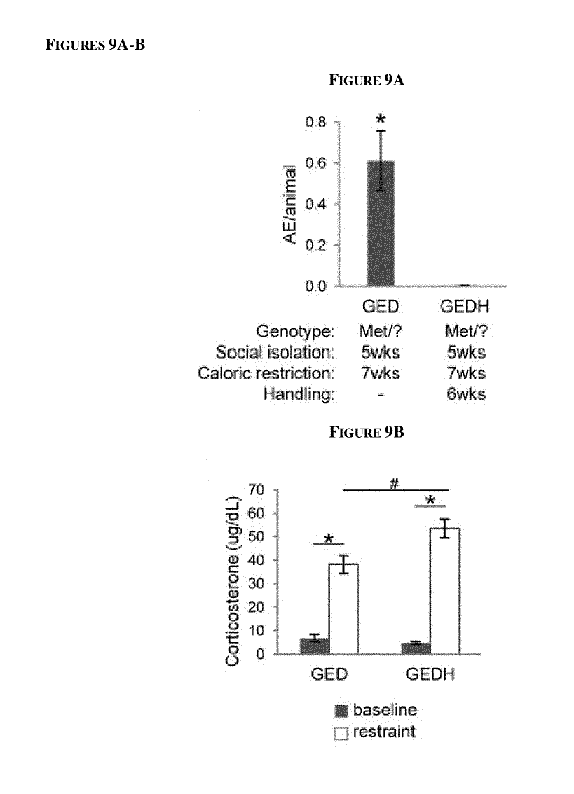

Handling has been shown to reverse the effects of social isolation stress on some behavioral and neuronal endpoints (Gentsch C, Lichtsteiner M, Frischknecht H R, Feer H, Siegfried B. Physiol Behav. 1988; 43(1):13-6; Rosa M L, Silva R C, Moura-de-Carvalho F T, Brandao M L, Guimaraes F S, Del Bel E A. Braz J Med Biol Res. 2005; 38(11):1691-6; Sciolino N R, Bortolato M, Eisenstein S A, Fu J, Oveisi F, Hohmann A G, et al. Neuroscience. 2010; 168(2):371-86). In singly-housed Val66Met carrier females that were handled for approximately 3 min every day for the week preceding caloric restriction (CR) (GEDH), no severe self-imposed dietary restriction, referred to herein as “aphagic episode” (AE) (FIG. 9A) was observed. Accordingly, it was explored whether the beneficial effects of daily handling might be mediated via reductions in the neuroendocrine response to psychological stress. To explore this, the response to immobilization for 15 minutes at 9.5 weeks, after release from CR was also assessed. Daily handling of GED females from 6 to 7 weeks did not affect CORT levels at baseline (6.7 ug/dL in GED vs. 4.6 ug/dL in GEDH p=0.23), but was unexpectedly associated with an elevated stress response at the end of the restraint period (38.2 ug/dL in GED vs. 53.5 ug/dL in GEDH p<0.05) (FIG. 9B). Without being bound by any one particular theory, these findings suggest against the possibility that the effect of handling to prevent anorexic behavior in GEDH mice is mediated through diminutions in HPA responsiveness.

### Discussion

A model to test the hypothesis that the Val66Met allele increases the likelihood of AN by conferring sensitivity to environmental factors was developed. The data presented in this experiment indicates that the Val66Met genotype promotes anorexic behavior in mice exposed to social isolation stress and caloric restriction during adolescence, but not when these environmental variables are imposed in adulthood. Gene×environment impacts on HPA axis function and anxiety-like behaviors were discordant with the incidence of anorexic behavior.

### A Novel Mouse Model to Study Triggers of AN

Approaches involving genetic, environmental and/or dietary manipulations have been used by other labs to study anorexia nervosa in animal models. The anx/anx mouse strain carries a genetic mutation that leads to dramatic reductions in food intake and body weight from the postnatal period (Ong K K, Northstone K, Wells J C K Plos Medicine. 2007; 4(4):737-42), which is a notable contrast to the common adolescent age of onset in humans (Hoek H W. Curr Opin Psychiatry. 2006; 19(4):389-945; 21). Genetically-engineered mouse strains have been used to ablate or activate distinct neuronal populations, with dramatic effects to suppress food intake (Luquet S, Perez F A, Hnasko T S, Palmiter R D. Science. 2005; 310(5748):683-5; Cai H, Haubensak W, Anthony T E, Anderson D J. Nat Neurosci. 2014; Lim B K, Huang K W, Grueter B A, Rothwell P E, Malenka R C. Nature. 2012; 487(7406):183-9). While these models have yielded insights into circuits that cause anorexia per se, it is not clear whether any given circuit contributes to the pathophysiology of AN in humans. Exposure to chronic and/or severe psychological stress directly suppresses food intake (Calvez J, Fromentin G, Nadkarni N, Darcel N, Even P, Tome D, et al. Physiol Behav. 2011; 104(5):675-83), however, the proximity of the timing and severity of the stressor are very different from the conditions that increase the risk of AN in humans (Kaye W H, Bulik C M, Thornton L, Barbarich N, Masters K. Am J Psychiatry. 2004; 161(12):2215-21; Godart N T, Flament M F, Lecrubier Y, Jeammet P. Eur Psychiatry. 2000; 15(1):38-45). A well-characterized animal model of AN is the activity-based anorexia model, which involves self-imposed starvation in response to exposure to a combination of restricted access to food and exercise (Routtenberg A, Kuznes of A W. J Comp Physiol Psychol. 1967; 64(3):414-21). While these models have provided insights into neuropeptide and neuronal pathways responsible for food intake suppression, they have not yielded insights into the triggers of AN in humans.

Several key aspects of self-imposed aphagic episodes in the mouse model described herein accurately reflect the conditions thought to promote Eating Disorders: 1) interactions between early life stress and the BDNF-Val66Met genotype increase susceptibility; 2) onset is often preceded by dieting; and 3) peak incidence in adolescence.

While the mouse model described herein recapitulates many of the risk factors associated with susceptibility to AN (Hoek H W. Curr Opin Psychiatry. 2006; 19(4):389-94; Kaye W H, Bulik C M, Thornton L, Barbarich N, Masters K. Am J Psychiatry. 2004; 161(12):2215-21; Strober M, Freeman R, Lampert C, Diamond J. Int J Eat Disord. 2007; 40 Suppl: S46-51; Bulik C M, Sullivan P F, Tozzi F, Furberg H, Lichtenstein P, Pedersen N L. Arch Gen Psychiatry. 2006; 63(3):305-12; Godart N T, Flament M F, Lecrubier Y, Jeammet P. Eur Psychiatry. 2000; 15(1):38-45; Lantzouni E, Frank G R, Golden N H, Shenker R I. J Adolesc Health. 2002; 31(2):162-5), there are two notable differences—lack of chronicity of self-imposed caloric restriction and diminished gender preference. During an aphagic episode in mice, food intake is suppressed by more than 80%. Thus, mice that maintain AEs for 3 days do not survive. Because the degree of food intake restriction is less severe in humans, this behavior can be maintained over a long period of time. It has been proposed that dieting and weight loss become a rewarding habit in some individuals, which fosters the persistence of this self-destructive behavior (Walsh B T. Am J Psychiatry. 2013; 170(5):477-84). The observations in this study are consistent with the idea that circuits responsible for triggering restrictive feeding behavior may be distinct from those that maintain it over a long period of time.

There are two factors that could contribute to the apparent discrepancy between the modest increase in AE prevalence in females in the model described herein (FIG. 2C) and epidemiological observations that males comprise only 10% of the AN patients in clinical and case registry studies (Hoek H W. Curr Opin Psychiatry. 2006; 19(4):389-94). First, population-based studies estimate that the lifetime risk of AN is only three-fold lower in males (Hudson J I, Hiripi E, Pope H G, Jr., Kessler R C. Biol Psychiatry. 2007; 61(3):348-58), supporting the idea that there are more subclinical cases of AN in males that go unreported. In addition, it is possible that the preponderance of AN in females is driven by gender differences in behavior—namely the propensity to diet—rather than physiology. Depending on the metric considered, the prevalence of unhealthy dieting behaviors is 2-4 fold higher in females (Drewnowski A, Kurth C L, Krahn D D. Obes Res. 1994; 2(6):557-61). As it was observed that CR increases the likelihood of AN by 2-fold, the lower prevalence of dieting in adolescent boys could account for their reduced risk of AN.

### Implications for Efforts to Treat AN

Of the eleven experimental groups studied, two conditions were associated with resistance to anorexic behavior—group housing during adolescence (control and G groups, FIG. 1B) or daily handling in singly-housed adolescents (GEDH, FIG. 9A). Notably, both group-housed Val66Met carriers (G group) and handled singly-housed Val66Met carriers (GEDH group) exhibited increased behavioral (FIG. 8E) and neuroendocrine (FIG. 9B) responses to psychological stress. These observations are consistent with reports that enrichment paradigms improve behavioral responses in mice exposed to early life stress (Hellemans K G, Benge L C, Olmstead M C. Brain Res Dev 2004; 150(2):103-15). While the consequences of daily handling during the postnatal period have been described, less is known about the impacts during adolescence. Pharmacological treatments can reverse the impacts of adolescent social stress on the neuroanatomical organization of circuits in the prefrontal cortex (Leussis M P, Lawson K, Stone K, Andersen S L. Synapse. 2008; 62(3):185-92). To explore whether compounds that modulate neurotransmitter activity might be used as therapies for AN, it is critical to identify the circuits responsible for anorexic behavior in the model described herein. The findings presented herein indicate that behavioral or pharmacological therapeutic strategies aimed at mitigating antecedent exposure to juvenile stress, rather than co-morbid psychiatric symptoms, can prevent and treat eating disorders.

### Conclusion

In conclusion, and without being bound by any one particular theory, the findings presented in this experiment indicate that exposure to juvenile social stress in genetically predisposed individuals increases the likelihood that caloric restriction in the peri-pubertal period will elicit anorexic behavior independent of effects on anxiety-related endpoints.

### Example 3: In Vivo Administration of an AVPR1A Antagonist to GED Mice

Based on the data described herein, increased AVPR1A signaling in the amygdala contributes to anorexic behavior, thus, AVPR1A antagonists can be used to treat AN. Therefore, the following study was illustrates that peripheral injection of an AVPR1A antagonist that can cross the blood brain barrier, Compound 1 (Kruszynski, M., et al, J Med Chem., 1980. 23(4): p. 3564-8; Manning, M., et al., J Neuroendocrinol, 2012. 24(4): p. 609-28), can increase food intake during an aphagic episode (AE).

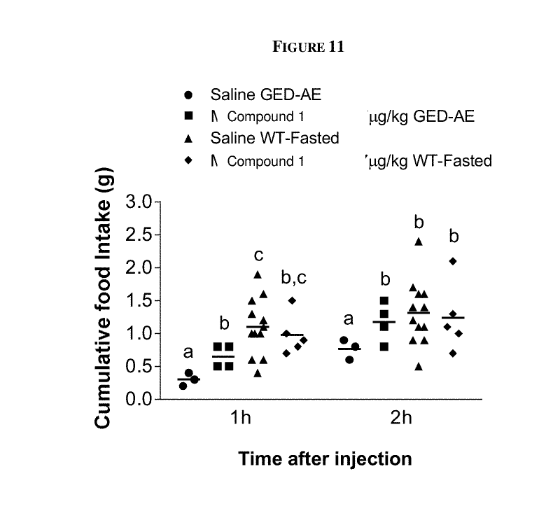

In the morning of the first day of an aphagic episode (≧18 hr of aphagia), GED mice were injected i.p. with 7 μg/kg Compound 1 (Sigma) vs. saline. GED mice injected with Compound 1 during an aphagic episode consumed significantly more food 1 hour (0.65g±0.09 vs. 0.3g±0.06) and 2 hours (1.18g±0.15 vs. 0.77g±0.09) after the injection (square vs. circle data points, FIG. 11).

To determine the extent to which treatment with Compound 1 rescued the normal drive to re-feed after overnight fast, food intake in a group of saline-injected GED mice that were eating normally were also measured, and were then subjected to an investigator-initiated overnight fast (a condition when AVPR1A should not be elevated in the amygdala) (triangle data points, FIG. 11).

This study reveals that the 7 μg/kg dose of Compound 1 can achieve a partial rescue of food intake at 1 hour, while 2 hour intake is nearly normalized (square vs. triangle data points in FIG. 11). Importantly, it was found that AVPR1A antagonists did not increase food intake in wild-type mice subjected to an overnight fast, consistent with the idea that their effect specific to situations where AVPR1A is hyperactive.

It was also investigated whether Compound 1 acts as a generic appetite stimulant. To test this idea, the impact of injecting Compound 1 after an overnight fast in wild-type mice at 7-8 weeks of age (FIG. 11, diamond data points) was tested. It was found that Compound 1 did not impact food intake in wild-type animals (FIG. 11, triangle vs. diamond data points). Thus, Compound 1 is not a generic appetite stimulant and its effect is specific to the anorexic model Thus, this study indicates that increased AVPR1A signal is driving the anorexic behavior in the model described herein and that AVPR1A antagonists can be used to treat AN.

### Example 4: Development of a PET Imaging Radioligand for AVPR1A

### Biodistribution of [¹⁸F]SRX246

SRX246-based radiotracers will be synthesized according to known methods (see Fabio et al. Bioorganic & Medicinal Chemistry 20, 1337-1345 (2012)). The synthesis of these compounds will be first performed in the “cold” (i.e., non-radioactive) laboratory followed by radiolabeling in the “hot”, radiochemistry laboratory. The radioligand will first be tested in mouse brain and pituitary, sites of abundant AVPR1A expression. The kidney, which expresses AVPR2, but not AVPR1A or AVPR1B, will serve as a control Female C57BL6/J mice at 7 weeks of age will be injected via the tail vein with 200-400 μCi (1 μg/Kg) of [¹⁸F]SRX246 in 0.2 mL of saline:sodium bicarbonate (7:3). Three mice for each time point will be sacrificed by cervical dislocation at 5, 15, 30, 60, and 90 minutes post injection. Mice will be anesthetized with isofluorane, decapitated and the brain regions, pituitary, and kidney quickly removed. The amygdala, hippocampus, medial prefrontal cortex, and lateral septum/bed nucleus of the stria terminals will be dissected as described (Zapala, et al. PNAS 102(29), 10357-62 (2005)). The tissue radioactivity will be measured with an automated scintillation counter. The percent injected dose per gram tissue will be calculated by comparison with samples of a standard dilution of the initial dose. All measurements will be corrected for radioactive decay of the radioligand itself.

### Binding Saturation

The saturability of [¹⁸F]SRX246 binding will be determined by pretreating mice intravenously with 0.01, 0.1, 0.2, and 0.3 mg/Kg of cold carrier SRX246 5 minutes prior to the injection of 0.2 mL of the [¹⁸F]SRX246 solution (0.001 mg/Kg; 200-400 μCi). Pretreatment with normal saline will be used as the control for this experiment. Three mice for each concentration (including no carrier added) will be sacrificed at 30 minutes post injection and the brain samples will be dissected and counted as described above.

### Binding Selectivity

Selectivity of [¹⁸F]SRX246 binding will be examined in groups of three mice that will be injected via the tail vein with the blood-brain barrier (BBB)-permeable AVPR1B-selective compound SSR149415 (Nelivaptan, Axon Medchem) (3 mg/Kg) or saline. After 5 minutes, the mice will be injected with 200-400 μCi of [¹⁸F]SRX246. At 30 minutes post injection, the mice will be sacrificed as described above. The selectivity also will be assessed by measuring binding of [¹⁸F]SRX246 (vs. saline-treated controls) in mouse mutants that lack Avpr1a expression (Avpr1a^{tm1Dgen}/J strain Jax strain #005776). In parallel, cold SRX246-based compounds will be synthesized and submitted to the NIMH Psychoactive Drug Screening Program (PDSP) for the evaluation of their pharmacological activity at cloned CNS receptors. Only radiotracers with at least 50-fold selectivity for AVPR1A over other receptors in the screening battery will be pursued.

### Example 5: In Vivo Binding of an AVPR1A Radioligand in Mice

### Radiotracer Synthesis

Once the anticipated binding properties of the SRX246 compound are confirmed, procedures to incorporate radioactive carbon (¹¹C) or fluorine (¹⁸F) into the molecule will be developed. The advantage of ¹⁸F is that it has a much longer half-life (109.8 min) compared to ¹¹C (20.3 min). However, radiolabeling with ¹¹C is often used for organic molecules because of the ease of synthesis and given that these ligands are carbon-based and the incorporation of ¹¹C does not alter their chemical characteristics. In this project, the initial studies with the PET ligand will include labeling with both ¹¹C and ¹⁸F.

### PET Imaging Studies

PET imaging will be performed in group-housed wild-type and Avpr1a^{-/-} females and in singly-housed BDNF-Val66Met+ females that were exposed to social stress from 5 weeks, a condition that causes a dramatic increase in Avpr1a expression in the amygdala, but not in other brain regions or the pituitary. Anesthesia will be delivered through an isoflurane pump that will be connected to a single flexible tube attached to a Y-connector, which diverges into two flexible tubes with one cone each, to be placed over the nose of two mice scanned simultaneously. Once each mouse is fitted with a cone, anesthesia will be induced with 2.5% isoflurane vaporized in medical air and maintained throughout the session with 1.5% isoflurane. The mice will then be placed with the brains in the center of the field of view and secured with elastic restraints. 4 mice per group will be injected via the lateral tail vein with the radiotracer, with a target dose of 100-200 μCi, with high specific activity (>1000 μCi/mmol), and the injected volume not exceeding 200 μL. Scans will be obtained for 60 minutes. One half of the excised tissues will be processed for scintillation counting, while the other half will be preserved in AllProtect (Qiagen) for qPCR analyses. RNA will be extracted, reverse-transcribed to cDNA, and subjected to quantitative real time RT-PCR with Avpr1a primer pairs in the Roche LightCycler480 as described (Ring and Zeltser, J Clin Invest, 120(8), 2931-41 (2010)). Each sample will be assayed in duplicate and mRNA levels normalized to β-actin using the 2^{-ΔΔCT} method.

PET imaging will be performed using the microPET Inveon scanner (Siemens Medical Systems, Knoxville, Tenn.). The Inveon scanner is a rodent-dedicated high resolution PET scanner with radial, tangential and axial resolutions at the center of the field of view of 1.46 mm, 1.49 and 1.15 mm, respectively. It also has a high sensitivity, at 5.8-7.4% and a peak noise equivalent count rate of >1500 kcps for mouse phantoms, indicating that it has a very high degree of accuracy in detecting true counts. Iterative reconstruction will be applied. Registration of reconstructed images, ROI analysis and generation of time activity curves for quantitative analysis will be performed with a combination of MEDx software (Medical Numerics, Germantown Md.) and SPM8. All quantitative analyses will be performed using in-house developed programs scripted with Matlab software (Mathworks, Natick, Mass.). Regions of interest (ROIs) will be delineated on a magnetic resonance imaging (MRI) template from the brain of a sex and age matched mice, set to Paxinos and Watson stereotaxic coordinates. The ROIs will be manually drawn onto the MRI template and will encompass the amygdala, lateral septum, medial prefrontal cortex and hippocampus. Then all PET images will be co-registered and spatially normalized to the combined MRI-ROI template.

The initial outcome measure for the PET scans will be the SUV (standard uptake value), which is the tissue radioactivity concentration normalized to the injected activity and the body weight of the animal. This will be calculated for the time of peak uptake and the area under the curve of the whole scan. A population based input function from mice will be generated, and this will be used to obtain distribution volumes for the regions of interest of interest using a Logan plot-graphic analysis (Logan, et al. J Cereb Blood Flow Metab, 10, 740-7 (1990)) to obtain the equilibrium specific to nonspecific partition coefficient, also referred to as the binding potential relative to non-specific binding (BP_{ND}). PET scans will also be performed using blocking doses (low specific activity) of the cold radiotracer to demonstrate saturability of the binding of the radiotracer to AVPR1A. Avpr1a^{-/-} mutants will be used to establish background levels of binding.

### Example 6: In Vivo Measurement of Increases in Central AVP

Anorexia nervosa patients often exhibit abnormalities in the osmoregulation of plasma AVP, as evidenced by the absence of a tight correlation between plasma sodium and AVP levels during a hypertonic saline challenge (Gold, Kaye, Robertson, & Ebert, 1983 N Engl J Med, 308(19), 1117-1123). Anorexia patients exhibit hypersecretion of AVP into the cerebrospinal fluid (as evidenced by a high ratio of cerebrospinal fluid:plasma AVP), but normal baseline levels of serum AVP and pituitary responsiveness to AVP (Connan, F., Lightman, S. L., Landau, S., Wheeler, M., Treasure, J., & Campbell, I. C. (2007) J Psychiatr Res, 41(1-2), 131-143; Demitrack, M. A., Lesem, M. D., Brandt, H. A., Pigott, T. A., Jimerson, D. C., Altemus, M., & Gold, P. W. (1989) Psychopharmacol Bull, 25(3), 439-443; and Gold, P. W., Kaye, W., Robertson, G. L., & Ebert, M. (1983) N Engl J Med, 308(19), 1117-1123). These observations are consistent with the idea that abnormalities in the AVP system are due to changes in the brain. Thus, dysregulation of the AVP system serves as a biomarker to identify patients that would respond to AVPR1A antagonist treatment.

To develop an assay in live mice that can detect increases in central AVP, it is explored whether altered osmoregulation of plasma AVP correlates with increased risk of anorexic behaviors and/or Avpr1a expression in the amygdala. To this end, the sodium and AVP levels in plasma at baseline and 30 minutes after an intraperitoneal injection of hypertonic saline (600 mOsm/kg, 2% body weight (BW)) versus isotonic saline (290 mOsm/kg, 2% BW) are measured. Next, it is explored whether mice with increased expression of Avp/Avpr1a in the amygdala also have increased ratios of cerebrospinal fluid: plasma AVP.

Increased expression of central AVP indicates that AVP can serve as a biomarker for anorexia nervosa and/or risk for anorexia nervosa, and can be used to identify subject who would benefit from treatment with an AVPR1A antagonist.

### Example 7: Increased Reactivity of the Neuroendocrine AVP System is Associated with Risk of Anorexic Behavior

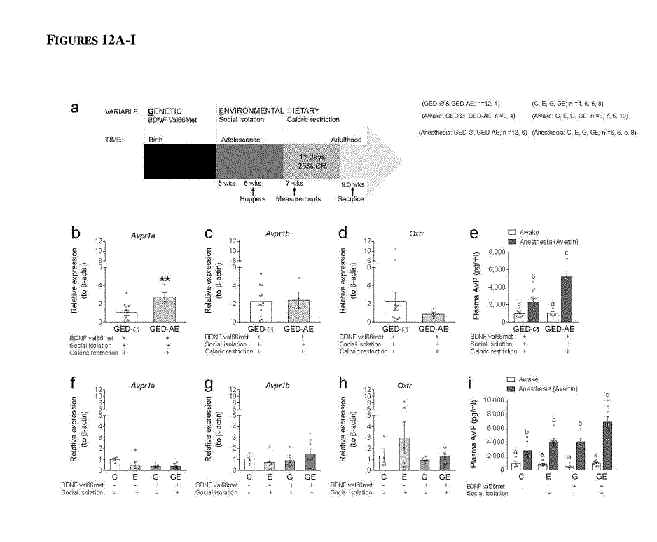

Arginine vasopressin (AVP) is implicated in responsiveness to chronic stress (Lolait, Stewart, Jessop, Young, & O'Carroll, 2007 Endocrinology, 148(2): 849-856) and the AVP system is dysregulated in some AN patients (Gold, Kaye, Robertson, & Ebert, 1983 N Engl J Med, 308(19): 1117-1123). Thus, whether the neuroendocrine AVP system is associated with anorexic behavior in the GED model was explored as set forth in FIG. 12A. To this end, a combination of genetic and pharmacological approaches were used. Using qPCR (LightCycler 480, Roche), expression of Avpr1a, Avpr1b and Oxtr was measured in the anterior pituitary of GED mice that experienced an aphagic episode (GED-AE) vs. mice that did not (GED-Ø). In addition, levels of plasmatic AVP were assessed in the same groups in awake vs. anesthetized animals.

First, 9.5 wk-old GED females that exhibited an aphagic episode (GED-AE) were compared versus age- and exposure-matched littermates that did not (GED-Ø). It was found that a history of aphagic behavior (GED-AE) was associated with a 3-fold increase in Avp in the rostral hypothalamus (which did not reach significance) and a 2.75-fold increase in Avp receptor 1A (Avpr1a) expression in the anterior pituitary (P<0.05, n=10-12) (see FIG. 12B); Avpr1b and Oxytocin receptor (Oxtr) expression were not different (see FIG. 12C (Avpr1b) and FIG. 12D (Oxtr)). It was also observed that the levels of serum AVP in response to avertin anesthesia were increased in GED-AE mice, but were the same in awake mice (FIG. 12E). In addition, baseline levels of plasma AVP during an AE (1.05 pg/ml, n=3) were the same as age-matched GED mice that were eating normally (1.06 pg/ml, n=12).

It was next considered whether changes in the neuroendocrine AVP system might also precede the onset of anorexic behavior. While it was found that exposing hBDNFMet carriers to social isolation stress was associated with increased circulating AVP in response to anesthesia at 7 weeks (GE, stippled bars in FIG. 12I), baseline levels were not altered (GE, unfilled bars in FIG. 12I). Expression of hypothalamic Avp (data not shown) and pituitary Avpr1a, Avpr1b and Oxtr (FIGS. 12F, 12G, and 12H, respectively) were not significantly different between the groups.

It was previously shown that increased plasma AVP is associated with a history (at 9.5 weeks of age, stippled bars in FIG. 12E) and increased risk (at 7 weeks of age, stippled bars in FIG. 12I) of anorexic behavior. It is notable that these measurements were performed on anesthetized mice. In awake mice (unfilled bars in FIGS. 12E and 12I), there was no difference between groups. These findings are similar to reports that serum AVP levels in AN patients are normal at baseline, but are increased in response to a hypertonic saline stress (see Gold, Kaye, Robertson, and Ebert, 1983 N Engl J Med, 308(19): 1117-1123). Thus, elevated AVP in response to a hypertonic saline stress can be used as a diagnostic marker to identify patients who will respond to AVPR1A antagonist treatment.

### Example 8: APR1A is Expressed in an Anorexigenic Subpopulation of Neurons in the Amygdala

Molecular markers for six distinct, but intermingled, neuronal subpopulations in the central nucleus of the amygdala (CeA) that modulate feeding behavior were recently characterized, three of which are anorexigenic (see Kim, J., et al., 2017 Neuron 93(6): 1464-1479 e5, the contents of which are hereby incorporated herein by reference).

The protocol described in Kim et al. was used for single-molecule fluorescence in situ hybridization (smFISH) (ACDBio RNAscope) to map the expression of Avpr1a relative to markers for each neuronal subpopulation—Neurotensin (Nts), Somatostatin (Sst) and Tachykinin 2 (Tac2).

Using two complementary techniques (quantitative PCR for Avpr1a on 200 μm serial sections and single-molecule fluorescence in situ hybridization (smFISH) (ACDBio RNAscope) on 20 μm sections) a peak of Avpr1a expression was identified in the medial division of the central nucleus of the amygdala (CeM) and the extended amygdala (also referred to as the BSTIA) between −1.34 and −1.46 mm from bregma. The number of Avpr1a positive neurons was stable within the peak (between −1.34 and −1.46 mm from bregma), with an average of 50 neurons per hemi-section. In Kim et al. three neuronal subpopulations in the CeM that suppress feeding behavior were characterized. Avpr1a expression was mapped relative to markers for each of the anorexigenic populations—Neurotensin (Nts), Somatostatin (Sst) and Tachykinin 2 (Tac2). It was found that 53.9% of Avpr1a-expressing neurons in the CeM co-express Sst, while 22.4% co-express Nts and 24.7% co-expressTac2, consistent with an anorexigenic function. These findings show that Avpra1 is expressed in an anorexic neuronal subpopulation in the central amygdala, and support the link between Avpr1a and anorexigenic behavior.

### Example 9: Intra-Amygdala AVP Suppresses Food Intake Via AVPR1A

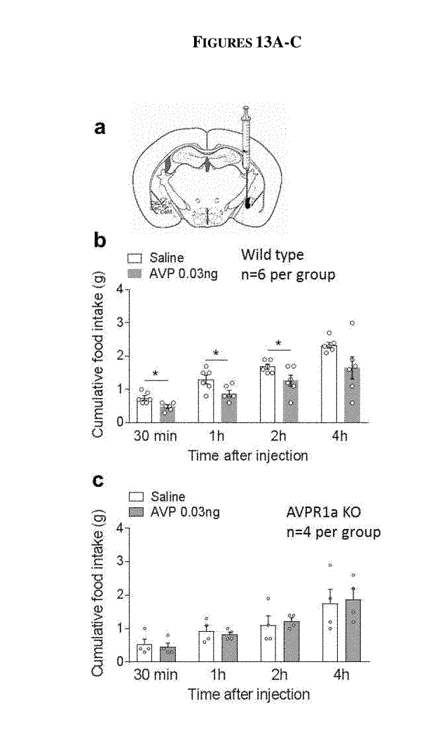

In this example, it was tested whether the anorexic effect of AVP can also be achieved by intra-amygdala injections. The amygdala is a long structure extending from −0.58 mm to −2.5 mm to Bregma. To select the stereotaxic coordinates for the injections, qPCR for Avpr1a on serial 200 μm vibratome sections was performed across the entire amygdala. A peak of expression at −1.4 mm was observed. Unilateral injections of 0.03 ng AVP (vs. saline, in a random crossover design) were made into the amygdala of wild-type mice at this position (dorso-ventral −4.5 mm, lateral −2.4 mm (right side) and rostro-caudal −1.4 mm) (see FIG. 13A).

As shown in FIG. 13B, intra-amygdala AVP reduced food intake in the wild-type mice. Since AVP can also bind to AVPR1b and OxtR, the intra-amygdala injections were repeated in Avpr1a−/− mutants (AVPR1a KO) as described above for the wild-type mice. AVP did not affect food intake in the Avpr1a−/− mutants, supporting the finding that AVPR1A is responsible for food intake (see FIG. 13C).

These studies provide further evidence that AVP signaling via AVPR1A in the amygdala is sufficient to promote anorexigenic behavior.

### Example 10: Peripheral Injection of an AVPR1A Antagonist can Reverse Anorexigenic Behavior Through Actions in the Brain

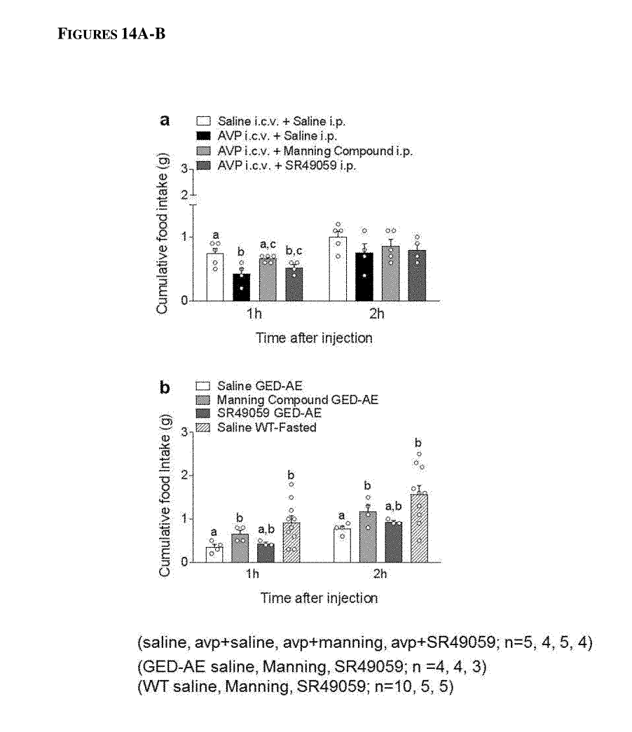

A commercially available AVPR1A antagonist that crosses the blood-brain-barrier (Compound 1) vs. one that does not (SR49059; Sigma) were studied to determine whether critical site(s) of action reside in the brain. First, it was confirmed that the antagonist compounds act as predicted by assessing their ability to block the impact of central injection of AVP (3 ng i.c.v.) on food intake after an overnight fast in wild-type C57BL6/J females. It was found that peripheral injections of Compound 1 (7 μg/kg) can restore 1 hour food intake in wild-type females that received central injections of AVP (3 ng i.c.v.). In contrast, peripheral injection of SR49059 (2 mg/kg) did not significantly reverse AVP-induced hypophagia (FIG. 14A).

Next, the possibility that AVPR1A antagonists could be used to restore feeding behavior in GED mice during an acute aphagic episode was explored. It was studied whether peripheral injection of Compound 1 and SR49059 can reverse anorexigenic behavior in the GED mouse model. To this end, the impact of i.p. injections of 7 g/kg of Compound 1 vs. 2 mg/kg SR49059 vs. saline in the morning of the first day of an aphagic episode (≧18 h of aphagia) was assessed.

Mice were given ad libitum access to food immediately after the injection and food intake was measured after 1 hour and 2 hours. Mice injected with Compound 1 consumed significantly more food than saline-injected GED mice 1 hour after treatment, while injection with the SR49059 compound did not enhance food intake (FIG. 14B).

These studies further support the finding that certain AVPR1A antagonists can be used to treat anorexia.

### REFERENCES

1. Wade, T. D., Bulik, C. M., Neale, M. & Kendler, K. S. Anorexia nervosa and major depression: shared genetic and environmental risk factors. Am J Psychiatry 157, 469-471 (2000).
2. Bulik, C. M., Sullivan, P. F., Fear, J. L. & Joyce, P. R. Eating disorders and antecedent anxiety disorders: a controlled study. Acta Psychiatr Scand 96, 101-107 (1997).
3. Godart, N. T., Flament, M. F., Lecrubier, Y. & Jeammet, P. Anxiety disorders in anorexia nervosa and bulimia nervosa co-morbidity and chronology of appearance. Eur Psychiatry 15, 38-45 (2000).
4. Kaye, W. H., Bulik, C. M., Thornton, L., Barbarich, N. & Masters, K. Comorbidity of anxiety disorders with anorexia and bulimia nervosa. Am J Psychiatry 161, 2215-2221 (2004).
5. Herpertz, S., et al. The diagnosis and treatment of eating disorders. Dtsch Arztebl Int 108, 678-685 (2011).
6. Lebow, J., Sim, L. A., Erwin, P. J. & Murad, M. H. The effect of atypical antipsychotic medications in individuals with anorexia nervosa a systematic review and meta-analysis. Int J Eat Disord 46, 332-339 (2013).
7. Harris, E. C. & Barraclough, B. Excess mortality of mental disorder. Br J Psychiatry 173, 11-53 (1998).
8. Kernie, S. G., Liebl, D. J. & Parada, L. F. BDNF regulates eating behavior and locomotor activity in mice. Embo J 19, 1290-1300 (2000).
9. Egan, M. F., et al. The BDNF val66met polymorphism affects activity-dependent secretion of BDNF and human memory and hippocampal function. Cell 112, 257-269 (2003).
10. Chen, Z. Y., et al. Genetic variant BDNF (Val66Met) polymorphism alters anxiety-related behavior. Science 314, 140-143 (2006).
11. Cao, L., et al. Genetic modulation of BDNF signaling affects the outcome of axonal competition in vivo. Curr Biol 17, 911-921 (2007).
12. Ribases, M., et aL. Association of BDNF with restricting anorexia nervosa and minimum body mass index: a family-based association study of eight European populations. Eur J Hum Genet 13, 428-434 (2005).
13. Akkermann, K., Hiio, K., Villa, I. & Harro, J. Food restriction leads to binge eating dependent upon the effect of the brain-derived neurotrophic factor Val66Met polymorphism. Psychiatry Res 185, 39-43 (2011).
14. Colzato, L. S., Van der Does, A. J., Kouwenhoven, C., Elzinga, B. M. & Hommel, B. BDNF Val66Met polymorphism is associated with higher anticipatory cortisol stress response, anxiety, and alcohol consumption in healthy adults. Psychoneuroendocrinology 36, 1562-1569 (2011).
15. Tocchetto, A., et al. Evidence of association between Val66Met polymorphism at BDNF gene and anxiety disorders in a community sample of children and adolescents. Neurosci Lett 502, 197-200(2011).
16. Aguilera, M., et al. Early adversity and 5-HTT/BDNF genes: new evidence of gene environment interactions on depressive symptoms in a general population. Psychol Med 39, 1425-1432 (2009).
17. Perea, C. S., Paternina, A. C., Gomez, Y. & Lattig, M. C. Negative affectivity moderated by BDNF and stress response. J Affect Disord 136, 767-774 (2012).
18. Raney, T. J., et al. Influence of overanxious disorder of childhood on the expression of anorexia nervosa. Int J Eat Disord 41, 326-332 (2008).
19. Dellava, J. E., et al. Childhood anxiety associated with low BMI in women with anorexia nervosa. Behav Res Ther 48, 60-67 (2010).
20. Workman, J. L., Fonken, L. K, Gusfa, J., Kassouf; KM. & Nelson, R. J. Post-weaning environmental enrichment alters affective responses and interacts with behavioral testing to alter nNOS immunoreactivity. Pharmacol Biochem Behav 100, 25-32 (2011).
21. Niwa, M., et al Adolescent stress-induced epigenetic control of dopaminergic neurons via glucocorticoids. Science 339, 335-339 (2013).
22. Merikangas, K R., et al. Lifetime prevalence of mental disorders in U.S. adolescents: results from the National Comorbidity Survey Replication—Adolescent Supplement (NCS-A). J Am Acad Child Adolesc Psychiatry 49, 980-989 (2010).
23. Spencer, J. L., Waters, E. M., Milner, T. A, Lee, F. S. & McEwen, B. S. BDNF variant Val66Met interacts with estrous cycle in the control of hippocampal function. Proc Natl Acad Sci USA 107, 4395-4400 (2010).
24. Neumark-Sztainer, D., et al. Obesity, disordered eating, and eating disorders in a longitudinal study of adolescents: how do dieters fare 5 years later? J Am Diet Assoc 106, 559-568 (2006).
25. Hudson, J. I., Hiripi, E., Pope, H. G., Jr. & Kessler, R. C. The prevalence and correlates of eating disorders in the National Comorbidity Survey Replication. Biol Psychiatry 61, 348-358 (2007).
26. Hock, H. W. Incidence, prevalence and mortality of anorexia nervosa and other eating disorders. Curr Opin Psychiatry 19, 389-394 (2006).
27. Drewnowski, A., Kurth, C. L. & Krahn, D. D. Weight-loss strategies of young adults: exercise versus dieting. Obes Res 2, 557-561 (1994).
28. Brandenburg, B. M. & Andersen, A. E. Unintentional onset of anorexia nervosa. Eat Weight Disord 12, 97-100 (2007).
29. Bath, K G., et al. Variant Brain Derived Neurotrophic Factor (Valine66Methionine) Polymorphism Contributes to Developmental and Estrous Stage-Specific Expression of Anxiety-Like Behavior in Female Mice. Biol Psychiatry (2012).
30. Knight, Z. A., et al. Molecular profiling of activated neurons by phosphorylated ribosome capture. Cell 151, 1126-1137 (2012).
31. Dardennes, R. M., et al. Family trios analysis of common polymorphisms in the obestatin/ghrelin, BDNF and AGRP genes in patients with Anorexia nervosa: association with subtype, body-mass index, severity and age of onset. Psychoneuroendocrinology 32, 106-113 (2007).
32. Hosang, G. M., Shiles, C., Tansey, K E., McGuffin, P. & Uher, R. Interaction between stress and the BDNF Val66Met polymorphism in depression: a systematic review and metaanalysis. BMC Med 12, 7 (2014).
33. Gentsch, C., Lichtsteiner, M., Frischknecht, K R., Feetr, H. & Siegfried, B. Isolation-induced locomotor hyperactivity and hypoalgesia in rats are prevented by handling and reversed by resocialization. Physiol Behav 43, 13-16 (1988).
34. Rosa, M. L., et al. Routine post-weaning handling of rats prevents isolation rearing-induced deficit in prepulse inhibition. Braz J Med Biol Res 38, 1691-1696 (2005).
35. Sciolino, N. R., et al. Social isolation and chronic handling alter endocannabinoid signaling and behavioral reactivity to context in adult rats. Neuroscience 168, 371-386 (2010).
36. Vinberg, M., et al. The BDNF Val66Met polymorphism: relation to familiar risk of affective disorder, BDNF levels and salivary cortisol. Psychoneuroendocrinology 34, 1380-1389 (2009).
37. Yu, H., et al. Variant brain-derived neurotrophic factor Val66Met polymorphism alters vulnerability to stress and response to antidepressants. J Neurosci 32, 4092-4101 (2012).
38. Gold, P. W., Kaye, W., Robertson, G. L. & Ebert, M. Abnormalities in plasma and cerebrospinal-fluid arginine vasopressin in patients with anorexia nervosa. N Engl J Med 308, 1117-1123 (1983).
39. Meyer, A. H., Langhans, W. & Scharrer, E. Vasopressin reduces food intake in goats. Q J Exp Physiol 74, 465-473 (1989).
40. Langhans, W., Delprete, E. & Scharrer, E. Mechanisms of vasopressin's anorectic effect. Physiol Behav 49, 169-176 (1991).
41. Ikenura, R., Matsuwaki, T., Yamanouchi, K. & Nishihara, M. Involvement of endogenous vasopressin in high plasma osmolality-induced anorexia via V1 receptor-mediated mechanism. The Journal of veterinary medical science/the Japanese Society of Veterinary Science 66, 951-955 (2004).
42. Gesto, M., Soengas, J. L., Rodriguez-Illamola, A. & Miguez, J. M. Arginine vasotocin treatment induces a stress response and exerts a potent anorexigenic effect in rainbow trout, Oncorhynchus mykiss. J Neuroendocrinol 26, 89-99 (2014).
43. Aguilera, G., Subburaju, S., Young, S. & Chen, J. The parvocellular vasopressinergic system and responsiveness of the hypothalamic pituitary adrenal axis during chronic stress. Prog Brain Res 170, 29-39 (2008).
44. Eiland, L. & Romeo, R. D. Stress and the developing adolescent brain. Neuroscience 249, 162-171 (2013).
45. Swartz, J. R, Knodt, A. R., Radtke, S. R. & Hariri, A. R. A neural biomarker of psychological vulnerability to future life stress. Neuron 85, 505-511 (2015).
46. Bachner-Melman, R., et al. Association between a vasopressin receptor AVPR1A promoter region microsatellite and eating behavior measured by a self-report questionnaire (Eating Attitudes Test) in a family-based study of a nonclinical population. Int J Eat Disord 36, 451-460 (2004).
47. Aravich, P. F., Rieg, T. S., Ahmed, I. & Lauterio, T. J. Fluoxetine induces vasopressin and oxytocin abnormalities in food-restricted rats given voluntary exercise: relationship to anorexia nervosa. Brain Res 612, 180-189 (1993).
48. de Magalhaes-Nunes, A. P., et al. Sertraline, a selective serotonin reuptake inhibitor, affects thirst, salt appetite and plasma levels of oxytocin and vasopressin in rats. Exp Physiol 92, 913-922 (2007).
49. Fabio, K., et a. Synthesis and evaluation of potent and selective human V1a receptor antagonists as potential ligands for PET or SPECT imaging. Bioorg Med Chem 20(3), 1337-45 (2012).
50. Zapala, M. A., et al. Adult mouse brain gene expression patterns bear an embryologic imprint. Proc Natl Acad Sci USA 102(29), 10357-62 (2005).
51. Chen, J., et a. Vasopressin does not mediate hypersensitivity of the hypothalamic pituitary adrenal axis during chronic stress. Ann NY Acad Sci 1148, 349-59 (2008).
52. Ring, L. E. and L. M. Zeltser. Disruption of hypothalamic leptin signaling in mice leads to early-onset obesity, but physiological adaptations in mature animals stabilize adiposity levels. J Clin Invest 120(8), 2931-41 (2010).
53. Visser, E. P., et al. Spatial resolution and sensitivity of the Inveon small-animal PET scanner. J Nucl Med 50(1), 139-47 (2009).
54. Goertzen, A. L., et al. NEMA NU 4-2008 comparison of preclinical PET imaging systems. J Nucl Med. 53(8), 1300-9 (2012).
55. Kovacevic, N., et a. A three-dimensional MRI atlas of the mouse brain with estimates of the average and variability. Cereb Cortex 15(5), 639-45 (2005).
56. Logan, J., et al. Graphical analysis of reversible radioligand binding from time-activity measurements applied to [N-11C-methyl]-(−)-cocaine PET studies in human subjects. J Cereb Blood Flow Metab 10, 740-7 (1990).
57. Association AP. Diagnostic and statistical manual of mental disorders, 5th edition (DSM-5). Washington, D.C.: APA; (2013).
58. Hebebrand J, Casper R, Treasure J, Schweiger U. The need to revise the diagnostic criteria for anorexia nervosa. J Neural Transm. 111(7), 827-40 (2004).
59. Health PHSsOiWs, inventorEating Disorders Information Sheet (2000).
60. Walsh B T, Kaplan A S, Attia E, Olmsted M, Parides M, Carter J C, et al. Fluoxetine after weight restoration in anorexia nervosa: a randomized controlled trial. JAMA. 295(22):2605-12 (2006).
61. Connan F, Campbell I C, Katzman M, Lightman S L, Treasure J. A neurodevelopmental model for anorexia nervosa. Physiol Behav. 79(1):13-24 (2003).
62. Jacobi C, Hayward C, de Zwaan M, Kraemer H C, Agras W S. Coming to terms with risk factors for eating disorders: application of risk terminology and suggestions for a general taxonomy. Psychol Bull. 130(1):19-65 (2004).
63. Frank G K, Bailer U F, Henry S E, Drevets W, Meltzer C C, Price J C, et al. Increased dopamine D2/D3 receptor binding after recovery from anorexia nervosa measured by positron emission tomography and [11c]raclopride. Biol Psychiatry. 58(11):908-12 (2005).
64. Kaye W H, Fudge J L, Paulus M. New insights into symptoms and neurocircuit function of anorexia nervosa. Nat Rev Neurosci. 10(8):573-84 (2009).
65. Foerde K, Steinglass J E, Shohamy D, Walsh B T. Neural mechanisms supporting maladaptive food choices in anorexia nervosa. Nat Neurosci. (2015).
66. Casper R C, Sullivan E L, Tecott L. Relevance of animal models to human eating disorders and obesity. Psychopharmacology (Berl). 199(3):313-29 (2008).
67. Kim S F. Animal models of eating disorders. Neuroscience. 211:2-12 (2012).
68. Strober M, Freeman R, Lampert C, Diamond J. The association of anxiety disorders and obsessive compulsive personality disorder with anorexia nervosa: evidence from a family study with discussion of nosological and neurodevelopmental implications. Int J Eat Disord. 40, Suppl:S46-51 (2007).
69. Bulik C M, Sullivan P F, Tozzi F, Furberg H, Lichtenstein P, Pedersen N L. Prevalence, heritability, and prospective risk factors for anorexia nervosa. Arch Gen Psychiatry. 63(3), 305-12 (2006).
70. Pinheiro A P, Bulik C M, Thornton L M, Sullivan P F, Root T L, Bloss C S, et al. Association study of 182 candidate genes in anorexia nervosa. Am J Med Genet B Neuropsychiatr Genet. 153B(5), 1070-80 (2010).
71. Root T L, Szatkiewicz J P, Jonassaint C R, Thornton L M, Pinheiro A P, Strober M, et al. Association of candidate genes with phenotypic traits relevant to anorexia nervosa. Eur Eat Disord Rev. 19(6), 487-93 (2011).
72. Montag C, Basten U, Stelzel C, Fiebach C J, Reuter M. The BDNF Val66Met polymorphism and anxiety: support for animal knock-in studies from a genetic association study in humans. Psychiatry Res. 179(1), 86-90 (2010).
73. Ribases M, Gratacos M, Armengol L, de Cid R, Badia A, Jimenez L, et al. Met66 in the brain-derived neurotrophic factor (BDNF) precursor is associated with anorexia nervosa restrictive type. Mol Psychiatry. 8(8), 745-51 (2003).
74. Gratacos M, Gonzalez J R, Mercader J M, de Cid R, Urretavizcaya M, Estivill X. Brain-derived neurotrophic factor Val66Met and psychiatric disorders: meta-analysis of case-control studies confirm association to substance-related disorders, eating disorders, and schizophrenia. Biol Psychiatry. 61(7), 911-22 (2007).
75. Gatt J M, Nemeroff C B, Dobson-Stone C, Paul R H, Bryant R A, Schofield P R, et al. Interactions between BDNF Val66Met polymorphism and early life stress predict brain and arousal pathways to syndromal depression and anxiety. Mol Psychiatry. 14(7), 681-95 (2009).
76. Lavebratt C, Aberg E, Sjoholm L K, Forsell Y. Variations in FKBP5 and BDNF genes are suggestively associated with depression in a Swedish population-based cohort. J Affect Disord. 125(1-3):249-55 (2010).
77. Schwartz G J, Zeltser L M. Functional organization of neuronal and humoral signals regulating feeding behavior. Annu Rev Nutr. 33, 1-21 (2013).
78. Heinrichs S C, Menzaghi F, Pich E M, Hauger R L, Koob G F. Corticotropin-releasing factor in the paraventricular nucleus modulates feeding induced by neuropeptide Y. Brain Res. 611(1), 18-24 (1993).
79. Menzaghi F, Heinrichs S C, Pich E M, Tilders F J, Koob G F. Functional impairment of hypothalamic corticotropin-releasing factor neurons with immunotargeted toxins enhances food intake induced by neuropeptide Y. Brain Res. 618(1), 76-82 (1993).
80. Smagin G N, Howell L A, Redmann S, Jr., Ryan D H, Harris R B. Prevention of stress-induced weight loss by third ventricle CRF receptor antagonist. Am J Physiol. 276(5 Pt 2), R1461-8 (1999).
81. Ong K K, Northstone K, Wells J C K. Earlier mother's age at menarche predicts rapid infancy growth and childhood obesity. Plos Medicine. 4(4), 737-42 (2007).
82. Luquet S, Perez F A, Hnasko T S, Palmiter R D. NPY/AgRP neurons are essential for feeding in adult mice but can be ablated in neonates. Science. 310(5748), 683-5 (2005).
83. Cai H, Haubensak W, Anthony T E, Anderson D J. Central amygdala PKC-delta neurons mediate the influence of multiple anorexigenic signals. Nat Neurosci. (2014).
84. Lim B K, Huang K W, Grueter B A, Rothwell P E, Malenka R C. Anhedonia requires MC4R-mediated synaptic adaptations in nucleus accumbens. Nature. 487(7406), 183-9 (2012).
85. Calvez J, Fromentin G, Nadkarni N, Darcel N, Even P, Tome D, et al. Inhibition of food intake induced by acute stress in rats is due to satiation effects. Physiol Behav. 104(5), 675-83 (2011).
86. Routtenberg A, Kuznesof A W. Self-starvation of rats living in activity wheels on a restricted feeding schedule. J Comp Physiol Psychol. 64(3), 414-21 (1967).
87. Lantzouni E, Frank G R, Golden N H, Shenker R I. Reversibility of growth stunting in early onset anorexia nervosa: a prospective study. J Adolesc Health. 31(2), 162-5.
88. Walsh B T. The enigmatic persistence of anorexia nervosa. Am J Psychiatry. 170(5), 477-84 (2013).
89. Hellemans K G, Benge L C, Olmstead M C. Adolescent enrichment partially reverses the social isolation syndrome. Brain Res Dev Brain Res. 150(2), 103-15 (2004).
90. Leussis M P, Lawson K, Stone K, Andersen S L. The enduring effects of an adolescent social stressor on synaptic density, part II: Poststress reversal of synaptic loss in the cortex by adinazolam and MK-801. Synapse. 62(3), 185-92 (2008).
91. Aoyagi, T., et al, Enhanced effect of neuropeptide Yon food intake caused by blockade of the V(1A) vasopressin receptor. Eur J Pharmacol, 2009. 622(1-3): p. 32-6.
92. Meyer-Lindenberg, A., et al, Genetic variants in AVPR1A linked to autism predict amygdala activation and personality traits in healthy humans. Mol Psychiatry, 2009. 14(10): p. 968-75.
93. Kruszynski, M., et al, [1-beta-Mercapto-beta,beta-cyclopentamethylenepropionic acid),2-(O-methyl)tyrosine]argine-vasopressin and [1-beta-mercapto-beta,beta-cyclopentamethylenepropionic acid)]argine-vasopressine, two highly potent antagonists of the vasopressor response to arginine-vasopressin. J Med Chem., 1980. 23(4): p. 3564-8.
94. Manning M., et al, Oxytocin and vasopressin agonists and antagonists as research tools and potential therapeutics. J Neuroendocrinol, 2012. 24(4): p. 609-28.
95. Serradeil-Le Gal, C., et al, Characterization of (2S, 4R)-1-[5-chloro-1-[(2,4-dimethoxyphenyl)sulfonyl]-3-(2-methoxy-phenyl)-2-oxo-2,3-dihydro-1H-indol-3-yl]-4-hydroxy-N,N-dimethyl-2-pyrrolidine carboxamide (SSR149415), a selective and orally active vasopressin V1b receptor antagonist. J Pharmacol Exp Ther, 2002. 300(3): p. 1122-30.
96. Guillon, G., et al, The discovery of novel vasopressin V1b receptor ligands for pharmacological, functional and structural investigations. J Neuroendocrinol, 2004. 16(4): p. 356-61.
97. Manaenko, A., et al, Post-treatment with SR49059 improves outcomes following an intracerebral hemorrhagic stroke in mice. Acta Neurochir Suppl, 2011. 111: p. 191-6.
98. Connan, F., Lightman, S. L., Landau, S., Wheeler, M., Treasure, J., & Campbell, I. C. (2007). An investigation of hypothalamic-pituitary-adrenal axis hyperactivity in anorexia nervosa: the role of CRH and AVP. J Psychiatr Res, 41(1-2), 131-143.
99. Demitrack, M. A., Lesem, M. D., Brandt, H. A., Pigott, T. A., Jimerson, D. C., Altemus, M., & Gold, P. W. (1989). Neurohypophyseal dysfunction: implications for the pathophysiokogy of eating disorders. Psychopharmacol Bull, 25(3), 439-443.
100. Gold, P. W., Kaye, W., Robertson, G. L., & Ebert, M. (1983). Abnormalities in plasma and cerebrospinal-fluid arginine vasopressin in patients with anorexia nervosa. N Engl J Med, 308(19), 1117-1123.
101. Lolait, Stewart, Jessop, Young, & O'Carroll, 2007 Endocrinology, 148(2): 849-856.
102. Kim, J., et al., 2017 Neuron 93(6): 1464-1479 e5.

1. A method of treating or preventing an eating disorder in a subject comprising the step of administering a vasopressin V₁ₐ receptor antagonist to the subject, to thereby treat or prevent the eating disorder in the subject.

2. A method of modulating eating behavior in a subject comprising the step of administering a vasopressin V₁ₐ receptor antagonist to the subject, to thereby modulate eating behavior the in the subject.

3. The method of claim 2, wherein eating by the subject is increased.

4. The method of claim 2, wherein caloric intake by the subject is increased.

5. The method of claim 2, wherein caloric restriction by the subject is decreased.

6. The method of claim 1 or 2, wherein the subject is a BDNF Val66Met variant carrier.

7. The method of claim 1 or 2, wherein said treating or preventing comprises increasing the feeding behavior of the subject.

8. The method of claim 1 or 2, wherein the subject has experienced intentional or unintentional weight loss.

9. The method of claim 8, wherein the intentional or the unintentional weight loss is due to a calorie-restricting diet.

10. The method of claim 1, wherein the eating disorder is anorexia nervosa.

11. The method of claim 1 or 2, wherein the subject is a mammal.

12. The method of claim 11, wherein the subject is a human.

13. The method of claim 1 or 2, wherein the subject is female.

14. The method of claim 1 or 2, wherein the subject has, or is at risk of having, anxiety or an anxiety disorder.

15. The method of claim 1 or 2, wherein the subject is an adolescent.

16. The method of claim 1 or 2, wherein the subject is an adult that experienced social stressors during adolescence.

17. The method of claim 1 or 2, wherein the vasopressin V₁ₐ receptor antagonist is a small molecule antagonist.

18. The method of claim 1 or 2, wherein the vasopressin V₁ₐ receptor antagonist is selected from the group consisting of a small interfering RNA (siRNA), a microRNA, an antisense nucleic acid, a ribozyme, an antibody, and a peptide.

19. The method of claim 1 or 2, wherein the vasopressin V₁ₐ receptor antagonist crosses the blood-brain barrier (BBB).

20. The method of claim 1 or 2, wherein the vasopressin V₁ₐ receptor antagonist is selected from the group consisting of: SR49049 (Relcovaptan), atosiban (Tractocile®), conivaptan (YM-087), VPA-985, CL-385004, Vasotocin, SRX251 and SRX246 (Azevan), YM-218 (Astellas), OPC-2158 (Otsuka), and OPC21268, or a pharmaceutically acceptable salt thereof.

21. The method of claim 1 or 2, wherein the vasopressin V₁ₐ receptor antagonist is Compound 1: 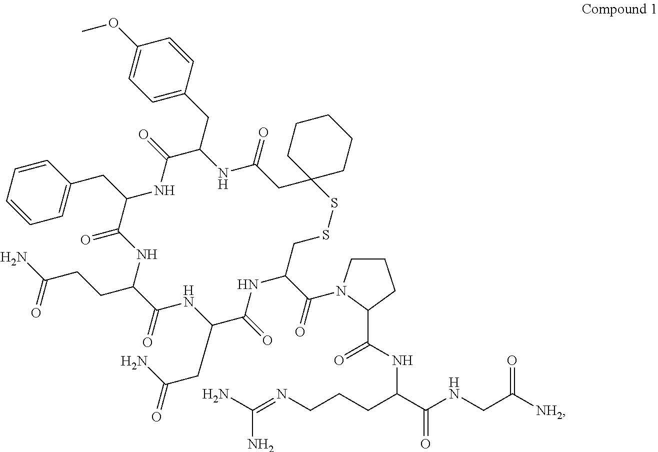 [SMILES: COc1ccc(CC2NC(=O)CC3(CCCCC3)SSCC(C(=O)N3CCCC3C(=O)NC(CCCN=C(N)N)C(=O)NCC(N)=O)NC(=O)C(CC(N)=O)NC(=O)C(CCC(N)=O)NC(=O)C(Cc3ccccc3)NC2=O)cc1]
or a pharmaceutically acceptable salt thereof.

22. The method of claim 1 or 2, wherein the vasopressin V₁ₐ receptor antagonist does not cross the blood brain barrier.

23. The method of claim 1 or 2, wherein the vasopressin V₁ₐ receptor antagonist is administered via injection.

24. The method of claim 1 or 2, wherein the vasopressin V₁ₐ receptor antagonist is administered at in dose of about 1 μg/kg to about 50 μg/kg.

25. The method of claim 24, wherein the vasopressin V₁ₐ receptor antagonist is administered at a dose of about 7 μg/kg.

26. The method of claim 24, wherein the vasopressin V₁ₐ receptor antagonist is administered at a dose of about 40 μg/kg.

27. The method of claim 1 or 2, wherein the vasopressin V₁ₐ receptor antagonist is formulated as a pharmaceutical composition and comprises a pharmaceutically acceptable carrier.
